(19) United States
(12) Patent Application Publication
(10) Pub. No.: US 2026/0096996 A1
(43) Pub. Date: Apr. 9, 2026

(54) PHARMACEUTICAL COMPOSITIONS OF TRETINOIN AND METHODS OF PRODUCING SUCH COMPOSITIONS

(71) Applicant: Douglas Pharmaceuticals Limited, Henderson, Auckland (NZ)

(72) Inventors: Peter William Surman, Auckland (NZ); Zhen Shi, Auckland (NZ); Shih-liang Hsi, Auckland (NZ)

(21) Appl. No.: 19/120,784

(22) PCT Filed: Oct. 13, 2023
(86) PCT No.: PCT/NZ2023/050107

§ 371 Date: Apr. 13, 2025

(30) Foreign Application Priority Data
Oct. 13, 2022 (AU) 2022903010

(51) Int. Cl. A61K 9/48; A61K 9/00; A61K 31/203

(52) U.S. Cl. CPC A61K 9/4875; A61K 9/0053; A61K 9/4808; A61K 9/4825; A61K 9/4833; A61K 9/4858; A61K 31/203

(57) ABSTRACT

Described is a pharmaceutical composition in capsule form. The capsule comprises capsule fill. The capsule fill comprises a therapeutically effective amount of an active agent selected from the group consisting of tretinoin, pharmaceutically acceptable salts thereof, and combinations thereof, at least one carrier, optionally at least one viscosity modifier, optionally at least one chelating agent, optionally at least one antioxidant.

### BACKGROUND

Tretinoin (all-trans retinoic acid (ATRA)) is used topically for the treatment of acne and orally for the treatment of acute promyelocytic leukemia (APL).

In human pharmacokinetics studies, orally administered tretinoin was well absorbed into the systemic circulation, with approximately two-thirds of the administered radiolabel recovered in the urine. The terminal elimination half-life of tretinoin following initial dosing is 0.5 to 2 hours in patients with APL. There is evidence that tretinoin induces its own metabolism. Plasma tretinoin concentrations decrease on average to one-third of their day 1 values during 1 week of continuous therapy (see Tretinoin—tretinoin capsule, liquid filled [package insert], Parsippany, NJ: Teva Pharmaceuticals USA, Inc. 2023).

The recommended dosage of Vesanoid™ capsule (oral tretinoin) for the treatment of APL is usually eight (10 mg) capsules per day split into two equal doses, with instructions to take Vesanoid™ during or immediately after a meal and at the same time each day.

No data on the effect of food on the absorption of tretinoin is presently available. However, the absorption of retinoids as a class has been shown to be enhanced when taken together with food (see Tretinoin—tretinoin capsule, liquid filled [package insert], Parsippany, NJ: Teva Pharmaceuticals USA, Inc. 2023 as previously).

More than half of patients treated with oral tretinoin for APL experience nausea and vomiting (see Tretinoin—tretinoin capsule, liquid filled [package insert], Parsippany, NJ: Teva Pharmaceuticals USA, Inc. 2023 as previously). Release of tretinoin in the stomach may contribute to these deleterious adverse events. Furthermore, vomiting can lead to the dose being incompletely absorbed.

CN112618509 A (Chongqing Huapont Pharm Co Ltd) describes a tretinoin soft capsule with a capsule shell comprising RXL gelatin and capsule contents comprising by weight: tretinoin (4.0), BHA (0.03-0.12), sodium EDTA (0.2-0.7), suspension agent (5.2-7.2), and soybean oil (75.0-92.0). CN112618509 teaches that when the suspending agent exceeded the range, the dissolution rate did not meet requirements. With more suspending agent, the dissolution was slower and only reached 69% at 60 minutes. Further, when the suspending agent exceeded the range, the stability decreased. The retinoic acid impurities and total other impurities was higher under stability testing. The conversion to isotretinoin was 0.26% after months and total other impurities was 0.27% after 12 months. See Comparative Example A, Example 9 and Example 11.

It would be desirable to have an oral dosage which can be taken without consideration of food intake.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

All references cited herein are incorporated herein by reference in their entireties.

### SUMMARY

In one aspect there is provided a pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the active agent has a particle size distribution of D[v,0.5] between about 1 and 10 μm.

In one aspect there is provided a pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
at least one carrier;
at least one viscosity modifier;
optionally at least one chelating agent;
optionally at least one antioxidant;
wherein the active agent has a particle size distribution of D[v,0.5] between about 1 and 10 μm,
wherein the at least one viscosity modifier is present in the capsule fill in a range of about 10 to 25% w/w.

In one aspect there is provided a pharmaceutical composition in capsule form comprising a capsule shell filled with a capsule fill, the capsule fill comprising:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the active agent has a particle size distribution of D[v,0.5] between about 1 and 10 μm.

In one aspect there is provided a pharmaceutical capsule fill composition for a pharmaceutical capsule, the capsule fill comprising:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the active agent has a particle size distribution of D[v,0.5] between about 1 and 10 μm.

In one aspect there is provided a pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the capsule comprises 12 mg of tretinoin and when the capsule is administered to a patient in a fasted state the capsule provides at least one pharmacokinetic parameter selected from the group consisting of:
a mean plasma tretinoin Cₘₐₓ baseline corrected of between about 62 and 243 ng/mL; and
a mean plasma tretinoin AUC_{0-∞} baseline corrected of between about 165 and 507 ng·h/mL.

In one aspect there is provided a pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the capsule provides release of at about least 80% of the active in vitro within about 30 minutes, or at least 85%, or at least 90%, or at least 95%.

In one aspect there is provided a pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
about 50 to 90% w/w of at least one carrier, wherein the carrier is an oil;
about 10 to 25% w/w of at least one viscosity modifier;
optionally at least one chelating agent;
optionally at least one antioxidant.

In one aspect there is provided a pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant.

In one aspect there is provided a liquid pharmaceutical composition comprising:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant, wherein the pharmaceutical composition is formulated for oral administration.

In one aspect there is provided a unit dosage form for oral administration, the unit dosage form comprising:
a capsule shell,
a capsule fill,
wherein the capsule fill comprises;
   a therapeutically effective amount of an active agent selected from the group consisting of tretinoin, pharmaceutically acceptable salts thereof, and combinations thereof,
   at least one carrier;
   optionally at least one viscosity modifier;
   optionally at least one chelating agent;
   optionally at least one antioxidant;
wherein the active agent has a particle size distribution of D[v,0.5] between about 1 and 10 μm.

In one aspect there is provided a method of making a capsule fill for a pharmaceutical composition in capsule form, the method comprising the steps:
adding an active agent selected from the group consisting of tretinoin, pharmaceutically acceptable salts thereof, and combinations thereof to at least one carrier,
wherein the active agent has a particle size distribution of D[v,0.5] between about 1 and 10 μm.

In one aspect there is provided a method of making a pharmaceutical composition in capsule form, the method comprising the step of:
encapsulating the capsule fill, as described herein, in a capsule shell.

For the avoidance of doubt the following examples may apply alone or in any combination of two or more thereof to any one or more of the aspects set forth above where the context allows.

In at least one example, the active agent has a particle size distribution of D[v,0.5] between about 1 and 10 μm, or between about 2 and 10 μm, or between about 3 and 10 μm, or between about 3 and 9 μm, or between about 3 and 8 μm, or between about 4 and 8 μm, or between about 5 and 8 μm, or between about 6 and 8 μm, or about 5 μm.

In at least one example, the active agent has a particle size distribution of D[v,0.5] selected from the group consisting of about 1 μm, about 2 μm, about 3 μm, about 4 μm, about 5 μm, about 6 μm, about 7 μm, about 8 μm, about 9 μm, about 10 μm.

In at least one example, the active agent has a particle size distribution of D[v,0.5] selected from the group consisting of about 5 μm, about 6 μm, about 7 μm, about 8 μm.

In at least one example, the active agent has a particle size distribution of D[v,0.1] of not less than about 0.1 μm, or not less than about 0.2 μm, or not less than about 0.3 μm, or not less than about 0.4 μm, or not less than about 0.5 μm, or not less than about 0.6 μm, or not less than about 0.7 μm, or not less than about 0.8 μm, or not less than about 0.9 μm, or not less than about 1 μm, or not less than about 1.1 μm, or not less than about 1.2 μm, or not less than about 1.3 μm, or not less than about 1.4 μm, or not less than about 1.5 μm, or not less than about 1.6 μm, or not less than about 1.7 μm, or not less than about 1.8 μm, or not less than about 1.9 μm, or not less than about 2 μm.

In at least one example, the active agent has a particle size distribution of D[v,0.1] between about 0.1 and 3 μm, or between about 0.2 and 3 μm, or between about 0.3 and 3 μm, or between about 0.4 and 3 μm, or between about 0.5 and 3 μm, or between about 0.6 and 3 μm, or between about 0.7 and 3 μm, or between about 0.8 and 3 μm, or between about 0.9 and 3 μm, or between about 1 and 3 μm.

In at least one example, the active agent has a particle size distribution of D[v,0.1] between about 0.1 and 2.9 μm, or between about 0.2 and 2.9 μm, or between about 0.3 and 2.9 μm, or between about 0.4 and 2.9 μm, or between about 0.5 and 2.9 μm, or between about 0.6 and 2.9 μm, or between about 0.7 and 2.9 μm, or between about 0.8 and 2.9 μm, or between about 0.9 and 2.9 μm, or between about 1 and 2.9 μm.

In at least one example, the active agent has a particle size distribution of D[v,0.9] of not more than about 35 μm, or not more than about 30 μm, or not more than about 29 μm, or not more than about 28 μm, or not more than about 27 μm, or not more than about 26 μm, or not more than about 25 μm, or not more than about 24 μm, or not more than about 23 μm, not more than about 22 μm, or not more than about 21 μm, not more than about 20 μm, or not more than about 19 μm, or not more than about 18 μm.

In at least one example, the active agent has a particle size distribution of D[v,0.9] between about 8 and 35 μm, or between about 9 and 35 μm, or between about 10 and 35 μm, or between about 10 and 30 μm, or between about 10 and 29 μm, or between about 10 and 28 μm, or between about 10 and 27 μm, or between about 10 and 26 μm, or between about 10 and 25 μm, or between about 10 and 24 μm, or between about 10 and 23 μm, or between about 10 and 22 μm, or between about 10 and 21 μm, or between about 10 and 20 μm, or between about 10 and 19 μm, or between about 11 and 19 μm, or between about 11 and 18 μm, or between about 15 and 18 μm, or between about 11 and 13 μm.

In at least one example, the active agent has a particle size distribution of D[v,0.9] selected from the group consisting of about 11 μm, about 12 μm, about 13 μm, about 14 μm, about 15 μm, about 16 μm, about 17 μm, about 18 μm.

In at least one example, the active agent has a particle size distribution defined by: D[v,0.1] not less than about 0.5 μm; D[v,0.5] between about 1 and 10 μm; and D[v,0.9] not more than 35 μm.

In at least one example, the active agent has a particle size distribution defined by: D[v,0.1] not less than about 0.5 μm; D[v,0.5] between about 1 and 10 μm; and D[v,0.9] not more than 30 μm.

In at least one example, the active agent has a particle size distribution defined by: D[v,0.1] not less than about 0.5 μm; D[v,0.5] between about 1 and 10 μm; and D[v,0.9] not more than 25 μm.

In at least one example, the active agent has a particle size distribution defined by: D[v,0.1] not less than about 0.5 μm; D[v,0.5] between about 1 and 10 μm; and D[v,0.9] not more than 20 μm.

In at least one example, the active agent has a particle size distribution defined by: D[v,0.1] not less than about 0.5 μm; D[v,0.5] between about 3 and 10 μm; and D[v,0.9] not more than 35 μm.

In at least one example, the active agent has a particle size distribution defined by: D[v,0.1] between about 0.5 and 3 μm; D[v,0.5] between about 3 and 10 μm; and D[v,0.9] between about 10 and 30 μm.

In at least one example, the active agent has a particle size distribution defined by: D[v,0.1] between about 0.5 and 3 μm; D[v,0.5] between about 3 and 10 μm; and D[v,0.9] between about 10 and 20 μm.

In at least one example, the active agent has a particle size distribution defined by: D[v,0.1] between about 0.5 and 3 μm; D[v,0.5] between about 3 and 10 μm; and D[v,0.9] between about 10 and 20 μm.

In at least one example, the active agent has a particle size distribution defined by: D[v,0.1] between about 1 and 3 μm; D[v,0.5] between about 3 and 10 μm; and D[v,0.9] between about 10 and 20 μm.

In at least one example, the active agent is tretinoin.

In at least one example, the active agent is present in the capsule or unit dosage form in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w, of the capsule or unit dosage form.

In at least one example, the active agent is present in the capsule fill in a range of about 1 to 55% w/w, or about 2 to 50% w/w, or about 2 to 40% w/w, or about 2 to 30% w/w, or about 2 to 20% w/w, or about 2 to 15% w/w, or about 5 to 15% w/w, or about 5 to 10% w/w of the capsule fill.

In at least one example, the amount of active agent in the capsule or unit dosage form is about 2 to 60 mg, or about 2 to 50 mg, or about 2 to 40 mg, or about 2 to 30 mg, or about 2 to 25 mg.

In at least one example, the amount of active agent in the capsule or unit dosage form is selected from the group consisting of about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 36 mg, about 40 mg, about 45 mg, about 48 mg, about 50 mg and about 60 mg.

In at least one example, the amount of active agent in the capsule is about 5 mg.

In at least one example, the amount of active agent in the capsule is about 10 mg.

In at least one example, the amount of active agent in the capsule is about 12 mg.

In at least one example, the amount of active agent in the capsule is about 15 mg.

In at least one example, the amount of active agent in the capsule is about 20 mg.

In at least one example, the active agent is substantially suspended in the carrier.

In at least one example, the carrier is non-aqueous.

In at least one example, the carrier is a liquid or wax. In at least one example, the carrier is a liquid (for example a liquid at 1 atm and 20° C.)

In at least one example, the carrier comprises an oil.

In at least one example, the carrier is selected from the group consisting of a mineral oil, a vegetable oil, a fatty acid, a fatty acid ester, a glyceride, a fatty alcohol, a hydrogenated oil, a phospholipid, and combinations thereof.

In at least one example, the carrier comprises a vegetable oil.

In at least one example, the carrier comprises soybean oil.

In at least one example, the carrier is present in the capsule or unit dosage form in a range of about 15 to 72% w/w, or about 18 to 72% w/w, or about 18 to 68% w/w, or about 20 to 68% w/w, or about 20 to 64% w/w, or about 21 to 64% w/w, or about 21 to 60% w/w, of the capsule or unit dosage form.

In at least one example, the carrier is present in the capsule fill in a range of about 50 to 90% w/w, or about 60 to 90% w/w, or about 60 to 85% w/w, or about 65 to 85% w/w, or about 65 to 80% w/w, or about 70 to 80% w/w, or about 70 to 75% w/w, or about 50 to 74% w/w, or about 60 to 74% w/w, or about 65 to 74% w/w, or about 70 to 74% w/w of the capsule fill.

In at least one example, the viscosity modifier is selected from the group consisting of hydrogenated vegetable oil, wax, glycerol monostearate, glyceryl behenate, magnesium aluminum silicate, propylene glycol alginate, fatty alcohol and combinations thereof.

In at least one example, the viscosity modifier has a melting point of greater than or equal to 20° C.

In at least one example, the viscosity modifier has a melting point between about 20 and 85° C.

In at least one example, the viscosity modifier is a combination of two or more viscosity modifiers that all have a melting point between about 20 and 85° C.

In at least one example, the viscosity modifier comprises a hydrogenated vegetable oil.

In at least one example, the viscosity modifier comprises hydrogenated vegetable oil Type I and hydrogenated vegetable oil Type II.

In at least one example, the viscosity modifier comprises a wax.

In at least one example the wax is a natural wax, petroleum wax and/or synthetic wax.

In at least one example, the wax is selected from yellow wax, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, or mixtures thereof.

In at least one example, the viscosity modifier comprises a hydrogenated vegetable oil and a wax.

In at least one example, the viscosity modifier comprises hydrogenated vegetable oil Type I and hydrogenated vegetable oil Type II and a wax.

In at least one example, the viscosity modifier is present in the capsule or unit dose form in a range of about 0.6 to 24% w/w, or about 0.6 to 20% w/w, or about 1.5 to 20% w/w, or about 3 to 20% w/w, or about 5 to 20% w/w, or about 5 to 16% w/w, of the capsule or unit dosage form.

In at least one example, the viscosity modifier is present in the capsule fill in a range of about 2 to 30% w/w, or about 2 to 25% w/w, or about 5 to 25% w/w, or about 10 to 25% w/w, or about 15 to 25% w/w, or about 15 to 20% w/w, of the capsule fill.

In at least one example, the chelating agent is selected from the group consisting of ethylenediamine tetraacetic acid (EDTA), disodium edetate, malic acid, citric acid or a pharmaceutically acceptable salt or hydrate thereof, and combinations thereof.

In at least one example, the chelating agent comprises disodium edetate.

In at least one example, the chelating agent is present in the capsule or unit dosage form in a range of about 0.003 to 8% w/w, or about 0.003 to 4% w/w, or about 0.003 to 2.4% w/w, or about 0.003 to 1.6% w/w, or about 0.003 to 0.8% w/w, or about 0.01 to 0.8% w/w, or about 0.03 to 0.8% w/w, or about 0.03 to 0.6% w/w, of the capsule or unit dosage form.

In at least one example, the chelating agent is present in the capsule fill in a range of about 0.01 to 10% w/w, or about 0.01 to 5% w/w, or about 0.01 to 3% w/w, or about 0.01 to 2% w/w, or about 0.01 to 1% w/w, or about 0.05 to 1% w/w, or about 0.1 to 1% w/w, or about 0.1 to 0.8% w/w, of the capsule fill.

In at least one example, the antioxidant is selected from the group consisting of butylated hydroxyanisole (BHA), butylated hydroxytoluene (BHT), propyl gallate, tocopherols (e.g., α-tocopherol (vitamin E)), propionic acid, sodium nitrate, sodium nitrite, citric acid (for example citric acid monohydrate) and combinations thereof.

In at least one example, the antioxidant comprises butylated hydroxyanisole (BHA).

In at least one example, the antioxidant comprises vitamin E.

In at least one example, the antioxidant comprises butylated hydroxyanisole (BHA) and vitamin E.

In at least one example, the antioxidant is present in the capsule or unit dosage form in a range of about 0.0003 to 8%, or about 0.003 to 4% w/w, or about 0.003 to 2.5% w/w, of the capsule or unit dosage form.

In at least one example, the antioxidant is present in the capsule fill in a range of about 0.001 to 10%, or about 0.01 to 5% w/w of the capsule fill, or about 0.01 to 3% w/w, of the capsule fill.

In at least one example, the active agent is present in the capsule or unit dosage form in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form; the at least one carrier is present in a range of about 15 to 72% w/w of the capsule or unit dosage form; the at least one viscosity modifier is present in a range of about 0.6 to 20% w/w of the capsule or unit dosage form.

In at least one example, the active agent is present in the capsule or unit dosage form in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form; the at least one carrier is present in a range of about 15 to 72% w/w of the capsule or unit dosage form; the at least one viscosity modifier is present in a range of about 0.6 to 20% w/w of the capsule or unit dosage form; the at least one antioxidant is present in a range of about 0.003 to 8% w/w of the capsule or unit dosage form.

In at least one example, the active agent is present in the capsule or unit dosage form in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form; the at least one carrier is present in a range of about 15 to 72% w/w of the capsule or unit dosage form; the at least one viscosity modifier is present in a range of about 0.6 to 20% w/w of the capsule or unit dosage form; the at least one chelating agent is present in a range of about 0.003 to 8% w/w of the capsule or unit dosage form; the at least one antioxidant is present in a range of about 0.003 to 8% w/w of the capsule or unit dosage form.

In at least one example, the active agent is present in the capsule or unit dosage form in a range of about 0.6 to 24% w/w of the capsule or unit dosage form; the at least one carrier is present in a range of about 15 to 72% w/w of the capsule or unit dosage form; the at least one viscosity modifier is present in a range of about 0.6 to 20% w/w of the capsule or unit dosage form; the at least one chelating agent is present in a range of about 0.003 to 8% w/w of the capsule or unit dosage form; the at least one antioxidant is present in a range of about 0.003 to 8% w/w of the capsule or unit dosage form.

In at least one example, the active agent is present in the capsule fill in a range of about 1 to 55% w/w, or about 2 to 50% w/w, or about 2 to 40% w/w, or about 2 to 30% w/w, or about 2 to 20% w/w, or about 5 to 20% w/w of the capsule fill; the at least one carrier is present in a range of about 50 to 90% w/w of the capsule fill; the at least one viscosity modifier is present in a range of about 2 to 25% w/w of the capsule fill.

In at least one example, the active agent is present in the capsule fill in a range of about 1 to 55% w/w, or about 2 to 50% w/w, or about 2 to 40% w/w, or about 2 to 30% w/w, or about 2 to 20% w/w, or about 5 to 20% w/w of the capsule fill; the at least one carrier is present in a range of about 50 to 90% w/w of the capsule fill; the at least one viscosity modifier is present in a range of about 10 to 25% w/w of the capsule fill.

In at least one example, the active agent is present in the capsule fill in a range of about 1 to 55% w/w, or about 2 to 50% w/w, or about 2 to 40% w/w, or about 2 to 30% w/w, or about 2 to 20% w/w, or about 5 to 20% w/w of the capsule fill; the at least one carrier is present in a range of about 50 to 90% w/w of the capsule fill; the at least one viscosity modifier is present in a range of about 2 to 25% w/w of the capsule fill; the at least one antioxidant is present in a range of about 0.01 to 10% w/w of the capsule fill.

In at least one example, the active agent is present in the capsule fill in a range of about 1 to 55% w/w, or about 2 to 50% w/w, or about 2 to 40% w/w, or about 2 to 30% w/w, or about 2 to 20% w/w, or about 5 to 20% w/w of the capsule fill; the at least one carrier is present in a range of about 50 to 90% w/w of the capsule fill; the at least one viscosity modifier is present in a range of about 10 to 25% w/w of the capsule fill; the at least one antioxidant is present in a range of about 0.01 to 10% w/w of the capsule fill.

In at least one example, the active agent is present in the capsule fill in a range of about 1 to 55% w/w, or about 2 to 50% w/w, or about 2 to 40% w/w, or about 2 to 30% w/w, or about 2 to 20% w/w, or about 5 to 20% w/w of the capsule fill; the at least one carrier is present in a range of about 50 to 90% w/w of the capsule fill; the at least one viscosity modifier is present in a range of about 2 to 25% w/w of the capsule fill; the at least one chelating agent is present in a range of about 0.01 to 10% w/w of the capsule fill; the at least one antioxidant is present in a range of about 0.01 to 10% w/w of the capsule fill.

In at least one example, the active agent is present in the capsule fill in a range of about 1 to 55% w/w, or about 2 to 50% w/w, or about 2 to 40% w/w, or about 2 to 30% w/w, or about 2 to 20% w/w, or about 5 to 20% w/w of the capsule fill; the at least one carrier is present in a range of about 50 to 90% w/w of the capsule fill; the at least one viscosity modifier is present in a range of about 10 to 25% w/w of the capsule fill; the at least one chelating agent is present in a range of about 0.01 to 10% w/w of the capsule fill; the at least one antioxidant is present in a range of about 0.01 to 10% w/w of the capsule fill.

In at least one example, the active agent is present in the capsule fill in a range of about 2 to 30% w/w of the capsule fill; the at least one carrier is present in a range of about 50 to 90% w/w of the capsule fill; the at least one viscosity modifier is present in a range of about 2 to 25% w/w of the capsule fill; the at least one chelating agent is present in a range of about 0.01 to 10% w/w of the capsule fill; the at least one antioxidant is present in a range of about 0.01 to 10% w/w of the capsule fill.

In at least one example, the active agent is present in the capsule fill in a range of about 2 to 30% w/w of the capsule fill; the at least one carrier is present in a range of about 50 to 90% w/w of the capsule fill; the at least one viscosity modifier is present in a range of about 10 to 25% w/w of the capsule fill; the at least one chelating agent is present in a range of about 0.01 to 10% w/w of the capsule fill; the at least one antioxidant is present in a range of about 0.01 to 10% w/w of the capsule fill.

In at least one example, the capsule and/or capsule fill contains less than about 5% w/w, or less than about 2% w/w, or less than about 1% w/w, or less than about 0.5% w/w, or less than about 0.2% w/w isotretinoin based on the weight of the active agent.

In at least one example, the capsule and/or capsule fill contains less than about 5% w/w isotretinoin based on the weight of the active agent after being stored at about 25 to 30° C. in an opaque container for about 3 months, or about 6 months, or about 9 months, or about 12 months, or about 18 months, or about 24 months, or about 36 months.

In at least one example, the capsule and/or capsule fill contains less than about 2% w/w isotretinoin based on the weight of the active agent after being stored at about 25 to 30° C. in an opaque container for about 3 months, or about 6 months, or about 9 months, or about 12 months, or about 18 months, or about 24 months, or about 36 months.

In at least one example, the capsule and/or capsule fill contains less than about 1% w/w isotretinoin based on the weight of the active agent after being stored at about 25 to 30° C. in an opaque container for about 3 months, or about 6 months, or about 9 months, or about 12 months, or about 18 months, or about 24 months, or about 36 months.

In at least one example, the capsule and/or capsule fill contains less than about 0.5% w/w isotretinoin based on the weight of the active agent after being stored at about 25 to 30° C. in an opaque container for about 3 months, or about 6 months, or about 9 months, or about 12 months, or about 18 months, or about 24 months, or about 36 months.

In at least one example, the capsule and/or capsule fill contains less than about 0.2% w/w isotretinoin based on the weight of the active agent after being stored at about 25 to 30° C. in an opaque container for about 3 months, or about 6 months, or about 9 months, or about 12 months, or about 18 months, or about 24 months, or about 36 months.

In at least one example, the capsule and/or capsule fill contains less than about 0.15% w/w isotretinoin based on the weight of the active agent after being stored at about 25 to 30° C. in an opaque container for about 3 months, or about 6 months, or about 9 months, or about 12 months, or about 18 months, or about 24 months, or about 36 months.

In at least one example, the capsule and/or capsule fill contains less than about 0.1% w/w isotretinoin based on the weight of the active agent after being stored at about 25 to 30° C. in an opaque container for about 3 months, or about 6 months, or about 9 months, or about 12 months, or about 18 months, or about 24 months.

In at least one example the capsule is stored at a relative humidity of 60% or 75%, or 60% at 25° C., or 75% at 30° C.

In at least one example, the capsule is a soft gelatin capsule.

In at least one example, the capsule shell comprises gelatin.

In at least one example, the gelatin is present in the capsule in a range of about 20 to 80% w/w of the capsule shell.

In at least one example, the capsule shell comprises a plasticizer.

In at least one example, the plasticizer is a polyalcohol.

In at least one example, the polyalcohol is selected from the group glycerol, propylene glycol, polyethylene glycol, sorbitol (for example non-crystallizing sorbitol solution), sorbitan, mannitol and combinations thereof.

In at least one example, the plasticizer is selected from the group glycerol, non-crystallizing sorbitol solution and combinations thereof.

In at least one example, the plasticizer is present in the capsule shell in a range of about 1% to 50% w/w, or about 1 to 40% w/w, or about 1 to 30% w/w or about 1 to 25% w/w of the capsule shell.

In at least one example, the capsule shell is about 20 to 70% w/w of the capsule. In at least one example, the capsule shell is about 30 to 60% w/w of the capsule.

In at least one example the capsule and/or unit dosage form is a 12 mg active agent capsule and the capsule shell is about 20 to 70% w/w of the capsule.

In at least one example the capsule and/or unit dosage form is a 12 mg active agent capsule and the capsule shell dry weight is about 30 to 50% w/w of the capsule. In at least one example the capsule and/or unit dosage form is a 12 mg active agent capsule and the capsule shell dry weight is about 40 to 45% w/w of the capsule.

In at least one example the capsule and/or unit dosage form is a 5 mg active agent capsule and the capsule shell dry weight is about 40 to 60% w/w of the capsule. In at least one example the capsule and/or unit dosage form is a 5 mg active agent capsule and the capsule shell dry weight is about 50 to 60% w/w of the capsule.

In at least one example, wherein the capsule and/or unit dosage form is a 12 mg active agent capsule or one or more capsules providing a dose equivalent to a 12 mg active agent capsule and when the capsule is administered to a patient in a fed or fasted state, the capsule provides a mean Tₘₐₓ of the active agent selected from the group consisting of at least about 0.5 hours, at least about 1 hour, at least about 1.5 hours, at least about 2 hours, at least about 2.5 hours, between about 0.5 to 6 hours, between about 1 to 5 hours, between about 1 to 3 hours, between about 2 to 3 hours.

In at least one example, wherein the capsule and/or unit dosage form is a 12 mg active agent capsule or one or more capsules providing a dose equivalent to a 12 mg active agent capsule and when the capsule is administered to a patient in a fed state, the capsule provides a mean plasma tretinoin Cₘₐₓ of at least about 150 ng/mL, or at least about 160 ng/mL, or at least about 180 ng/mL, or at least about 185 ng/mL, or at least about 190 ng/mL, or at least about 200 ng/mL, or at least about 210 ng/mL, or at least about 215 ng/mL, or at least about 220 ng/mL.

In at least one example, wherein the capsule and/or unit dosage form is a 12 mg active agent capsule or one or more capsules providing a dose equivalent to a 12 mg active agent capsule and when the capsule is administered to a patient in a fed state, the capsule provides a mean plasma tretinoin Cₘₐₓ between about 160 and 350 ng/mL, or about 180 and 320 ng/mL, or about 180 and 300 ng/mL, or about 180 and 290 ng/mL, or about 185 and 289 ng/mL, or about 190 and 290 ng/mL, or about 190 and 280 ng/mL, or about 190 and 270 ng/mL, or about 200 and 270 ng/mL, or about 200 and 260 ng/mL, or about 200 and 250 ng/mL, or about 200 and 240 ng/mL, or about 210 and 240 ng/mL, or about 220 and 240 ng/mL.

In at least one example, wherein the capsule and/or unit dosage form is a 12 mg active agent capsule or one or more capsules providing a dose equivalent to a 12 mg active agent capsule and when the capsule is administered to a patient in a fed state, the capsule provides a mean plasma tretinoin AUC_{0-∞} baseline corrected of at least about 300 ng·h/mL, or at least about 320 ng·h/mL, or at least about 330 ng·h/mL, or at least about 340 ng·h/mL, or at least about 350 ng·h/mL, or at least about 360 ng·h/mL, or at least about 370 ng·h/mL, or at least about 380 ng·h/mL, or at least about 390 ng·h/mL, or at least about 400 ng·h/mL.

In at least one example, wherein the capsule and/or unit dosage form is a 12 mg active agent capsule or one or more capsules providing a dose equivalent to a 12 mg active agent capsule and when the capsule is administered to a patient in a fed state, the capsule provides a mean plasma tretinoin AUC_{0-∞} baseline corrected of between about 290 and 562 ng·h/mL, or between about 300 and 550 ng·h/mL, or between about 310 and 530 ng·h/mL, or between about 320 and 520 ng·h/mL, or between about 320 and 510 ng·h/mL, or between about 324 and 506 ng·h/mL.

In at least one example, wherein the capsule and/or unit dosage form is a 12 mg active agent capsule or one or more capsules providing a dose equivalent to a 12 mg active agent capsule and when the capsule is administered to a patient in a fed state, the capsule provides at least one pharmacokinetic parameter selected from the group consisting of:
a mean plasma tretinoin Cₘₐₓ baseline corrected of between about 158 and 321 ng/mL; and
a mean plasma tretinoin AUC_{0-∞} baseline corrected of between about 290 and 562 ng·h/mL.

In at least one example, wherein the capsule and/or unit dosage form is a 12 mg active agent capsule or one or more capsules providing a dose equivalent to a 12 mg active agent capsule and when the capsule is administered to a patient in a fed state, the capsule provides at least one pharmacokinetic parameter selected from the group consisting of:
a mean plasma tretinoin Cₘₐₓ baseline corrected of between about 180 and 300 ng/mL; and
a mean plasma tretinoin AUC_{0-∞} baseline corrected of between about 320 and 520 ng·h/mL.

In at least one example, wherein the capsule and/or unit dosage form is a 12 mg active agent capsule or one or more capsules providing a dose equivalent to a 12 mg active agent capsule and when the capsule is administered to a patient in a fed state, the capsule provides at least one pharmacokinetic parameter selected from the group consisting of:
a mean plasma tretinoin Cₘₐₓ baseline corrected of between about 184 and 289 ng/mL; and
a mean plasma tretinoin AUC_{0-∞} baseline corrected of between about 324 and 506 ng·h/mL.

In at least one example, wherein the capsule and/or unit dosage form is a 12 mg active agent capsule, or one or more capsules providing a dose equivalent to a 12 mg active agent capsule, and when the capsule is administered to a patient in a fasted state, the capsule provides a mean plasma tretinoin Cₘₐₓ of at least about 40 ng/mL, or at least about 50 ng/mL, or at least about 60 ng/mL, or at least about 70 ng/mL, or at least about 80 ng/mL, or at least about 90 ng/mL, or at least about 100 ng/mL, or at least about 110 ng/mL, or at least about 120 ng/mL, or at least about 130 ng/mL, or at least about 135 ng/mL, or at least about 140 ng/mL.

In at least one example, wherein the capsule and/or unit dosage form is a 12 mg active agent capsule, or one or more capsules providing a dose equivalent to a 12 mg active agent capsule, and when the capsule is administered to a patient in a fasted state, the capsule provides a mean plasma tretinoin Cₘₐₓ between about 60 and 250 ng/mL, or between about 62 and 244 ng/mL, or between about 70 and 240 ng/mL, or between about 80 and 230 ng/mL, or between about 90 and 220 ng/mL, or between about 100 and 210 ng/mL, or between about 100 and 200 ng/mL, or between about 110 and 190 ng/mL, or between about 115 and 180 ng/mL, or between about 120 and 170 ng/mL, or between about 130 and 160 ng/mL, or between about 140 and 150 ng/mL.

In at least one example, wherein the capsule and/or unit dosage form is a 12 mg active agent capsule or one or more capsules providing a dose equivalent to a 12 mg active agent capsule and when the capsule is administered to a patient in a fasted state, the capsule provides a mean plasma tretinoin AUC_{0-∞} baseline corrected of at least about 100 ng·h/mL, or at least about 120 ng·h/mL, or at least about 140 ng·h/mL, or at least about 160 ng·h/mL, or at least about 180 ng·h/mL, or at least about 200 ng·h/mL, or at least about 220 ng·h/mL, or at least about 240 ng·h/mL, or at least about 258 ng·h/mL, or at least about 260 ng·h/mL, or at least about 280 ng·h/mL, or at least about 300 ng·h/mL, or at least about 310 ng·h/mL, or at least about 315 ng·h/mL, or at least about 320 ng·h/mL.

In at least one example, wherein the capsule and/or unit dosage form is a 12 mg active agent capsule or one or more capsules providing a dose equivalent to a 12 mg active agent capsule and when the capsule is administered to a patient in a fasted state, the capsule provides a mean plasma tretinoin AUC_{0-∞} baseline corrected of between about 100 and 500 ng·h/mL, or between about 150 and 450 ng·h/mL, or between about 200 and 420 ng·h/mL, or between about 240 and 420 ng·h/mL, or between about 258 and 404 ng·h/mL, or between about 260 and 390 ng·h/mL, or between about 280 and 360 ng·h/mL.

In at least one example, wherein the capsule and/or unit dosage form is a 12 mg active agent capsule or one or more capsules providing a dose equivalent to a 12 mg active agent capsule and when the capsule is administered to a patient in a fasted state, the capsule provides at least one pharmacokinetic parameter selected from the group consisting of:
a mean plasma tretinoin Cₘₐₓ baseline corrected of between about 62 and 243 ng/mL; and
a mean plasma tretinoin AUC_{0-∞} baseline corrected of between about 165 and 507 ng·h/mL.

In at least one example, wherein the capsule and/or unit dosage form is a 12 mg active agent capsule or one or more capsules providing a dose equivalent to a 12 mg active agent capsule and when the capsule is administered to a patient in a fasted state, the capsule provides at least one pharmacokinetic parameter selected from the group consisting of:
a mean plasma tretinoin Cₘₐₓ baseline corrected of between about 100 and 200 ng/mL; and
a mean plasma tretinoin AUC_{0-∞} baseline corrected of between about 240 and 420 ng·h/mL.

In at least one example, wherein the capsule and/or unit dosage form is a 12 mg active agent capsule or one or more capsules providing a dose equivalent to a 12 mg active agent capsule and when the capsule is administered to a patient in a fasted state, the capsule provides at least one pharmacokinetic parameter selected from the group consisting of:
a mean plasma tretinoin Cₘₐₓ baseline corrected of between about 115 and 180 ng/mL; and
a mean plasma tretinoin AUC_{0-∞} baseline corrected of between about 258 and 404 ng·h/mL.

In at least one example, wherein the capsule and/or unit dosage form is a 5 mg active agent capsule or one or more capsules providing a dose equivalent to a 5 mg active agent capsule and when the capsule is administered to a patient in a fed state, the capsule provides a mean plasma tretinoin Cₘₐₓ of at least about 50 ng/mL, or at least about 60 ng/mL, or at least about 70 ng/mL, or at least about 77 ng/mL, or at least about 80 ng/mL, or at least about 85 ng/mL, or at least about 90 ng/mL.

In at least one example, wherein the capsule and/or unit dosage form is a 5 mg active agent capsule or one or more capsules providing a dose equivalent to a 5 mg active agent capsule and when the capsule is administered to a patient in a fed state, the capsule provides a mean plasma tretinoin Cₘₐₓ between about 60 and 150 ng/mL, or between about 70 and 140 ng/mL, or between about 75 and 130 ng/mL, or between about 77 and 120 ng/mL, or between about 80 and 110 ng/mL, or between about 90 and 100 ng/mL.

In at least one example, wherein the capsule and/or unit dosage form is a 5 mg active agent capsule or one or more capsules providing a dose equivalent to a 5 mg active agent capsule and when the capsule is administered to a patient in a fed state, the capsule provides a mean plasma tretinoin AUC_{0-∞} baseline corrected of at least about 100 ng·h/mL, or at least about 110 ng·h/mL, at least about 115 ng·h/mL, at least about 120 ng·h/mL, at least about 125 ng·h/mL, at least about 130 ng·h/mL, at least about 135 ng·h/mL, at least about 140 ng·h/mL, at least about 145 ng·h/mL, at least about 150 ng·h/mL, at least about 165 ng·h/mL.

In at least one example, wherein the capsule and/or unit dosage form is a 5 mg active agent capsule or one or more capsules providing a dose equivalent to a 5 mg active agent capsule and when the capsule is administered to a patient in a fed state, the capsule provides a mean plasma tretinoin AUC_{0-∞} baseline corrected of between about 100 and 250 ng·h/mL, or between about 110 and 245 ng·h/mL, or between about 120 and 240 ng·h/mL, or between about 130 and 220 ng·h/mL, or between about 135 and 211 ng·h/mL, or between about 130 and 210 ng·h/mL, or between about 150 and 200 ng·h/mL.

In at least one example, wherein the capsule and/or unit dosage form is a 5 mg active agent capsule or is one or more capsules providing a dose equivalent to a 5 mg active agent capsule and when the capsule is administered to a patient in a fed state, the capsule provides at least one pharmacokinetic parameter selected from the group consisting of:
a mean plasma tretinoin Cₘₐₓ baseline corrected of between about 65 and 135 ng/mL; and
a mean plasma tretinoin AUC_{0-∞} baseline corrected of between about 120 and 235 ng·h/mL.

In at least one example, wherein the capsule and/or unit dosage form is a 5 mg active agent capsule or is one or more capsules providing a dose equivalent to a 5 mg active agent capsule and when the capsule is administered to a patient in a fed state, the capsule provides at least one pharmacokinetic parameter selected from the group consisting of:
a mean plasma tretinoin Cₘₐₓ baseline corrected of between about 70 and 150 ng/mL; and
a mean plasma tretinoin AUC_{0-∞} baseline corrected of between about 130 and 220 ng·h/mL.

In at least one example, wherein the capsule and/or unit dosage form is a 5 mg active agent capsule or is one or more capsules providing a dose equivalent to a 5 mg active agent capsule and when the capsule is administered to a patient in a fed state, the capsule provides at least one pharmacokinetic parameter selected from the group consisting of:
a mean plasma tretinoin Cₘₐₓ baseline corrected of between about 76 and 120 ng/mL; and
a mean plasma tretinoin AUC_{0-∞} baseline corrected of between about 135 and 211 ng·h/mL.

In at least one example, wherein the capsule and/or unit dosage form is a 5 mg active agent capsule or one or more capsules providing a dose equivalent to a 5 mg active agent capsule and when the capsule is administered to a patient in a fasted state, the capsule provides a mean plasma tretinoin Cₘₐₓ of at least about 20 ng/mL, or at least about 30 ng/mL, or at least about 35 ng/mL, or at least about 40 ng/mL, or at least about 45 ng/mL, or at least about 50 ng/mL, or at least about 55 ng/mL.

In at least one example, wherein the capsule and/or unit dosage form is a 5 mg active agent capsule or one or more capsules providing a dose equivalent to a 5 mg active agent capsule and when the capsule is administered to a patient in a fasted state, the capsule provides a mean plasma tretinoin Cₘₐₓ between about 20 and 110 ng/mL, or between about 25 and 102 ng/mL, or between about 26 and 101 ng/mL, or between about 40 and 90 ng/mL, or between about 48 and 75 ng/mL.

In at least one example, wherein the capsule and/or unit dosage form is a 5 mg active agent capsule or one or more capsules providing a dose equivalent to a 5 mg active agent capsule and when the capsule is administered to a patient in a fasted state, the capsule provides a mean plasma tretinoin AUC_{0-∞} baseline corrected of at least about 40 ng·h/mL, at least about 50 ng·h/mL, at least about 60 ng·h/mL, at least about 70 ng·h/mL, at least about 80 ng·h/mL, at least about 90 ng·h/mL, at least about 100 ng·h/mL, at least about 110 ng·h/mL, at least about 120 ng·h/mL, at least about 130 ng·h/mL.

In at least one example, wherein the capsule and/or unit dosage form is a 5 mg active agent capsule or one or more capsules providing a dose equivalent to a 5 mg active agent capsule and when the capsule is administered to a patient in a fasted state, the capsule provides a mean plasma tretinoin AUC_{0-∞} baseline corrected of between about 50 and 250 ng·h/mL, or between about 60 and 220 ng·h/mL, or between about 65 and 215 ng·h/mL, or between about 68 and 212 ng·h/mL, or between about 70 and 200 ng·h/mL, or between about 80 and 180 ng·h/mL, or between about 90 and 180 ng·h/mL, or between about 100 and 170 ng·h/mL, between about 108 and 169 ng·h/mL, or between about 110 and 150 ng·h/mL.

In at least one example, wherein the capsule and/or unit dosage form is a 5 mg active agent capsule or is one or more capsules providing a dose equivalent to a 5 mg active agent capsule and when the capsule is administered to a patient in a fasted state, the capsule provides at least one pharmacokinetic parameter selected from the group consisting of
a mean plasma tretinoin Cₘₐₓ baseline corrected of between about 25 and 102 ng/mL; and
a mean plasma tretinoin AUC_{0-∞} baseline corrected of between about 68 and 212 ng·h/mL.

In at least one example, wherein the capsule and/or unit dosage form is a 5 mg active agent capsule or is one or more capsules providing a dose equivalent to a 5 mg active agent capsule and when the capsule is administered to a patient in a fasted state, the capsule provides at least one pharmacokinetic parameter selected from the group consisting of
a mean plasma tretinoin Cₘₐₓ baseline corrected of between about 40 and 90 ng/mL; and
a mean plasma tretinoin AUC_{0-∞} baseline corrected of between about 90 and 180 ng·h/mL.

In at least one example, wherein the capsule and/or unit dosage form is a 5 mg active agent capsule or is one or more capsules providing a dose equivalent to a 5 mg active agent capsule and when the capsule is administered to a patient in a fasted state, the capsule provides at least one pharmacokinetic parameter selected from the group consisting of
a mean plasma tretinoin Cₘₐₓ baseline corrected of between about 48 and 75 ng/mL; and
a mean plasma tretinoin AUC_{0-∞} baseline corrected of between about 108 and 169 ng·h/mL.

In at least one example, the capsule and/or unit dosage form provides release of at least about 25% of the active in vitro within about 10 minutes, or at least about 30%, or at least about 35%, or at least about 40%.

In at least one example, the capsule and/or unit dosage form provides release of at least about 65% of the active in vitro within about 15 minutes, or at least about 70%, or at least about 75%, or at least about 80%.

In at least one example, the capsule and/or unit dosage form provides release of at least about 75% of the active in vitro within about 20 minutes, or at least about 80%, or at least about 85%, or at least about 90%.

In at least one example, the capsule and/or unit dosage form provides release of at least about 80% of the active in vitro within about 30 minutes, or at least about 85%, or at least about 90%, or at least about 95%.

In at least one example, the capsule and/or unit dosage form provides release of at least about 98% of the active in vitro within about 45 minutes, or at least about 99%.

In at least one example, the capsule and/or unit dosage form provides release of about 30 to 80%, or about 35 to 60%, or about 38 to 60% of the active in vitro at about 10 minutes.

In at least one example, the capsule and/or unit dosage form provides release of about 60 to 100%, or about 65 to 100%, or about 66 to 100%, of the active in vitro at about 15 minutes.

In at least one example, the capsule and/or unit dosage form provides release of about 70 to 100%, or about 75 to 100%, or about 76 to 100%, of the active in vitro at about 20 minutes.

In at least one example, the capsule and/or unit dosage form provides release of about 70 to 100%, or about 75 to 100%, or about 79 to 100%, of the active in vitro at about 30 minutes.

In at least one example, the capsule and/or unit dosage form provides release of about 75 to 100%, or about 80 to 100%, of the active in vitro at about 45 minutes.

In at least one example, the capsule and/or unit dosage form provides release of the active agent when measured in vitro that substantially corresponds to the following dissolution pattern:
(a) between about 30% and 80% of the total active agent is released after about 10 minutes of measurement in the apparatus;
(b) not less than about 60% of the total active agent is released after 20 minutes of measurement in the apparatus; and
(c) not less than 80% of the total active agent is released after about 30 minutes of measurement in the apparatus.

In at least one example, the capsule and/or unit dosage form provides release of the active agent when measured in vitro that substantially corresponds to the following dissolution pattern:
(a) between about 38 and 60% of the total active agent is released after about 10 minutes of measurement in the apparatus;
(b) between about 75 and 100% of the total active agent is released after 20 minutes of measurement in the apparatus; and
(c) not less than 79% of the total active agent is released after about 30 minutes of measurement in the apparatus.

In at least one example, the capsule and/or unit dosage form provides release of the active agent when measured in vitro that substantially corresponds to the following dissolution pattern:
(a) between about 38 and 60% of the total active agent is released after about 10 minutes of measurement in the apparatus;
(b) between about 65 and 100% of the total active agent is released after 15 minutes of measurement in the apparatus;
(c) not less than 75% of the total active agent is released after about 20 minutes of measurement in the apparatus.

In at least one example, wherein the capsule and/or unit dosage form is a 12 mg active agent capsule, the capsule provides release of at least about 25% of the active in vitro within about 10 minutes, or at least about 30%, or at least about 35%, or at least about 40%.

In at least one example, wherein the capsule and/or unit dosage form is a 12 mg active agent capsule, the capsule provides release of at least about 65% of the active in vitro within about 15 minutes, or at least about 70%, or at least about 75%, or at least about 80%.

In at least one example, wherein the capsule and/or unit dosage form is a 12 mg active agent capsule, the capsule provides release of at least about 75% of the active in vitro within about 20 minutes, or at least about 80%, or at least about 85%, or at least about 90%.

In at least one example, wherein the capsule and/or unit dosage form is a 12 mg active agent capsule, the capsule provides release of at least about 80% of the active in vitro within about 30 minutes, or at least about 85%, or at least about 90%, or at least about 95%.

In at least one example, wherein the capsule and/or unit dosage form is a 12 mg active agent capsule, the capsule provides release of at least about 98% of the active in vitro within about 45 minutes, or at least 99%.

In at least one example, wherein the capsule and/or unit dosage form is a 12 mg active agent capsule, the capsule provides release of about 30 to 80%, or about 35 to 60%, or about 38 to 60% of the active in vitro at about 10 minutes.

In at least one example, wherein the capsule and/or unit dosage form is a 12 mg active agent capsule, the capsule provides release of about 60 to 100%, or about 65 to 100%, or about 66 to 100%, of the active in vitro at about 15 minutes.

In at least one example, wherein the capsule and/or unit dosage form is a 12 mg active agent capsule, the capsule provides release of about 70 to 100%, or about 75 to 100%, or about 76 to 100%, of the active in vitro at about 20 minutes.

In at least one example, wherein the capsule and/or unit dosage form is a 12 mg active agent capsule, the capsule provides release of about 70 to 100%, or about 75 to 100%, or about 79 to 100%, of the active in vitro at about 30 minutes.

In at least one example, wherein the capsule and/or unit dosage form is a 12 mg active agent capsule, the capsule provides release of about 75 to 100%, or about 80 to 100%, of the active in vitro at about 45 minutes.

In at least one example, wherein the capsule and/or unit dosage form is a 12 mg active agent capsule, the capsule provides release of the active agent when measured in vitro that substantially corresponds to the following dissolution pattern:
(a) between about 30% and 80% of the total active agent is released after about 10 minutes of measurement in the apparatus;
(b) not less than about 60% of the total active agent is released after 20 minutes of measurement in the apparatus; and
(c) not less than 80% of the total active agent is released after about 30 minutes of measurement in the apparatus.

In at least one example, wherein the capsule and/or unit dosage form is a 12 mg active agent capsule, the capsule provides release of the active agent when measured in vitro that substantially corresponds to the following dissolution pattern:
(a) between about 38 and 60% of the total active agent is released after about 10 minutes of measurement in the apparatus;
(b) between about 75 and 100% of the total active agent is released after 20 minutes of measurement in the apparatus; and
(c) not less than 79% of the total active agent is released after about 30 minutes of measurement in the apparatus.

In at least one example, wherein the capsule and/or unit dosage form is a 12 mg active agent capsule, the capsule provides release of the active agent when measured in vitro that substantially corresponds to the following dissolution pattern:
(a) between about 38 and 60% of the total active agent is released after about 10 minutes of measurement in the apparatus;
(b) between about 65 and 100% of the total active agent is released after 15 minutes of measurement in the
(c) not less than 75% of the total active agent is released after about 20 minutes of measurement in the apparatus.

In at least one example, the capsule and/or unit dosage form provides release of the active agent when measured using USP (US Pharmacopoeia) 43-NF38 apparatus II (paddles) according to chapter <711>, at speed 100 rpm±4 rpm, in 900 mL per vessel of dissolution medium 0.05 M phosphate buffer, pH 7.8, with 0.5% lauryldimethylamine oxide (LDAO), at 37.0° C.±0.5° C., with spiral capsule sinker, that substantially corresponds to about 30 to 80%, or about 35 to 60%, or about 38 to 60% of the active in vitro at about 10 minutes from start of measurement in the apparatus.

In at least one example, the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer, pH 7.8, with 0.5% lauryldimethylamine oxide (LDAO), at 37.0° C.±0.5° C., with spiral capsule sinker, that substantially corresponds to about 60 to 100%, or about 65 to 100%, or about 66 to 100%, of the active at about 15 minutes from start of measurement in the apparatus.

In at least one example, the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer, pH 7.8, with 0.5% lauryldimethylamine oxide (LDAO), at 37.0° C.±0.5° C., with spiral capsule sinker, that substantially corresponds to about 70 to 100%, or about 75 to 100%, or about 76 to 100%, of the total active at about 20 minutes of from start of measurement in the apparatus.

In at least one example, the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer, pH 7.8, with 0.5% lauryldimethylamine oxide (LDAO), at 37.0° C.±0.5° C., with spiral capsule sinker, that substantially corresponds to about 70 to 100%, or about 75 to 100%, or about 79 to 100%, of the total active at about 30 minutes of from start of measurement in the apparatus.

In at least one example, the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer, pH 7.8, with 0.5% lauryldimethylamine oxide (LDAO), at 37.0° C.±0.5° C., with spiral capsule sinker, that substantially corresponds to about 75 to 100%, or about 80 to 100%, of the total active at about 45 minutes of from start of measurement in the apparatus.

In at least one example, the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer, pH 7.8, with 0.5% lauryldimethylamine oxide (LDAO), at 37.0° C.±0.5° C., with spiral capsule sinker, that substantially corresponds to the following dissolution pattern:
(a) between about 30% and 80% of the total active agent is released after about 10 minutes of measurement in the apparatus;
(b) not less than about 60% of the total active agent is released after 20 minutes of measurement in the apparatus; and
(c) not less than 80% of the total active agent is released after about 30 minutes of measurement in the apparatus.

In at least one example, the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer, pH 7.8, with 0.5% lauryldimethylamine oxide (LDAO), at 37.0° C.±0.5° C., with spiral capsule sinker, that substantially corresponds to the following dissolution pattern:
(a) between about 38 and 60% of the total active agent is released after about 10 minutes of measurement in the apparatus;
(b) between about 75 and 100% of the total active agent is released after 20 minutes of measurement in the apparatus; and
(c) not less than 79% of the total active agent is released after about 30 minutes of measurement in the apparatus.

In at least one example, the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer, pH 7.8, with 0.5% lauryldimethylamine oxide (LDAO), at 37.0° C.±0.5° C., with spiral capsule sinker, that substantially corresponds to the following dissolution pattern:
(a) between about 38 and 60% of the total active agent is released after about 10 minutes of measurement in the apparatus;
(b) between about 65 and 100% of the total active agent is released after 15 minutes of measurement in the
(c) not less than 75% of the total active agent is released after about 20 minutes of measurement in the apparatus.

In at least one example, the pharmaceutical composition is formulated for once daily administration or twice-daily administration to a patient.

In at least one example, the pharmaceutical composition is formulated for oral administration.

In at least one example of the method of making a capsule fill, the active agent is reduced in particle size to the particle size distribution after being added to the at least one carrier.

In at least one example of the method of making a capsule fill, the active agent is milled to the particle size distribution after being added to the at least one carrier.

In at least one example of the method of making a capsule fill, the active agent is bead milled to the particle size distribution after being added to the at least one carrier.

In at least one example of the method of making a capsule fill, the active agent is reduced in particle size to the particle size distribution prior to being added to the at least one carrier.

In at least one example of the method of making a capsule fill, the active agent is milled to the particle size distribution prior to being added to the at least one carrier.

In at least one example of the method of making a capsule fill, the active agent is dry milled to the particle size distribution prior to being added to the at least one carrier.

In at least one example of the method of making a capsule fill, the active agent is jet milled to the particle size distribution prior to being added to the at least one carrier.

In at least one example of the method of making a capsule fill, the active agent is added to at least one carrier and at least one viscosity modifier.

In at least one example of the method of making a capsule fill, the at least one carrier and the at least one viscosity modifier are combined prior to the addition of the active agent. In at least one example, the at least one carrier and the at least one viscosity modifier are combined and heated to about 50 to 80° C., or about 60 to 80° C., or about 65 to 75° C.

In at least one example of the method of making a capsule fill, the antioxidant is added to the carrier at substantially the same time or prior to the active agent.

In at least one example, the combined carrier and viscosity modifier are cooled to about room temperature (for example about 15 to 30° C.) prior to addition of the tretinoin.

For the avoidance of doubt, the embodiments and/or examples may apply alone or in any combination of two or more thereof to any one or more of the aspects set forth above where the context allows.

Further aspects of the present invention and further embodiments and/or examples of the aspects described in the preceding paragraphs will become apparent from the following description, given by way of example and with reference to the accompanying drawings.

### BRIEF DESCRIPTION OF THE FIGURES

The invention will be described in conjunction with the following Figures in which like reference numerals designate like elements and wherein:

FIG. 1 is a graph showing the dissolution profile of 12 mg (Example 3, 12 mg capsule) tretinoin capsule.

FIG. 2 is a graph showing the mean tretinoin plasma concentration after single dose of 12 mg tretinoin (Example 3, 12 mg capsule) tretinoin capsule under fed condition compared to a 20 mg dose of commercially available reference tretinoin soft gel capsule (2×10 mg capsule).

FIG. 3 is a graph showing the mean tretinoin plasma concentration after single dose of 12 mg tretinoin (Example 3, 12 mg capsule) tretinoin capsule under fasted condition compared to a 10 mg dose of commercially available reference tretinoin soft gel capsule (10 mg capsule).

FIG. 4 is a graph showing the dissolution profile under alternative conditions of 12 mg tretinoin (Example 3, 12 mg capsule) 2×5 mg tretinoin capsules (Formulation Example 1, two 5 mg capsules) and commercially available Glenmark Pharmaceuticals tretinoin 10 mg capsules.

### DEFINITION OF TERMS

As used herein the term “active pharmaceutical ingredient” (“API”) or “pharmaceutically active agent” or “active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states.

As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the active agent. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and other known to those of ordinary skill in the pharmaceutical sciences. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In at least one example, the subject is a non-human animal such as a farm animal (e.g., a horse, pig, or cow) or a pet (e.g., a dog or cat). In at least one example, the subject is an elderly human. In at least one example, the subject is a human adult. In at least one example, the subject is a human child. In at least one example, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “therapies” and “therapy” can refer to any method(s), composition(s), and/or agent(s) that can be used in the prevention, treatment and/or management of a disease or condition, or one or more symptoms thereof.

The term “fasted” as used herein is defined as follows: the dosing state which is defined following an overnight fast (wherein 0 caloric intake has occurred) of at least 10 hours. Subjects may administer the dosage form with 240 mL of water. No food should be allowed for at least 4 hours post-dose. Water may be allowed as desired except for one hour before and after drug administration.

The term “fed” as used herein is defined as follows: the dosing state which is defined following an overnight fast (wherein 0 caloric intake has occurred) of at least 10 hours, subjects then begin the recommended high fat meal 30 minutes prior to administration of the drug product. Subjects should eat this meal in 30 minutes or less; however, the drug product should be administered 30 minutes after the start of the meal. The drug product may be administered with 240 mL of water. No food should be allowed for at least 4 hours post-dose. Water may be allowed as desired except for one hour before and after drug administration. A high fat (approximately 50 percent of the total caloric content of the meal is derived from fat) and high calorie (approximately 800 to 1000 calories) meal maybe be used as the test meal under the fed condition. This test meal may derive approximately 100-150, 230-270, and 450-500 calories from protein, carbohydrate, and fat respectively. An example test meal would be two eggs fried in butter, two strips of bacon, two slices of toast with 20 g of butter, four ounces of hash brown potatoes and 200 ml of whole milk.

As used herein, reference to testing for dissolution or release in vitro may be tested using USP (US Pharmacopoeia) 43-NF38 apparatus II (paddles) according to chapter <711>, at speed 100 rpm±4 rpm, in 900 mL per vessel of dissolution medium 0.05 M phosphate buffer, pH 7.8, with 0.5% lauryldimethylamine oxide (LDAO), at 37.0° C.±0.5° C., with spiral capsule sinker.

As used herein, the term “about” when used in conjunction with a stated numerical value or range has the meaning reasonably ascribed to it by a person skilled in the art, i.e. denoting somewhat more or somewhat less than the stated value or range, for example it may vary by as much as 10% or even as much as 9%, 8%, 7%, 6%, 5%, 4%, 3%, 2%, 1%, to the stated numerical value or range.

As used herein, except where the context requires otherwise, the term “comprise” and variations of the term, such as “comprising”, “comprises” and “comprised”, are not intended to exclude further additives, components, integers or steps.

Reference to “capsule fill”, “liquid pharmaceutical composition” and “fill formulation(s)” may be interchangeable.

### DETAILED DESCRIPTION

Tretinoin (Formula I) is a retinoid. Tretinoin is also known as all-trans retinoic acid (ATRA).

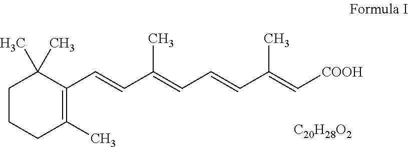

Tretinoin is approved worldwide in topical dosage forms (such as gel, cream and the like) for the treatment of acne vulgaris. Tretinoin is approved in oral dosage form for the treatment of acute promyelocytic leukemia (APL). Described herein is method of treatment of leukemia, for example Acute Promyelocytic Leukemia (“APL”) comprising administration of the pharmaceutical composition, pharmaceutical capsule fill composition and/or unit dosage form as described herein to subject in need thereof.

Oral dosage forms of tretinoin may also have further uses, for example, it has been found to be a potentially effective treatment and/or adjuvant treatment (in combination with immunotherapy) for various solid tumors (see for example WO2019178650A1, “Abstract 3279: All-trans retinoic acid (ATRA) markedly augments anti-tumor immunity, Cancer Res (2019) 79 (13_Supplement): 3279. 1 Jul. 2019 and “Inhibition of myeloid-derived suppressive cell function with all-trans retinoic acid enhanced anti-PD-L1 efficacy in cervical cancer”, Yun Liang1, Wenshan Wang, Xiaojun Zhu, Minghua Yu and Caiyun Zhou; Nature, Scientific Reports, (2022) 12:9619).

The absolute bioavailability of tretinoin is approximately 50%. Following administration of radiolabeled tretinoin 2.75 mg and 50 mg (0.53 to 9.6 times the approved recommended dosage based on 1.7 m), approximately 63% of radioactivity was recovered in the urine within 72 hours and 31% appeared in the feces within 6 days. (see Tretinoin—tretinoin capsule, liquid filled [package insert], Parsippany, NJ: Teva Pharmaceuticals USA, Inc. 2023). However, the oral bioavailability is also unpredictable (see for example, J Natl Cancer Inst. 1993 Jun. 16; 85(12):993-6. doi: 10.1093/jnci/85.12.993. “Variability in the oral bioavailability of all-trans-retinoic acid”, P C Adamson, H C Pitot, F M Balis, J Rubin, R F Murphy, D G Poplack).

The inventors have surprisingly found the oral bioavailability and/or food effect can be further improved by significantly reducing the particle size of the tretinoin.

The disclosure provides a pharmaceutical composition which has higher relative oral bioavailability (for example in the fed state) and/or has an improved food effect to a comparable presently available commercial oral composition. For example, a patient may take the pharmaceutical composition without regard to meals as the bioavailability on fed and fasted dosing are comparable.

The disclosure provides a pharmaceutical composition in capsule form, and/or a capsule fill and/or a unit dosage form comprising tretinoin and/or pharmaceutically acceptable salts thereof, which may have relative bioavailability following oral administration about 3-4-fold higher under patient fasted conditions and/or about 2-fold higher under patient fed conditions when compared with a reference product (marketed 10 mg capsule tretinoin capsules marketed by Glenmark Pharmaceuticals, USA and currently indicated for treatment of Acute Promyelocytic Leukemia (“APL”)). The reference product is also an interchangeable bioequivalent generic product to Vesanoid™ (Roche). The reference product has particle size of D[v,0.1] not less than 2 microns, D[v,0.5]30 to 120 microns, D[v,0.9] not more than 450 microns.

Further or alternatively, the food effect of the composition and/or unit dose as disclosed herein may be estimated (based on the bioavailability studies) to be about 125-160%, compared with around 240-280% for the reference product. These results show higher bioavailability when dosed in the fed state and reduced food effect overall, when compared to the reference product. These were unexpected benefits of the composition/unit dosage as described herein.

Further or alternatively, based on the results of the bioavailability studies, a composition/unit dosage according to the present disclosure with 5 mg tretinoin (5 mg active agent capsule) will be bioequivalent and/or similar to the 10 mg reference product under fed conditions, and/or with less food effect. The 5 mg active agent capsule composition/unit dose according to this disclosure may be bioequivalent to the marketed reference 10 mg active agent product. The benefits may include a smaller physical capsule, which is easier for patients to swallow. The lower dose (for example 5 mg) may allow for more flexibility if doses require titration (for example for new indications).

Described herein is a pharmaceutical composition in capsule form wherein the capsule comprises capsule fill, wherein the capsule fill comprises a therapeutically effective amount of an active agent selected from the group consisting of tretinoin, pharmaceutically acceptable salts thereof, and combinations thereof, at least one carrier, optionally at least one viscosity modifier, optionally at least one chelating agent, optionally at least one antioxidant.

Further or alternatively described herein is a liquid pharmaceutical composition comprising: a therapeutically effective amount of an active agent selected from the group consisting of tretinoin, pharmaceutically acceptable salts thereof, and combinations thereof, at least one carrier, optionally at least one viscosity modifier, optionally at least one chelating agent, optionally at least one antioxidant, wherein the pharmaceutical composition is formulated for oral administration.

Further or alternatively described herein is a pharmaceutical composition in capsule form wherein the capsule comprises capsule fill, wherein the capsule fill comprises: a therapeutically effective amount of an active agent selected from the group consisting of tretinoin, pharmaceutically acceptable salts thereof, and combinations thereof, at least one carrier, optionally at least one viscosity modifier, optionally at least one chelating agent, optionally at least one antioxidant, wherein the active agent has a particle size distribution of D[v,0.5] between about 1 and 10 μm.

Further or alternatively described herein is a pharmaceutical composition in capsule form wherein the capsule comprises capsule fill, wherein the capsule fill comprises: a therapeutically effective amount of an active agent selected from the group consisting of tretinoin, pharmaceutically acceptable salts thereof, and combinations thereof; at least one carrier; at least one viscosity modifier; optionally at least one chelating agent; optionally at least one antioxidant; wherein the active agent has a particle size distribution of D[v,0.5] between about 1 and 10 μm, wherein the at least one viscosity modifier is present in the capsule fill in a range of about 10 to 25% w/w.

Further or alternatively described herein is a pharmaceutical composition in capsule form comprising a capsule shell filled with a capsule fill, the capsule fill comprising: a therapeutically effective amount of an active agent selected from the group consisting of tretinoin, pharmaceutically acceptable salts thereof, and combinations thereof, at least one carrier, optionally at least one viscosity modifier, optionally at least one chelating agent, optionally at least one antioxidant, wherein the active agent has a particle size distribution of D[v,0.5] between about 1 and 10 μm.

Further or alternatively described herein is a pharmaceutical capsule fill composition for a pharmaceutical capsule, the capsule fill comprising: a therapeutically effective amount of an active agent selected from the group consisting of tretinoin, pharmaceutically acceptable salts thereof, and combinations thereof, at least one carrier, optionally at least one viscosity modifier, optionally at least one chelating agent, optionally at least one antioxidant, wherein the active agent has a particle size distribution of D[v,0.5] between about 1 and 10 μm.

Further or alternatively described herein is a pharmaceutical composition in capsule form wherein the capsule comprises capsule fill, wherein the capsule fill comprises: a therapeutically effective amount of an active agent selected from the group consisting of tretinoin, pharmaceutically acceptable salts thereof, and combinations thereof, at least one carrier, optionally at least one viscosity modifier, optionally at least one chelating agent, optionally at least one antioxidant, wherein the capsule comprises 12 mg of tretinoin and when the capsule is administered to a patient in a fasted state the capsule provides at least one pharmacokinetic parameter selected from the group consisting of: a mean plasma tretinoin Cₘₐₓ baseline corrected of between about 62 and 243 ng/mL, and a mean plasma tretinoin AUC_{0-∞} baseline corrected of between about 165 and 507 ng·h/mL.

Further or alternatively described herein is a pharmaceutical composition in capsule form wherein the capsule comprises capsule fill, wherein the capsule fill comprises: a therapeutically effective amount of an active agent selected from the group consisting of tretinoin, pharmaceutically acceptable salts thereof, and combinations thereof, at least one carrier, optionally at least one viscosity modifier, optionally at least one chelating agent, optionally at least one antioxidant, wherein the capsule provides release of at about least 80% of the active in vitro within about 30 minutes, or at least 85%, or at least 90%, or at least 95%.

Further or alternatively described herein is a pharmaceutical composition in capsule form wherein the capsule comprises capsule fill, wherein the capsule fill comprises: a therapeutically effective amount of an active agent selected from the group consisting of tretinoin, pharmaceutically acceptable salts thereof, and combinations thereof; about 50 to 90% w/w of at least one carrier, wherein the carrier is an oil; about 10 to 25% w/w of at least one viscosity modifier; optionally at least one chelating agent; optionally at least one antioxidant.

Further or alternatively described herein is a unit dosage form for oral administration, the unit dosage form comprising: a capsule shell, a capsule fill, wherein the capsule fill comprises a therapeutically effective amount of an active agent selected from the group consisting of tretinoin, pharmaceutically acceptable salts thereof, and combinations thereof, at least one carrier, optionally at least one viscosity modifier, optionally at least one chelating agent, optionally at least one antioxidant, wherein the active agent has a particle size distribution of D[v,0.5] between about 1 and 10 μm.

Further or alternatively described herein is a method of making a capsule fill for a pharmaceutical composition in capsule form, the method comprising the steps: adding an active agent selected from the group consisting of tretinoin, pharmaceutically acceptable salts thereof, and combinations thereof to at least one carrier, wherein the active agent has a particle size distribution of D[v,0.5] between about 1 and 10 μm.

Further or alternatively described herein is a method of making a pharmaceutical composition in capsule form, the method comprising the step of: encapsulating the capsule fill, as described herein, in a capsule shell.

### Active Agent

The active agent is tretinoin (including any hydrates) and/or pharmaceutically acceptable salts thereof, and combinations thereof. The active agent may be tretinoin. When considering the amount, percentage or ratio of the tretinoin in the capsule, capsule fill, unit dosage form or pharmaceutical composition the mass of the water in a hydrate or salt should not be included.

The active agent may be present in the capsule or unit dosage form in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form.

The active agent may be present in the capsule fill in a range of about 1 to 55% w/w, or about 2 to 50% w/w, or about 2 to 40% w/w, or about 2 to 30% w/w, or about 2 to 20% w/w, or about 2 to 15% w/w, or about 5 to 15% w/w, or about 5 to 10% w/w of the capsule fill.

The capsule fill may comprise active agent at a concentration of about 1% w/w, about 2% w/w, about 3% w/w, about 4% w/w, about 5% w/w, about 5.5% w/w, about 6% w/w, about 6.5% w/w, about 7% w/w, about 7.5% w/w, about 8% w/w, about 8.5% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 35% w/w, about 40% w/w, about 45% w/w, or about 50% w/w of the capsule fill.

The active agent may represent about 1 to 30% w/w, or about 1 to 20% w/w, or about 2 to 20% w/w of the total weight of the capsule.

The amount of active agent in the capsule may be about 2 to 60 mg, or about 2 to 50 mg, or about 2 to 40 mg, or about 2 to 30 mg, or about 2 to 25 mg.

The capsule (for example each capsule) may comprise about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 36 mg, about 40 mg, about 45 mg, about 48 mg, about 50 mg or about 60 mg of the active agent.

Reference in this specification to “5 mg capsule”, “5 mg unit dose”, “5 mg active agent capsule”, “tretinoin 5 mg capsule”, “12 mg capsule”, “12 mg unit dose”, “tretinoin 12 mg capsule” or the like, should be taken to be reference to the amount/dose of active agent in the capsule, rather than the whole fill or whole capsule weight.

### Particle Size

There is a wide range of techniques that can be utilized to characterize the particle size of a material. Those skilled in the art also understand that almost all these techniques do not physically measure the actual particle size, as one might measure something with a ruler, but measure a physical phenomenon which is interpreted to indicate a particle size.

For measurements made using a laser diffraction instrument, or an equivalent method known in the art, the term “median particle size” is defined as the median particle diameter as determined on an equivalent spherical particle volume basis. Where the term median is used, it is understood to describe the particle size that divides the population in half such that 50% of the population is greater than or less than this size. The median particle size is often written as D50, D(0.50), D[v,0.5], or D[0.5] or similar. As used herein D50, D[v,0.5], D(0.50) or D[0.5] or similar shall be taken to mean ‘median particle size’.

The term “Dx of the particle size distribution” refers to the xth percentile of the distribution; thus, D90 refers to the 90^{th} percentile, D95 refers to the 95^{th} percentile, and so forth. Taking D90 as an example this can often be written as, D[v,0.9], D(0.90) or D[0.9] or similar. With respect to the median particle size and Dx an upper case D or lowercase d are interchangeable and have the same meaning.

Particle size distribution, for example of an active agent, may be described by the distribution volume at various percentage of the volume, for example at 10%, 50%, and 90% of the volume.

A commonly used way of describing a particle size distribution measured by laser diffraction, or an equivalent method known in the art, is to describe what % of a distribution is under or over a nominated size. The term “percentage less than” also written as “%<” is defined as the percentage, by volume, of a particle size distribution under a nominated size—for example the %<1000 nm. The term “percentage greater than” also written as “%>” is defined as the percentage, by volume, of a particle size distribution over a nominated size—for example the %>1000 nm.

For many of the materials subject to the methods and/or compositions of this disclosure the particle size can be easily measured. Where the active material has poor water solubility and the matrix it is milled in has good water solubility the powder can simply be dispersed in an aqueous solvent. In this scenario the matrix dissolves leaving the active material dispersed in the solvent. This suspension can then be measured by techniques such as photon correlation spectroscopy or laser diffraction.

In the circumstance where an insoluble matrix such as microcrystalline cellulose prevents the measurement of the active material separation techniques such as filtration or centrifugation could be used to separate the insoluble matrix from the active material particles. Other ancillary techniques would also be required to determine if any active material was removed by the separation technique so that this could be taken into account.

In some circumstances image analysis could be used to obtain information about the particle size distribution of the active material. Suitable image measurement techniques might include transmission electron microscopy (TEM), scanning electron microscopy (SEM), optical microscopy and confocal microscopy. In addition to these standard techniques some additional technique would be required to be used in parallel to differentiate the active material and matrix particles. Depending on the chemical makeup of the materials involved possible techniques could be elemental analysis, Raman spectroscopy, FTIR spectroscopy or fluorescence spectroscopy.

In the composition, unit dosage form, capsule and/or method the particles of the active agent (for example tretinoin), may have a particle size (D[v,0.5]), determined on a particle volume basis, that is between about 1 and 10 μm, or about 2 and 10 μm, or between about 3 and 10 μm, or between about 3 and 9 μm, or between about 3 and 8 μm, or between about 4 and 8 μm, or between about 5 and 8 μm, or between about 6 and 8 μm, or about 5 μm, or about 6 μm, or about 7 μm, or about 8 μm.

The particles of the active agent (for example tretinoin) may have a particle size, determined on a particle volume basis, wherein the D[v,0.5] is selected from the group consisting of about 1 μm, about 2 μm, about 3 μm, about 4 μm, about 5 μm, about 6 μm, about 7 μm, about 8 μm, about 9 μm, about 10 μm.

In the composition, unit dosage form, capsule and/or method the particles of the active agent (for example tretinoin), may have a particle size distribution determined on a particle volume basis, wherein the 10^{th} percentile (D[v,0.1]) is not less than about 0.1 μm, or not less than about 0.2 μm, or not less than about 0.3 μm, or not less than about 0.4 μm, or not less than about 0.5 μm, or not less than about 0.6 μm, or not less than about 0.7 μm, or not less than about 0.8 μm, or not less than about 0.9 μm, or not less than about 1 μm, or not less than about 1.1 μm, or not less than about 1.2 μm, or not less than about 1.3 μm, or not less than about 1.4 μm, or not less than about 1.5 μm, or not less than about 1.6 μm, or not less than about 1.7 μm, or not less than about 1.8 μm, or not less than about 1.9 μm, or not less than about 2 μm.

In the composition, unit dosage form, capsule and/or method the particles of the active agent (for example tretinoin), may have a particle size distribution, determined on a particle volume basis, wherein the 10th percentile (D[v,0.1]) is between about 0.1 and 3 μm, or between about 0.2 and 3 μm, or between about 0.3 and 3 μm, or between about 0.4 and 3 μm, or between about 0.5 and 3 μm, or between about 0.6 and 3 μm, or between about 0.7 and 3 μm, or between about 0.8 and 3 μm, or between about 0.9 and 3 μm, or between about 1 and 3 μm. Alternatively, the 10th percentile (D[v,0.1]) is between about 0.1 and 2.9 μm, or between about 0.2 and 2.9 μm, or between about 0.3 and 2.9 μm, or between about 0.4 and 2.9 μm, or between about 0.5 and 2.9 μm, or between about 0.6 and 2.9 μm, or between about 0.7 and 2.9 μm, or between about 0.8 and 2.9 μm, or between about 0.9 and 2.9 μm, or between about 1 and 2.9 μm.

In the composition, unit dosage form, capsule and/or method the particles of the active agent (for example tretinoin) may have a particle size distribution, determined on a particle volume basis, wherein the D[v,0.9] is not more than about 35 μm, or not more than about 30 μm, or not more than about 29 μm, or not more than about 28 μm, or not more than about 27 μm, or not more than about 26 μm, or not more than about 25 μm, or not more than about 24 μm, or not more than about 23 μm, or not more than about 22 μm, or not more than about 21 μm, or not more than about 20 μm, or not more than about 19 μm, or not more than about 18 μm.

The D[v, 0.9] may be between about 8 to 35 μm, between about 9 to 35 μm, or between about 10 to 35 μm, or between about 10 and 30 μm, or between about 10 and 29 μm, or between about 10 and 28 μm, or between about 10 and 27 μm, or between about 10 and 26 μm, or between about 10 to 25 μm, or between about 10 to 24 μm, or between about 10 to 23 μm, or between about 10 to 22 μm, or between about 10 to 21 μm, or between about 10 to 20 μm, or between about 10 and 19 μm, or between about 11 and 19 μm, or between about 11 and 18 μm, or between about 15 and 18 μm, or between about 11 and 13 μm.

The particles of the active agent (for example tretinoin) may have a particle size, determined on a particle volume basis, wherein the D[v,0.9] is selected from the group consisting of about 11 μm, about 12 μm, about 13 μm, about 14 μm, about 15 μm, about 16 μm, about 17 μm, about 18 μm.

In the composition, dosage form, capsule and/or method described herein the active agent (for example tretinoin) may have a particle size distribution defined by:
D[v,0.1] not less than about 0.5 μm, D[v,0.5] between about 1 and 10 μm, and D[v,0.9] not more than 35 μm; or
D[v,0.1] not less than about 0.5 μm, D[v,0.5] between about 1 and 10 μm, and D[v,0.9] not more than 30 μm; or
D[v,0.1] not less than about 0.5 μm, D[v,0.5] between about 1 and 10 μm, and D[v,0.9] not more than 25 μm; or
D[v,0.1] not less than about 0.5 μm, D[v,0.5] between about 1 and 10 μm, and D[v,0.9] not more than 20 μm; or
D[v,0.1] not less than about 0.5 μm, D[v,0.5] between about 3 and 10 μm, and D[v,0.9] not more than 35 μm; or
D[v,0.1] between about 0.5 and 3 μm, D[v,0.5] between about 3 and 10 μm, and D[v,0.9] between about 10 and 30 μm; or
D[v,0.1] between about 0.5 and 3 μm, D[v,0.5] between about 3 and 10 μm, and D[v,0.9] between about 10 and 20 μm; or
D[v,0.1] between about 0.5 and 3 μm, D[v,0.5] between about 3 and 10 μm, and D[v,0.9] between about 10 and 20 μm; or
D[v,0.1] between about 1 and 3 μm, D[v,0.5] between about 3 and 10 μm, and D[v,0.9] between about 10 and 20 μm.

The particle size distribution may have a ratio of D[v,0.1]:D[v,0.5]:D[v,0.9] of about 1:1.5-5:5-20, or about 1:1.5-5:5-15, or about 1:2-4:5-10. Alternatively, the particle size distribution may have a ratio of D[v,0.1]:D[v,0.5]:D[v,0.9] of about 0.2-0.5:1:1.5-4 or about 0.3-0.4:1:2-3. For example, where the ratio is about 1:2-4:5-10 and the D[v,0.1] is about 1-2 μm, the D[v,0.5] may be about 2-8 μm and the D[v,0.9] about 5 to 20 μm.

For comparison, a commercially available tretinoin capsule (10 mg Tretinoin capsules, Glenmark Pharmaceuticals, USA) has particle size of D[v,0.5]30 to 120 μm. The composition, dosage form, capsule and/or method described herein may provide faster dissolution and/or higher bioavailability than the commercially available tretinoin capsule (see for example Examples 4-6).

Reducing the particle size of an active agent can give unpredictable results. In some cases, reducing the particle size can result in particles that can be difficult to handle and/or the dissolution may slow and/or bioavailability be lower due to the particles clumping to effectively form large particles. However, in the present case, reducing the particle size, at least in part resulted in the beneficial improved bioavailability and/or food effect.

The particle size of the active agent may be reduced to the required particle size distribution. In at least one example, the particle size of the active agent may be controlled by particle size reduction through the actions of milling. The milling process may be performed, for example, by bead milling, ball milling or colloid milling. For bead and ball milling these are generally made of ceramic, glass or metal beads. The choice of bead/ball size, bead/ball density, flow rate and residence time inside the milling chamber may determine the particle size distribution. For colloid milling particle size reduction may be achieved through the gap setting between the rotor and stator.

Throughout this specification, unless the context requires otherwise, the phrase “dry mill” or variations, such as “dry milling”, should be understood to refer to milling in at least the substantial absence of liquids. If liquids are present, they are present in such amounts that the contents of the mill retain the characteristics of a dry powder. The phrase “wet mill” or variations, such as “wet milling”, should be understood to refer to milling wherein the particles are dispersed in a liquid.

The active agent may be milled to the particle size distribution after being added to the at least one carrier (for example by wet milling), for example by ball milling. Alternatively, the active agent may be milled to the particle size distribution before being added to the at least one carrier (for example by dry milling), for example by jet milling.

### Pharmacokinetics

In pharmacokinetics the term Cₘₐₓ refers to peak drug concentrations in blood or plasma after dosing. Cₘₐₓ can be influenced by drug dose (higher doses usually produce higher Cₘₐₓ values), how a drug is administered (e.g., higher Cₘₐₓ values may occur after IV bolus dosing compared with oral dosing), and the type of formulation (a higher Cₘₐₓ may occur after dosing with an immediate release oral formulation compared with an extended release formulation). Other drug characteristics such as solubility, permeability, ways in which it is absorbed into the body, metabolism and metabolic products etc., can also influence Cₘₐₓ, which means that although certain projections may be made based on the factors mentioned above, the actual behavior observed is difficult to predict without significant experimentation in humans and may be unexpected.

Compositions and/or unit dosage forms as disclosed herein may provide upon administration to a patient (in a fed or fasted state) a higher mean plasma tretinoin Cₘₐₓ than commercially available oral tretinoin at an equivalent dose. For example, the composition and/or unit does described herein may be more bioavailable than the present commercially available oral tretinoin.

Compositions and/or unit dosage forms as disclosed herein may provide upon administration of a single dose (or capsule) of 12 mg active agent (for example tretinoin) or one or more doses (or capsules) providing a dose equivalent to a 12 mg active agent dose to a patient in a fed state, a mean plasma tretinoin Cₘₐₓ of at least about 150 ng/mL, or at least about 160 ng/mL, or at least about 180 ng/mL, or at least about 190 ng/mL, or at least about 200 ng/mL, or at least about 210 ng/mL, or at least about 220 ng/mL.

Compositions and/or unit dosage forms as disclosed herein may provide upon administration of a single dose (or capsule) of 12 mg active agent (for example tretinoin) or one or more doses (or capsules) providing a dose equivalent to a 12 mg active agent dose to a patient in a fed state, a mean plasma tretinoin Cₘₐₓ of about 160 and 350 ng/mL, or about 180 and 320 ng/mL, or about 180 and 300 ng/mL, or about 180 and 290 ng/mL, or about 190 and 290 ng/mL, or about 190 and 280 ng/mL, or about 190 and 270 ng/mL, or about 200 and 270 ng/mL, or about 200 and 260 ng/mL, or about 200 and 250 ng/mL, or about 200 and 240 ng/mL, or about 210 and 240 ng/mL, or about 220 and 240 ng/mL.

Compositions and/or unit dosage forms as disclosed herein may provide upon administration of a single dose (or capsule) of 5 mg active agent (for example tretinoin) or one or more doses (or capsules) providing a dose equivalent to a 5 mg active agent dose to a patient in a fed state, a mean plasma tretinoin Cₘₐₓ of at least about 50 ng/mL, or at least about 60 ng/mL, or at least about 70 ng/mL, or at least about 77 ng/mL, or at least about 80 ng/mL, or at least about 85 ng/mL, or at least about 90 ng/mL.

Compositions and/or unit dosage forms as disclosed herein may provide upon administration of a single dose (or capsule) of 5 mg active agent (for example tretinoin), or one or more doses (or capsules) providing a dose equivalent to a 5 mg active agent dose to a patient in a fed state, a mean plasma tretinoin Cₘₐₓ of about 60 and 150 ng/mL, or about 70 and 140 ng/mL, or about 75 and 130 ng/mL, or about 77 and 120 ng/mL, or about 80 and 110 ng/mL, or about 90 and 100 ng/mL.

### Tₘₐₓ

Tₘₐₓ refers to the time at which peak drug concentration (Cₘₐₓ) occurs. The Tₘₐₓ may provide an indication to the speed of action of a composition. The compositions and/or unit dosage forms as disclosed herein may provide upon administration of a single dose of 12 mg active agent (for example tretinoin) to patient in a fed and/or fasted state, a mean Tₘₐₓ of the active agent selected from the group consisting of at least about 0.5 hours, or at least about 1 hour, or at least about 1.5 hours, or at least about 2 hours, or at least about 2.5 hours, or between about 0.5 to 6 hours, or between about 1 to 5 hours, or between about 1 to 3 hours, or between about 2 to 3 hours.

### AUC_{0-∞}

The term AUC means Area Under the drug concentration-time Curve in blood or plasma. The AUC reflects the total body exposure to drug after dosing. Again, the size of AUC is influenced by several factors—what dose is administered; ease and speed of drug absorption; how widely the drug is distributed in the body; and rate of drug elimination from the body. All of these variables make it difficult to predict AUC accurately without significant experimentation in humans.

Compositions and/or unit dosage forms as disclosed herein may provide upon administration of a single dose (or capsule) of 12 mg active agent (for example tretinoin) or one or more doses (or capsules) providing a dose equivalent to a 12 mg active agent dose to a patient in a fed state, a mean plasma tretinoin AUC_{0-∞} baseline corrected of at least about 300 ng·h/mL, or at least about 320 ng·h/mL, or at least about 330 ng·h/mL, or at least about 340 ng·h/mL, or at least about 350 ng·h/mL, or at least about 360 ng·h/mL, or at least about 370 ng·h/mL, or at least about 380 ng·h/mL, or at least about 390 ng·h/mL, or at least about 400 ng·h/mL.

Compositions and/or unit dosage forms as disclosed herein may provide upon administration of a single dose (or capsule) of 12 mg active agent (for example tretinoin) or one or more doses (or capsules) providing a dose equivalent to a 12 mg active agent dose to a patient in a fed state, a mean plasma tretinoin AUC_{0-∞} baseline corrected of between about 290 and 562 ng·h/mL, or between about 300 and 550 ng·h/mL, or between about 310 and 530 ng·h/mL, or between about 320 and 520 ng·h/mL, or between about 320 and 510 ng·h/mL, or between about 324 and 506 ng·h/mL.

Compositions and/or unit dosage forms as disclosed herein may provide upon administration of a single dose (or capsule) of 5 mg active agent (for example tretinoin), or one or more doses (or capsules) providing a dose equivalent to a 5 mg active agent dose to a patient in a fed state, a mean plasma tretinoin AUC_{0-∞} baseline corrected of at least about 100 ng·h/mL, or at least about 110 ng·h/mL, or at least about 115 ng·h/mL, or at least about 120 ng·h/mL, or at least about 125 ng·h/mL, or at least about 130 ng·h/mL, or at least about 135 ng·h/mL, or at least about 140 ng·h/mL, or at least about 145 ng·h/mL, or at least about 150 ng·h/mL, or at least about 165 ng·h/mL.

Compositions and/or unit dosage forms as disclosed herein may provide upon administration of a single dose (or capsule) of 5 mg active agent (for example tretinoin) or one or more doses (or capsules) providing a dose equivalent to a 5 mg active agent dose to a patient in a fed state, a mean plasma tretinoin AUC_{0-∞} baseline corrected of between about 100 and 250 ng·h/mL, or between about 110 and 245 ng·h/mL, or between about 120 and 240 ng·h/mL, or between about 130 and 220 ng·h/mL, or between about 135 and 211 ng·h/mL, or between about 130 and 210 ng·h/mL, or between about 150 and 200 ng·h/mL.

The compositions and methods as disclosed herein provide upon administration to a patient in a fed state, for example, a pharmacokinetic parameter selected from the group consisting of:
after administration to a patient in a fed state of a single dose of a tretinoin 12 mg capsule, or one or more doses providing a dose equivalent to a tretinoin 12 mg capsule, a mean plasma tretinoin Cₘₐₓ baseline corrected of between about 150 and 325 ng/mL;
after administration to a patient in a fed state of a single dose of a tretinoin 12 mg capsule, or one or more doses providing a dose equivalent to a tretinoin 12 mg capsule, a mean plasma tretinoin Cₘₐₓ baseline corrected of between about 158 and 321 ng/mL;
after administration to a patient in a fed state of a single dose of a tretinoin 12 mg capsule, or one or more doses providing a dose equivalent to a tretinoin 12 mg capsule, a mean plasma tretinoin Cₘₐₓ baseline corrected selected from, for example, about 150, about 155, about 156, about 157, about 158, about 159, about 159.5, about 160, about 170, about 180, about 190, about 100, about 225, about 150, about 175, about 231, about 300, about 310, about 315, about 320, about 320.9, about 321, about 325, and about 330;
after administration to a patient in a fed state of a single dose of a tretinoin 12 mg capsule, or one or more doses providing a dose equivalent to a tretinoin 12 mg capsule, a mean plasma tretinoin AUC_{0-∞} baseline corrected of between about 290 and 565 ng·h/mL;
after administration to a patient in a fed state of a single dose of a tretinoin 12 mg capsule, or one or more doses providing a dose equivalent to a tretinoin 12 mg capsule, a mean plasma tretinoin AUC_{0-∞} baseline corrected of between about 292 and 562 ng·h/mL;
after administration to a patient in a fed state of a single dose of a tretinoin 12 mg capsule, or one or more doses providing a dose equivalent to a tretinoin 12 mg capsule, a mean plasma tretinoin AUC_{0-∞} baseline corrected selected from, for example, about 280, about 290, about 291, about 291.8 about 295, about 300, about 325, about 350, about 375, about 400, about 405, about 425, about 450, about 475, about 500, about 525, about 550, about 560, about 565, about 570, about 575, and about 580.

When the capsule and/or unit dosage form is a 12 mg active agent capsule or one or more capsules providing a dose equivalent to a 12 mg active agent capsule and when the capsule is administered to a patient in a fed state the capsule may provide at least one pharmacokinetic parameter selected from the group consisting of: a mean plasma tretinoin Cₘₐₓ baseline corrected of between about 158 and 321 ng/mL; and a mean plasma tretinoin AUC_{0-∞} baseline corrected of between about 290 and 562 ng·h/mL.

When the capsule and/or unit dosage form is a 12 mg active agent capsule or one or more capsules providing a dose equivalent to a 12 mg active agent capsule and when the capsule is administered to a patient in a fed state the capsule may provide at least one pharmacokinetic parameter selected from the group consisting of: a mean plasma tretinoin Cₘₐₓ baseline corrected of between about 180 and 300 ng/mL; and a mean plasma tretinoin AUC_{0-∞} baseline corrected of between about 320 and 520 ng·h/mL.

When the capsule and/or unit dosage form is a 12 mg active agent capsule or one or more capsules providing a dose equivalent to a 12 mg active agent capsule and when the capsule is administered to a patient in a fed state the capsule may provide at least one pharmacokinetic parameter selected from the group consisting of: a mean plasma tretinoin Cₘₐₓ baseline corrected of between about 184 and 289 ng/mL; and a mean plasma tretinoin AUC_{0-∞} baseline corrected of between about 324 and 506 ng·h/mL.

The compositions and/or unit dosage forms as disclosed herein may provide upon administration to a patient in a fed state, for example, a pharmacokinetic parameter selected from the group consisting of:
after administration to a patient in a fed state of a single dose of a tretinoin 5 mg capsule, or one or more doses providing a dose equivalent to a tretinoin 5 mg capsule, a mean plasma tretinoin Cₘₐₓ baseline corrected of between about 60 and 140 ng/mL;
after administration to a patient in a fed state of a single dose of a tretinoin 5 mg capsule, or one or more doses providing a dose equivalent to a tretinoin 5 mg capsule, a mean plasma tretinoin Cₘₐₓ baseline corrected of between about 66 and 134 ng/mL;
after administration to a patient in a fed state of a single dose of a tretinoin 5 mg capsule, or one or more doses providing a dose equivalent to a tretinoin 5 mg capsule, a mean plasma tretinoin Cₘₐₓ baseline corrected selected from, for example, about 50, about 60, about 62, about 63, about 64, about 65, about 66, about 67, about 68 about 69, about 70, about 72, about 75, about 80, about 82, about 85, about 90, about 92, about 95, about 96.3 about 97, about 100, about 110, about 115, about 120, about 125, about 130, about 131, about 132, about 133, about 134, about 135, about 136, about 137, about 138, about 139, and about 140 ng/mL;
after administration to a patient in a fed state of a single dose of a tretinoin 5 mg capsule, or one or more doses providing a dose equivalent to a tretinoin 5 mg capsule, a mean plasma tretinoin AUC_{0-∞} baseline corrected of between about 100 and about 250 ng·h/mL;
after administration to a patient in a fed state of a single dose of a tretinoin 5 mg capsule, or one or more doses providing a dose equivalent to a tretinoin 5 mg capsule, a mean plasma tretinoin AUC_{0-∞} baseline corrected of between about 120 and about 235 ng·h/mL;
after administration to a patient in a fed state of a single dose of a tretinoin 5 mg capsule, or one or more doses providing a dose equivalent to a tretinoin 5 mg capsule, a mean plasma tretinoin AUC_{0-∞} baseline corrected selected from, for example, about 115, about 120, about 122, about 125, about 130, about 135, about 140, about 142, about 145, about 150, about 152, about 155, about 160, about 162, about 165, about 168.9, about 170, about 172, about 175, about 180, about 182, about 185, about 190, about 192, about 195, about 200, about 202, about 205, about 210, about 212, about 215, about 220, about 222, about 225, about 230, about 232, about 235, about 240, about 242, about 245, and about 250.

When the capsule and/or unit dosage form is a 5 mg active agent capsule or one or more capsules providing a dose equivalent to a 5 mg active agent capsule and when the capsule is administered to a patient in a fed state the capsule may provide at least one pharmacokinetic parameter selected from the group consisting of: a mean plasma tretinoin Cₘₐₓ baseline corrected of between about 65 and 135 ng/mL; and a mean plasma tretinoin AUC_{0-∞} baseline corrected of between about 120 and 235 ng·h/mL.

When the capsule and/or unit dosage form is a 5 mg active agent capsule or one or more capsules providing a dose equivalent to a 5 mg active agent capsule and when the capsule is administered to a patient in a fed state the capsule may provide at least one pharmacokinetic parameter selected from the group consisting of: a mean plasma tretinoin Cₘₐₓ baseline corrected of between about 70 and 150 ng/mL; and a mean plasma tretinoin AUC_{0-∞} baseline corrected of between about 130 and 220 ng·h/mL.

When the capsule and/or unit dosage form is a 5 mg active agent capsule or one or more capsules providing a dose equivalent to a 5 mg active agent capsule and when the capsule is administered to a patient in a fed state the capsule may provide at least one pharmacokinetic parameter selected from the group consisting of: a mean plasma tretinoin Cₘₐₓ baseline corrected of between about 76 and 120 ng/mL; and a mean plasma tretinoin AUC_{0-∞} baseline corrected of between about 135 and 211 ng·h/mL.

### Food Effect

The term “food effect” refers to a somewhat unpredictable phenomenon that can influence the absorption of drugs from the gastrointestinal tract following oral administration. A food effect can be designated “negative” when absorption is decreased, or “positive” when absorption is increased and manifested as an increase in oral bioavailability (as reflected by total exposure, usually defined as AUC). Alternatively, food effects can refer to changes in maximum concentration (Cₘₐₓ), or the time to reach maximum concentration (Tₘₐₓ), independently of overall absorption. As a result, some drugs have to be taken in either fasted or fed conditions to achieve the optimum effect. For example, patients may be instructed to take a drug with a meal, before a meal (e.g., one hour before a meal), or after a meal (e.g., two hours after a meal), making compliance by the patient more difficult and as a result less likely. However, many drugs are unaffected by food, and thus, can be taken in either a fasted or a fed condition.

The dosage instructions for the treatment of APL for present commercially available oral tretinoin is usually eight (10 mg) capsules per day split into two equal doses, to be taken during or immediately after a meal and at the same time each day. The instructions to take during or after a meal are believed to be due to the food effect of the medication, for example there is lower bioavailability when taken without food.

The composition and/or unit dosage form of the present disclosure has less food effect than prior art formulations when administered to patient, i.e., the inventive formulation is less dependent on the presence of food in the patient to achieve high bioavailability. For example, prior art tretinoin formulations must be taken with food to achieve high bioavailability. The inventors have unexpectedly discovered a tretinoin composition that achieves high bioavailability with a significantly reduced food effect compared to existing tretinoin compositions.

Surprisingly, the compositions and/or unit dosage forms as disclosed herein may provide upon administration to a patient in a fasted state a higher mean plasma tretinoin Cₘₐₓ than commercially available oral tretinoin at an equivalent dose.

Compositions and/or unit dosage forms as disclosed herein may provide upon administration of a single dose (or capsule) of 12 mg active agent (for example tretinoin), or one or more doses (or capsules) providing a dose equivalent to a 12 mg active agent dose, to a patient in a fasted state, a mean plasma tretinoin Cₘₐₓ of at least about 40 ng/mL, or at least about 50 ng/mL, or at least about 60 ng/mL, or at least about 70 ng/mL, or at least about 80 ng/mL, or at least about 90 ng/mL, or at least about 100 ng/mL, or at least about 110 ng/mL, or at least about 120 ng/mL, or at least about 130 ng/mL, or at least about 135 ng/mL, or at least about 140 ng/mL.

Compositions and/or unit dosage forms as disclosed herein may provide upon administration of a single dose (or capsule) of 12 mg active agent (for example tretinoin), or one or more doses (or capsules) providing a dose equivalent to a 12 mg active agent dose, to a patient in a fasted state, a mean plasma tretinoin Cₘₐₓ between about 60 and 250 ng/mL, or about 62 and 243 ng/mL, or about 70 and 240 ng/mL, or about 80 and 230 ng/mL, or about 90 and 220 ng/mL, or about 100 and 210 ng/mL, or about 100 and 200 ng/mL, or about 110 and 190 ng/mL, or about 115 and 180 ng/mL, or about 120 and 170 ng/mL, or about 130 and 160 ng/mL, or about 140 and 150 ng/mL.

Compositions and/or unit dosage forms as disclosed herein may provide upon administration of a single dose (or capsule) of 5 mg active agent (for example tretinoin), or one or more doses (or capsules) providing a dose equivalent to a 5 mg active agent dose, to a patient in a fasted state, a mean plasma tretinoin Cₘₐₓ of at least about 20 ng/mL, or at least about 30 ng/mL, or at least about 35 ng/mL, or at least about 40 ng/mL, or at least about 45 ng/mL, or at least about 50 ng/mL, or at least about 55 ng/mL.

Compositions and/or unit dosage forms as disclosed herein may provide upon administration of a single dose (or capsule) of 5 mg active agent (for example tretinoin), or one or more doses (or capsules) providing a dose equivalent to a 5 mg active agent dose, to a patient in a fasted state, a mean plasma tretinoin Cₘₐₓ between about 20 and 110 ng/mL, or between about 25 and 102 ng/mL, or between about 26 and 101 ng/mL, or between about 40 and 90 ng/mL, or between about 48 and 75 ng/mL.

Compositions and/or unit dosage forms as disclosed herein may provide upon administration of a single dose (or capsule) of 12 mg active agent (for example tretinoin), or one or more doses (or capsules) providing a dose equivalent to a 12 mg active agent dose, to a patient in a fasted state, a mean plasma tretinoin AUC_{0-∞} baseline corrected of at least about 100 ng·h/mL, or at least about 120 ng·h/mL, or at least about 140 ng·h/mL, or at least about 160 ng·h/mL, or at least about 180 ng·h/mL, or at least about 200 ng·h/mL, or at least about 220 ng·h/mL, or at least about 240 ng·h/mL, or at least about 258 ng·h/mL, or at least about 260 ng·h/mL, or at least about 280 ng·h/mL, or at least about 300 ng·h/mL, or at least about 310 ng·h/mL, or at least about 315 ng·h/mL, or at least about 320 ng·h/mL.

Compositions and/or unit dosage forms as disclosed herein may provide upon administration of a single dose (or capsule) of 12 mg active agent (for example tretinoin), or one or more doses (or capsules) providing a dose equivalent to a 12 mg active agent dose, to a patient in a fasted state, a mean plasma tretinoin AUC_{0-∞} baseline corrected of between about 100 and 500 ng·h/mL, or between about 150 and 450 ng·h/mL, or between about 200 and 420 ng·h/mL, or between about 240 and 420 ng·h/mL, or between about 258 and 404 ng·h/mL, or between about 260 and 390 ng·h/mL, or between about 280 and 360 ng·h/mL.

Compositions and/or unit dosage forms as disclosed herein may provide upon administration of a single dose (or capsule) of 5 mg active agent (for example tretinoin), or one or more doses (or capsules) providing a dose equivalent to a 5 mg active agent dose, to a patient in a fasted state, a mean plasma tretinoin AUC_{0-∞} baseline corrected of at least about 40 ng·h/mL, at least about 50 ng·h/mL, at least about 60 ng·h/mL, at least about 70 ng·h/mL, at least about 80 ng·h/mL, at least about 90 ng·h/mL, at least about 100 ng·h/mL, at least about 110 ng·h/mL, at least about 120 ng·h/mL, at least about 130 ng·h/mL.

Compositions and/or unit dosage forms as disclosed herein may provide upon administration of a single dose (or capsule) of 5 mg active agent (for example tretinoin), or one or more doses (or capsules) providing a dose equivalent to a 5 mg active agent dose, to a patient in a fasted state, a mean plasma tretinoin AUC_{0-∞} baseline corrected of between about 50 and 250 ng·h/mL, or between about 60 and 220 ng·h/mL, or between about 65 and 215 ng·h/mL, or between about 68 and 212 ng·h/mL, or between about 70 and 200 ng·h/mL, or between about 80 and 180 ng·h/mL, or between about 90 and 180 ng·h/mL, or between about 100 and 170 ng·h/mL, between about 108 and 169 ng·h/mL, or between about 110 and 150 ng·h/mL.

Compositions and/or unit dosage forms as disclosed herein may provide upon administration to a patient in a fasted state, a pharmacokinetic parameter selected from the group consisting of:
after administration to a patient in a fasted state of a single dose of a tretinoin 12 mg capsule, a mean plasma tretinoin Cₘₐₓ baseline corrected of between about 60 and 250 ng/mL;
after administration to a patient in a fasted state of a single dose of a tretinoin 12 mg capsule, a mean plasma tretinoin Cₘₐₓ baseline corrected of between about 62 and 243 ng/mL;
after administration to a patient in a fasted state of a single dose of a tretinoin 12 mg capsule, a mean plasma tretinoin Cₘₐₓ baseline corrected selected from, for example, about 60, about 62, about 62.9, about 65, about 70, about 72, about 75, about 80, about 82, about 85, about 90, about 92, about 95, about 100, about 125, about 144, about 145, about 150, about 175, about 200, about 225, about 230, about 240, about 241, about 242, about 243, about 243.3, about 244, about 245, and about 250;
after administration to a patient in a fasted state of a single dose of a tretinoin 12 mg capsule, a mean plasma tretinoin AUC_{0-∞} baseline corrected of between about 165 and 510 ng·h/mL;
after administration to a patient in a fasted state of a single dose of a tretinoin 12 mg capsule, a mean plasma tretinoin AUC_{0-∞} baseline corrected of between about 166 and 507 ng·h/mL;
after administration to a patient in a fasted state of a single dose of a tretinoin 12 mg capsule, a mean plasma tretinoin AUC_{0-∞} baseline corrected selected from, for example, about 150, about 160, about 162, about 164, about 166, about 166.1, about 170, about 175, about 200, about 225, about 250, about 275, about 300, about 323, about 325, about 350, about 375, about 400, about 425, about 450, about 475, about 500, about 501, about 502, about 503, about 504, about 505, about 506, about 506.7, about 507, about 508, about 509, and about 510.

When the capsule and/or unit dosage form is a 12 mg active agent capsule or one or more capsules providing a dose equivalent to a 12 mg active agent capsule and when the capsule is administered to a patient in a fasted state the capsule may provide at least one pharmacokinetic parameter selected from the group consisting of: a mean plasma tretinoin Cₘₐₓ baseline corrected of between about 62 and 243 ng/mL; and a mean plasma tretinoin AUC_{0-∞} baseline corrected of between about 165 and 507 ng·h/mL.

When the capsule and/or unit dosage form is a 12 mg active agent capsule or one or more capsules providing a dose equivalent to a 12 mg active agent capsule and when the capsule is administered to a patient in a fasted state the capsule may provide at least one pharmacokinetic parameter selected from the group consisting of: a mean plasma tretinoin Cₘₐₓ baseline corrected of between about 100 and 200 ng/mL; and a mean plasma tretinoin AUC_{0-∞} baseline corrected of between about 240 and 420 ng·h/mL.

When the capsule and/or unit dosage form is a 12 mg active agent capsule or one or more capsules providing a dose equivalent to a 12 mg active agent capsule and when the capsule is administered to a patient in a fasted state the capsule may provide at least one pharmacokinetic parameter selected from the group consisting of: a mean plasma tretinoin Cₘₐₓ baseline corrected of between about 115 and 180 ng/mL; and a mean plasma tretinoin AUC_{0-∞} baseline corrected of between about 258 and 404 ng·h/mL.

Compositions and/or unit dosage forms as disclosed herein may provide upon administration to a patient in a fasted state, a pharmacokinetic parameter selected from the group consisting of:
after administration to a patient in a fasted state of a single dose of a tretinoin 5 mg capsule, a mean plasma tretinoin Cₘₐₓ baseline corrected of between about 20 and 110 ng/mL;
after administration to a patient in a fasted state of a single dose of a tretinoin 5 mg capsule, a mean plasma tretinoin Cₘₐₓ baseline corrected of between about 25 and 102 ng/mL;
after administration to a patient in a fasted state of a single dose of a tretinoin 5 mg capsule, a mean plasma tretinoin Cₘₐₓ baseline corrected selected from, for example, about 25, about 26, about 26.2, about 27, about 28, about 29 about 30, about 31, about 32, about 35, about 40, about 42, about 45, about 50, about 52, about 55, about 60, about 62, about 65, about 70, about 72, about 75, about 80, about 82, about 85, about 90, about 92, about 95, about 100, about 101, about 101.4, about 102, about 103, about 104, and about 105.
after administration to a patient in a fasted state of a single dose of a tretinoin 5 mg capsule, a mean plasma tretinoin AUC_{0-∞} baseline corrected of between about 65 and about 215 ng·h/mL;
after administration to a patient in a fasted state of a single dose of a tretinoin 5 mg capsule, a mean plasma tretinoin AUC_{0-∞} baseline corrected of between about 68 and about 212 ng·h/mL;
after administration to a patient in a fasted state of a single dose of a tretinoin 5 mg capsule, a mean plasma tretinoin AUC_{0-∞} baseline corrected selected from, for example, about 68, about 69, about 69.2, about 70, about 71, about 72, about 75, about 80, about 82, about 85, about 90, about 92, about 95, about 100, about 102, about 105, about 110, about 120, about 135, about 150, about 175, about 200, about 210, about 211, about 211.1, about 212, about 213, about 214, about 215.

When the capsule and/or unit dosage form is a 5 mg active agent capsule or one or more capsules providing a dose equivalent to a 5 mg active agent capsule and when the capsule is administered to a patient in a fasted state, the capsule may provide at least one pharmacokinetic parameter selected from the group consisting of: a mean plasma tretinoin Cₘₐₓ baseline corrected of between about 25 and 102 ng/mL; and a mean plasma tretinoin AUC_{0-∞} baseline corrected of between about 68 and 212 ng·h/mL.

When the capsule and/or unit dosage form is a 5 mg active agent capsule or one or more capsules providing a dose equivalent to a 5 mg active agent capsule and when the capsule is administered to a patient in a fasted state, the capsule may provide at least one pharmacokinetic parameter selected from the group consisting of: a mean plasma tretinoin Cₘₐₓ baseline corrected of between about 40 and 90 ng/mL; and a mean plasma tretinoin AUC_{0-∞} baseline corrected of between about 90 and 180 ng·h/mL.

When the capsule and/or unit dosage form is a 5 mg active agent capsule or one or more capsules providing a dose equivalent to a 5 mg active agent capsule and when the capsule is administered to a patient in a fasted state, the capsule may provide at least one pharmacokinetic parameter selected from the group consisting of: a mean plasma tretinoin Cₘₐₓ baseline corrected of between about 48 and 75 ng/mL; and a mean plasma tretinoin AUC_{0-∞} baseline corrected of between about 108 and 169 ng·h/mL.

### Dissolution In Vitro

The dissolution of the composition and/or capsule and/or unit dosage form may also or alternatively be measured in vitro, which may provide indication of the in vivo characteristics.

The capsule and/or unit dosage form may provide release of:
of at least about 25% of the active in vitro within about 10 minutes, or at least about 30%, or at least about 35%, or at least about 40%, and/or
at least about 65% of the active in vitro within about 15 minutes, or at least about 70%, or at least about 75%, or at least about 80%, and/or
at least about 75% of the active in vitro within about 20 minutes, or at least about 80%, or at least about 85%, or at least about 90%, and/or
at least about 80% of the active in vitro within about 30 minutes, or at least about 85%, or at least about 90%, or at least about 95%, and/or
at least about 98% of the active in vitro within about 45 minutes, or at least about 99%.

Reference to release “within” a certain time point refers to measurement of the release at any point prior to and including that time point.

The capsule and/or unit dosage form may provide release of:
about 30 to 80%, or about 35 to 60%, or about 38 to 60% of the active in vitro at about 10 minutes, and/or
about 60 to 100%, or about 65 to 100%, or about 66 to 100%, of the active in vitro at about 15 minutes, and/or
about 70 to 100%, or about 75 to 100%, or about 76 to 100%, of the active in vitro at about 20 minutes, and/or
about 70 to 100%, or about 75 to 100%, or about 79 to 100%, of the active in vitro at about 30 minutes, and/or
about 75 to 100%, or about 80 to 100%, of the active in vitro at about 45 minutes.

Reference to release “at” or “after” a certain time point refers to measure of the release at the specified time (for example, measurement of the amount of release of the active at the specified time after the start of measurement in the dissolution apparatus).

In some cases, a capsule shell may delay initial release of the active when testing in vitro, for example the composition of a soft gel shell may vary the initial release of the active. However, this is not believed to substantially affect the performance of the capsule (for example the AUC_{0-∞} or Cₘₐₓ), as this type of delay is seen less when administered to a subject due to the conditions in the subject's stomach. The dissolution may therefore be measured in vitro by timing either from introduction into the dissolution testing apparatus, or from initial release of the active (for example, when release over 1% of active is seen). Timing from initial release of the active is believed to give greater understanding of the performance of the capsule fill, substantially without capsule shell effects.

The capsule and/or unit dosage form may provide release of the active agent when measured in vitro that substantially corresponds to the following dissolution pattern: (a) between about 30% and 80% of the total active agent is released after about 10 minutes of measurement in the apparatus; (b) not less than about 60% of the total active agent is released after 20 minutes of measurement in the apparatus; and (c) not less than 80% of the total active agent is released after about 30 minutes of measurement in the apparatus.

The capsule and/or unit dosage form may provide release of the active agent when measured in vitro that substantially corresponds to the following dissolution pattern: (a) between about 38 and 60% of the total active agent is released after about 10 minutes of measurement in the apparatus; (b) between about 75 and 100% of the total active agent is released after 20 minutes of measurement in the apparatus; and (c) not less than 79% of the total active agent is released after about 30 minutes of measurement in the apparatus.

The capsule and/or unit dosage form may provide release of the active agent when measured in vitro that substantially corresponds to the following dissolution pattern: (a) between about 38 and 60% of the total active agent is released after about 10 minutes of measurement in the apparatus; (b) between about 65 and 100% of the total active agent is released after 15 minutes of measurement in the apparatus; (c) not less than 75% of the total active agent is released after about 20 minutes of measurement in the apparatus.

### Carrier

The capsule comprises at least one carrier. At least one carrier may be present in the capsule fill or the liquid pharmaceutical composition.

The carrier may be a liquid. The active agent (for example tretinoin) may be substantially suspended in the carrier.

The carrier may be non-aqueous, however, it will be apparent to a person skilled in the art a non-aqueous carrier may comprise a small amount of water, for example less than about 1% w/w, or less than about 0.8% w/w, or less than about 0.5% w/w, or less than 0.3% w/w, or less than 0.25 w/w while still being considered non-aqueous.

The carrier may comprise an oil. Examples of an oil are a lipid or mineral oil (for example a light mineral oil).

Examples of lipids that may be employed include, but are not limited to, fat, oil, fatty acid, fatty acid ester, glyceride (for example a triglyceride, or medium chain triglyceride), fatty alcohol (for example C1 to C12 fatty alcohol), phospholipid, terpene, and the like, and combinations thereof.

Non-limiting examples of oils that may be employed include vegetable oil (including hydrogenated vegetable oil), for example castor oil, soybean oil, and the like or combinations thereof. Suitable hydrogenated vegetable oils that may be employed in the compositions, include but are not limited to, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, and the like, and mixtures thereof.

The fatty acid may include, but is not limited to, decanoic acid, docosanoic acid, stearic acid, palmitic acid, lauric acid, myristic acid, and the like, and mixtures thereof.

The carrier may be present in the capsule or unit dosage form in a range of about 15 to 72% w/w, or about 18 to 72% w/w, or about 18 to 68% w/w, or about 20 to 68% w/w, or about 20 to 64% w/w, or about 21 to 64% w/w, or about 21 to 60% w/w, of the capsule or unit dosage form.

The carrier may make up the largest percentage weight for weight of the components in the capsule fill. The carrier may be present in the capsule fill in a range of about 50 to 90% w/w, or about 60 to 90% w/w, or about 60 to 85% w/w, or about 65 to 85% w/w, or about 65 to 80% w/w, or about 70 to 80% w/w, or about 70 to 75% w/w, or about 50 to 74% w/w, or about 60 to 74% w/w, or about 65 to 74% w/w, or about 70 to 74% w/w, of the capsule fill.

### Viscosity Modifier

The compositions and/or unit dosage form and/or methods disclosed herein may comprise at least one viscosity modifier.

Suitable viscosity modifiers include, but are not limited to, hydrogenated vegetable oil, wax, glycerol monostearate, glyceryl behenate, magnesium aluminum silicate, propylene glycol alginate, fatty alcohol and combinations thereof.

The hydrogenated vegetable oil may be selected from hydrogenated vegetable oil Type I, hydrogenated vegetable oil Type II, or a combination thereof.

The viscosity modifier may comprise one or more waxes, for example natural waxes, (for example, animal waxes, vegetable waxes), petroleum waxes (for example, paraffin waxes, microcrystalline waxes, petrolatum waxes, mineral waxes), and/or synthetic waxes. Non-limiting examples include, but are not limited to, yellow wax, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, and the like, or mixtures thereof.

Suitable fatty alcohols that may be used as a viscosity modifier may by C12 and larger. For example, suitable fatty alcohols may include, but are not limited to cetyl alcohol, stearyl alcohol or mixtures thereof.

The viscosity modifier may have a melting point of greater than or equal to 20° C., for example, the viscosity modifier may have a melting point between about 20 and 85° C. The melting point may be measured at 1 atm.

A viscosity modifier may be present in the capsule or unit dose in a range of about 0.6 to 24% w/w, or about 0.6 to 20% w/w, or about 1.5 to 20% w/w, or about 3 to 20% w/w, or about 5 to 20% w/w, or about 5 to 16% w/w of the capsule or unit dosage form.

A viscosity modifier may be present in the capsule fill in a range of about 2 to 30% w/w, or about 2 to 25% w/w or about 5 to 25% w/w, or about 10 to 25% w/w, or about 15 to 25% w/w, or about 15 to 20% w/w of the capsule fill.

### Chelating Agent

The term “chelating agent”, as used herein, means a molecule containing two or more electron donor atoms that can form coordinate bonds to a single metal ion. The term “chelating agent” is understood to include the chelating agent as well as salts and/or hydrates thereof. For example, the term “chelating agent” includes citric acid as well as its salt forms and/or hydrates (for example citric acid monohydrate).

The most common and widely used chelating agents coordinate to metal atoms through oxygen or nitrogen donor atoms, or both. Other less common chelating agents coordinate through sulfur in the form of —SH (thiol or mercapto) groups. After the first coordinate bond is formed, each successive donor atom that binds may create a ring or cluster containing the metal atom. A chelating agent may be bidentate, tridentate, tetradentate, etc., depending upon whether it contains two, three, four, or more donor atoms capable of binding to the metal atom. See Kirk-Othmer Encyclopedia of Chemical Technology (4^{th} ed. 2001).

The compositions and/or unit dosage form and/or methods disclosed herein may comprise at least one chelating agent.

The chelating agent may be, for example, ethylenediamine tetraacetic acid (EDTA), diethylenetriaminepentaacetic acid (DTPA), hydroxyethylethylenediaminetriacetic acid (HEDTA), malic acid, citric acid or a pharmaceutically acceptable salt (for example disodium edetate (disodium EDTA)) or hydrate thereof, and combinations thereof.

The amount of chelating agent present in the compositions and/or methods disclosed herein will depend on the particular chelating agent or agents (i.e. mixtures of chelating agents) selected, and/or the amount of active agent present in the capsule.

The chelating agent may be present in the capsule or unit dosage form in a range of about 0.003 to 8% w/w, or about 0.003 to 4% w/w, or about 0.003 to 2.4% w/w, or about 0.003 to 1.6% w/w, or about 0.003 to 0.8% w/w, or about 0.01 to 0.8% w/w, or about 0.03 to 0.8% w/w, or about 0.03 to 0.6% w/w, of the capsule or unit dosage form.

The chelating agent may be present in the capsule fill in a range of about 0.01 to 10% w/w, or about 0.01 to 5% w/w, or about 0.01 to 3% w/w, or about 0.01 to 2% w/w, or about 0.01 to 1% w/w, or about 0.05 to 1% w/w, or about 0.1 to 1% w/w, or about 0.1 to 0.8% w/w of the capsule fill.

The composition/unit dosage form/method as disclosed herein may contain a safe and effective amount of a chelating agent suitable for achieving the desired chelating effect. In at least one example, the capsule may contain about 0.1 mg, about 0.2 mg, about 0.3 mg, about 0.4 mg, about 0.5 mg, about 0.6 mg, about 0.7 mg, about 0.8 mg, about 0.9 mg, or about 1.0 mg per unit dose (for example each capsule) of disodium EDTA.

### Antioxidant

The compositions and/or unit dosage form and/or methods may comprise one or more antioxidant(s) to inhibit the oxidation of ingredients. The capsule fill may comprise one or more antioxidant(s).

Some examples of antioxidants include but are not limited to, butylated hydroxyanisole (BHA) (for example 2-tert-butyl-4-methoxyphenol and/or 3-tert-butyl-4-methoxyphenol), butylated hydroxytoluene (BHT), propyl gallate, tocopherols and/or tocotrienols and mixture and/or derivatives (for example, vitamin E, α-tocopherol), propionic acid, sodium nitrate, sodium nitrite, citric acid (for example citric acid monohydrate), 4-chloro-2,6-ditertiary butylphenol, dihydroguaretic acid, potassium sorbate, sodium bisulfate, sodium bisulfite, sodium metabisulfite, potassium metabisulfite, sorbic acid, malic acid, fumaric acid, ascorbic acid (vitamin C) and its derivatives (such as sodium ascorbate, potassium ascorbate, sodium isoascorbate, ascorbyl palmitate), and combinations thereof.

The antioxidant may be selected from the group consisting of butylated hydroxyanisole (BHA), butylated hydroxytoluene (BHT), propyl gallate, tocopherols (e.g., α-tocopherol (vitamin E)), propionic acid, sodium nitrate, sodium nitrite, citric acid (for example citric acid monohydrate) and combinations thereof.

The compositions and/or methods may comprise more than one antioxidant, for example a combination of 2, 3, or more antioxidants, for example a combination of butylated hydroxyanisole and/or butylated hydroxytoluene and vitamin E.

The antioxidant may be present in the capsule or unit dosage form in a range of about 0.0003 to 8%, or about 0.003 to 4% w/w of the capsule fill, or about 0.003 to 2.5% w/w, of the capsule or unit dosage form.

The antioxidant may be present in the capsule fill in a range of about 0.001 to 10%, or about 0.01 to 5% w/w of the capsule fill, or about 0.01 to 3% w/w, of the capsule fill.

The amount of the antioxidant used will vary depending on the antioxidant used. For example, antioxidants such as BHA and/or BHT may each be present in an amount of from about 0.01 to 0.50% w/w based upon the total weight of the capsule fill, e.g., about 0.01 to 0.35% w/w or about 0.01 to 0.1% w/w. For example, vitamin E may be present in the capsule fill in an amount of about 0.1 to 5% w/w, or about 0.5 to 3% w/w, of the capsule fill.

The compositions and/or methods and/or unit dosage form may comprise the active agent in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form, the carrier in a range of about 15 to 72% w/w of the capsule or unit dosage form, and the viscosity modifier in a range of about 0.6 to 20% w/w of the capsule or unit dosage form.

Alternatively, the compositions and/or methods and/or unit dosage form may comprise the active agent in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form, the carrier in a range of about 15 to 72% w/w of the capsule or unit dosage form, the viscosity modifier in a range of about 0.6 to 20% w/w of the capsule or unit dosage form, and the antioxidant in a range of about 0.003 to 8% w/w of the capsule or unit dosage form.

Alternatively, the compositions and/or methods and/or unit dosage form may comprise the active agent in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form, the carrier in a range of about 15 to 72% w/w of the capsule or unit dosage form, the viscosity modifier in a range of about 0.6 to 20% w/w of the capsule or unit dosage form, the chelating agent in a range of about 0.003 to 8% w/w of the capsule or unit dosage form, and the antioxidant in a range of about 0.003 to 8% w/w of the capsule or unit dosage form.

Alternatively, the compositions and/or methods and/or unit dosage form may comprise the active agent in a range of about 0.6 to 24% w/w of the capsule or unit dosage form, the carrier in a range of about 15 to 72% w/w of the capsule or unit dosage form, the viscosity modifier in a range of about 0.6 to 20% w/w of the capsule or unit dosage form, the chelating agent in a range of about 0.003 to 8% w/w of the capsule or unit dosage form, and the antioxidant in a range of about 0.003 to 8% w/w of the capsule or unit dosage form.

The compositions and/or methods and/or unit dosage form may comprise the active agent in a range of about 1 to 55% w/w, or about 2 to 50% w/w, or about 2 to 40% w/w, or about 2 to 30% w/w, or about 2 to 20% w/w, or about 5 to 20% w/w of the capsule fill, the carrier in a range of about 50 to 90% w/w of the capsule fill, the viscosity modifier is present in a range of about 2 to 25% w/w of the capsule fill.

Alternatively, the compositions and/or methods and/or unit dosage form may comprise the active agent in a range of about 1 to 55% w/w, or about 2 to 50% w/w, or about 2 to 40% w/w, or about 2 to 30% w/w, or about 2 to 20% w/w, or about 5 to 20% w/w of the capsule fill; the carrier in a range of about 50 to 90% w/w of the capsule fill; the viscosity modifier in a range of about 10 to 25% w/w of the capsule fill.

Alternatively, the compositions and/or methods and/or unit dosage form may comprise the active agent in a range of about 1 to 55% w/w, or about 2 to 50% w/w, or about 2 to 40% w/w, or about 2 to 30% w/w, or about 2 to 20% w/w, or about 5 to 20% w/w of the capsule fill, the carrier in a range of about 50 to 90% w/w of the capsule fill, the viscosity modifier in a range of about 2 to 25% w/w of the capsule fill, and the antioxidant in a range of about 0.01 to 10% w/w of the capsule fill.

Alternatively, the compositions and/or methods and/or unit dosage form may comprise the active agent in a range of about 1 to 55% w/w, or about 2 to 50% w/w, or about 2 to 40% w/w, or about 2 to 30% w/w, or about 2 to 20% w/w, or about 5 to 20% w/w of the capsule fill; the carrier in a range of about 50 to 90% w/w of the capsule fill; the viscosity modifier in a range of about 10 to 25% w/w of the capsule fill; the antioxidant in a range of about 0.01 to 10% w/w of the capsule fill.

Alternatively, the compositions and/or methods and/or unit dosage form may comprise the active agent in a range of about 1 to 55% w/w, or about 2 to 50% w/w, or about 2 to 40% w/w, or about 2 to 30% w/w, or about 2 to 20% w/w, or about 5 to 20% w/w of the capsule fill, the carrier in a range of about 50 to 90% w/w of the capsule fill, the viscosity modifier in a range of about 2 to 25% w/w of the capsule fill, the chelating agent in a range of about 0.01 to 10% w/w of the capsule fill, and the antioxidant in a range of about 0.01 to 10% w/w of the capsule fill.

Alternatively, the compositions and/or methods and/or unit dosage form may comprise the active agent in a range of about 1 to 55% w/w, or about 2 to 50% w/w, or about 2 to 40% w/w, or about 2 to 30% w/w, or about 2 to 20% w/w, or about 5 to 20% w/w of the capsule fill; the carrier in a range of about 50 to 90% w/w of the capsule fill; the viscosity modifier in a range of about 10 to 25% w/w of the capsule fill; the chelating agent in a range of about 0.01 to 10% w/w of the capsule fill; the antioxidant in a range of about 0.01 to 10% w/w of the capsule fill.

Alternatively, the compositions and/or methods and/or unit dosage form may comprise the active agent in a range of about 2 to 30% w/w of the capsule fill, the carrier in a range of about 50 to 90% w/w of the capsule fill; the viscosity modifier in a range of about 2 to 25% w/w of the capsule fill; the chelating agent in a range of about 0.01 to 10% w/w of the capsule fill, the antioxidant in a range of about 0.01 to 10% w/w of the capsule fill.

Alternatively, the compositions and/or methods and/or unit dosage form may comprise the active agent in a range of about 2 to 30% w/w of the capsule fill; the carrier in a range of about 50 to 90% w/w of the capsule fill; the viscosity modifier in a range of about 10 to 25% w/w of the capsule fill; the chelating agent in a range of about 0.01 to 10% w/w of the capsule fill; the antioxidant in a range of about 0.01 to 10% w/w of the capsule fill

The capsule and/or capsule fill may contain less than about 5% w/w, or less than about 2% w/w, less than about 1% w/w, or less than about 0.5% w/w, or less than about 0.2% w/w, less than about 0.15% w/w, less than about 0.1% w/w isotretinoin based on the weight of the active agent.

The capsule and/or capsule fill may contain less than about 5% w/w, or less than 2% w/w, or less than about 1% w/w, or less than about 0.5% w/w, or less than about 0.2% w/w, less than about 0.15% w/w isotretinoin based on the weight of the active agent after being stored at about 25 to 30° C. in an opaque container for about 3 months, or about 6 months, or about 9 months, or about 12 months, or about 18 months, or about 24 months, or about 36 months. The capsule and/or capsule fill may contain less than about 0.1% w/w isotretinoin based on the weight of the active agent after being stored at about 25 to 30° C. in an opaque container for about 3 months, or about 6 months, or about 9 months, or about 12 months, or about 18 months, or about 24 months.

The capsule may be stored at a relative humidity of 60% or 75%, or 60% at 25° C., or 75% at 30° C.

### Capsule Shell

A capsule may be a hard or soft gelatin capsule, a starch capsule, a cellulosic capsule, preferably a soft gelatin capsule.

The capsule shell is the outer shell that encapsulates the capsule fill. The capsule shell may be a hard or soft gelatin, a starch, or cellulosic, preferably soft gelatin.

The capsule fill may be about 30 to 80% w/w of the capsule (based on the shell dry weight). The capsule shell (dry weight) may be about 20 to 70% w/w, or about 30 to 60% w/w of the capsule. The thickness and/or size of the capsule shell may be selected depending on the size of the capsule.

When the capsule and/or unit dosage form is a 12 mg active agent capsule, the capsule shell dry weight may be about 30 to 50% w/w of the capsule, or the capsule shell dry weight may be about 40 to 45% w/w of the capsule.

When the capsule and/or unit dosage form is a 5 mg active agent capsule, the capsule shell dry weight may be about 40 to 60% w/w of the capsule, or the capsule shell dry weight may be about 50 to 60% w/w of the capsule.

The shell may contain a gelling agent such as animal or fish protein (gelatin) or plant polysaccharides or their derivatives such as carrageenans and modified forms of starch and cellulose. Along with the gelling agent, the capsule shell may also include plasticizers (such as those mention below (e.g. glycerol, sorbitol, propylene glycol) and water.

The gelatin may be present in the capsule in a range of about 20 to 80% w/w of the capsule shell. The percentage will in part be dependent on whether the wet mass of dry mass is measured. The gelatin may be present in the capsule in a range of about 20 to 80% w/w of the wet mass of the capsule shell.

Water may be present in the capsule in a range of about 10 to 50% w/w of the wet mass of the capsule shell.

Plasticizers can also be included in the compositions and/or methods and/or unit dosage form as disclosed herein, in particular, for example, in the capsule shell.

As used herein, the term “plasticizer” includes all compounds capable of plasticizing or softening a polymer or binder used in invention. The plasticizer should be able to lower the melting temperature or glass transition temperature (softening point temperature) of the polymer or binder. Plasticizers, such as low molecular weight PEG, generally broaden the average molecular weight of a polymer in which they are included thereby lowering its glass transition temperature or softening point. Plasticizers also generally reduce the viscosity of a polymer. It is possible the plasticizer will impart some particularly advantageous physical properties to the osmotic device of the invention.

Plasticizers useful in the compositions and/or methods and/or unit dosage form as disclosed herein can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Plasticizers used for soft gelatin capsules include polyalcohols such as glycerol, propylene glycol, polyethylene glycol, sorbitol, sorbitol/sorbitan and mannitol and combinations thereof. Other such plasticizers can also include ethylene glycol, 1,2-butylene glycol, 2,3-butylene glycol, styrene glycol, diethylene glycol, diethyl phthalate, dimethyl phthalate, butyl and glycol esters of fatty acids, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyl tributyl citrate, triethyl citrate, acetyltriethyl citrate, tributyl citrate, diacetin, triacetin, benzyl benzoate, dextrin, acetylated monoglyceride, refined mineral oils, oleic acid, stearic acid, palmitic acid, ethyl alcohol, stearyl alcohol, pyrrolidone, castor oil, corn oil, camphor, allyl glycolate and combinations thereof.

The PEG based plasticizers are available commercially or can be made by a variety of methods, such as disclosed in Poly(ethylene glycol) Chemistry: Biotechnical and Biomedical Applications (J. M. Harris, Ed.; Plenum Press, NY) the disclosure of which is hereby incorporated by reference.

All such plasticizers are commercially available from sources such as Aldrich or Sigma Chemical Co. It is also contemplated and within the scope of the invention, that a combination of plasticizers may be used in the present compositions and/or methods and/or unit dosage form.

The plasticizer may be present in a concentration of about 0.5% to 50% w/w, or about 1 to 40% w/w, or about 1 to 30% w/w or about 1 to 25% w/w of the composition, such as in the capsule shell. In at least one example, the plasticizer is present in the capsule shell at a concentration of about 1% to 50% w/w, or about 1 to 40% w/w, or about 1 to 30% w/w or about 1 to 25% w/w of the capsule shell, based on wet mass of the capsule shell. In at least one example, the plasticizer is present in the capsule shell in a concentration of about 10 to 30% w/w of the capsule shell, based on wet mass of the capsule shell.

The pharmaceutical compositions (either capsule shell and/or capsule fill) and/or methods and/or unit dosage form may further comprise one or more additional materials such as a pharmaceutically compatible carrier, binder, filling agent, suspending agent, flavoring agent, sweetening agent, disintegrating agent, surfactant, preservative, lubricant, colorant, diluent, solubilizer, moistening agent, stabilizer, wetting agent, anti-adherent, anti-foaming agent, antifungal agent, antibacterial agent, pH modifier or one or more combination thereof.

The invention will be illustrated in more detail with reference to the following Examples, but it should be understood that the present invention is not deemed to be limited thereto.

### EXAMPLES

### Example 1

Table 1 describes examples of fill formulations for capsule with 5 mg, 10 mg, 12 mg, 15 mg and 20 mg of tretinoin.

TABLE 1
Formulation Examples 1-5 - Capsule Fill Formulations

| | Example 1 | Example 2 | Example 3 | Example 4 | Example 5 |
|---|---|---|---|---|---|
| | (5 mg) | (10 mg) | (12 mg) | (15 mg) | (20 mg) |
| Capsule Fill | mg/cap | mg/cap | mg/cap | mg/cap | mg/cap |
| ingredient | (% w/w) | (% w/w) | (% w/w) | (% w/w) | (% w/w) |
| Tretinoin | 5.000 | 10.000 | 12.000 | 15.000 | 20.000 |
| | (7.5) | (7.5) | (7.5) | (7.5) | (7.5) |
| Soybean oil | 48.963 | 97.927 | 117.513 | 146.890 | 195.854 |
| | (73.4) | (73.4) | (73.4) | (73.4) | (73.4) |
| Hydrogenated | 1.967 | 3.933 | 4.720 | 5.900 | 7.867 |
| vegetable oil, | (3.0) | (2.9) | (3.0) | (3.0) | (3.0) |
| type I | | | | | |
| Hydrogenated | 7.867 | 15.733 | 18.880 | 23.600 | 31.466 |
| vegetable oil, | (11.8) | (11.8) | (11.8) | (11.8) | (11.8) |
| type II | | | | | |
| Yellow Wax | 1.967 | 3.933 | 4.720 | 5.900 | 7.867 |
| | (3.0) | (2.9) | (3.0) | (3.0) | (3.0) |
| Disodium Edetate | 0.192 | 0.383 | 0.460 | 0.575 | 0.767 |
| | (0.3) | (0.3) | (0.3) | (0.3) | (0.3) |
| Butylated | 0.042 | 0.083 | 0.100 | 0.125 | 0.167 |
| Hydroxyanisole | (0.06) | (0.06) | (0.06) | (0.06) | (0.06) |
| (BHA) | | | | | |
| Vitamin E | 0.669 | 1.339 | 1.607 | 2.008 | 2.678 |
| | (1.0) | (1.0) | (1.0) | (1.0) | (1.0) |
| Total Capsule Fill | 66.666 mg | 133.332 mg | 160.000 mg | 199.998 mg | 266.664 mg |
| Particle Size | 1 to 5 μm | | | | |
| D[v, 0.1] μm | | | | | |
| Particle Size | 5 to 10 μm | | | | |
| D[v, 0.5] μm | | | | | |
| Particle Size | 10 to 20 μm | | | | |
| D[v, 0.9] μm | | | | | |

Hydrogenated vegetable oil type 1 and type 2 are mixtures of fatty acids and are differentiated by the melting temperature. Type 1 is solid in nature, whereas type 2 is unctuous in nature.

Table 2 from the US Pharmacopeia (USP) provides physical data for the two types.

TABLE 2
Physical data for Type I and Type II hydrogenated vegetable oil

| | Type I | Type II |
|---|---|---|
| Melting range, Class II <741> | 57 to 85° C. | 20 to 50° C. |
| Heavy metals, Method II <231> | 0.001% | 0.001% |
| Iodine value, Method II <401> | 0 to 5 | 55 to 80 |
| Saponification value <401> | 175 to 200 | 175 to 200 |

Table 3 describes an example of a soft gel capsule shell formulation. The amount of capsule shell varies between size/dose of capsule, for example the capsule with 12 mg of tretinoin (Example 3) has a wet shell mass of about 170 mg and dry shell mass of about 120 mg, the capsule with 5 mg of tretinoin (Example 1) has a wet shell mass of about 120 mg and dry shell mass of about 86 mg.

TABLE 3
Example of shell formulation

| | | Example 3 |
|---|---|---|
| | | (12 mg) |
| Capsule Shell ingredient | % w/w | mg/cap |
| Gelatin | 44.202 | 75.144 |
| Glycerol | 11.321 | 19.246 |
| Sorbitol 70% Non-Crystalline | 10.090 | 17.153 |
| Yellow, Iron oxide | 0.095 | 0.162 |
| Titanium dioxide | 0.999 | 1.698 |
| Purified water | 33.289 | 56.598 |
| Total Capsule Shell (wet mass) | 100.00% | 170.00 mg |
| Total Capsule Shell (dry mass) | | About 120 mg |

Table 4 describes the function of the ingredients for the Tretinoin capsules example formulations.

TABLE 4
Function of ingredients from above Examples

| | | |
|---|---|---|
| | Quality | |
| Ingredient | Standard | Function |

Capsule Fill

| | | |
|---|---|---|
| Tretinoin | USP | Active Pharmaceutical |
| | | Ingredient (API) |
| Soybean Oil | USP | Carrier |
| Yellow Wax (beeswax) | NF | Viscosity Modifier |
| Hydrogenated Vegetable Oil, | NF | Viscosity Modifier |
| Type I | | |
| Hydrogenated Vegetable Oil, | NF | Viscosity Modifier |
| Type II | | |
| Butylated Hydroxyanisole | NF | Antioxidant |
| Edetate Disodium | USP | Antioxidant/ |
| | | Chelating Agent |
| Vitamin E | USP | Antioxidant |

Capsule Shell

| | | |
|---|---|---|
| Gelatin | NF | Matrix |
| Glycerin ^{a} | USP | Plasticizer |
| Non-crystallizing Sorbitol Solution | NF | Plasticizer |
| Ferric Oxide, Yellow | NF | Colorant |
| Titanium Dioxide | USP | Opacifier |
| Purified Water | USP | Processing Aid |

^{a} Glycerin and glycerol are synonyms of the same material.

### Example 2—Manufacturing Method

### Capsule Fill

An example of a method of making the capsule fills of the above Formulation Examples is using the following steps:
1. Soybean oil, Hydrogenated Vegetable Oil Type I, Hydrogenated Vegetable Oil Type II, Yellow Wax and BHA were mixed in a stainless-steel vessel while heating to about 65 to 75° C. (to make liquid) with scraper on. On reaching temperature with the scraper and dispersion disk on the components were mixed until the solution was substantially clear and free of particles.
2. After cooling, disodium edetate, vitamin E and tretinoin were added and mixed with the scraper and dispersion disk on until substantially no lumps were present.
3. The suspension was bead milled with 0.4-0.6 mm beads until a D[v, 0.9] of between 10 to 20 μm was reached, while maintaining the temperature <40° C.
4. The milled suspension was passed through a deaerator until no air bubbles were present. The milled suspension was then transferred to a nitrogen-purged medicine holding vessel prior to encapsulation. If required, the milled suspension was remixed prior to encapsulation.

### Soft Gel Capsule Shell

An example of a method of making a soft gel capsule is using the following steps:
1. Purified water, sorbitol solution (non-crystallizing) and glycerol were mixed in a gelatin melter at about 75 to 80° C.
2. Granulated gelatin was added and mixed at about 65 to 75° C. for about 60 to 80 minutes under vacuum to melt.
3. The clear gelatin mass was degassed and sieved through a 400 μm sieve into a preheated holding tank at about 60° C.
4. Titanium dioxide, yellow iron oxide, glycerol and purified water were mixed until completely dispersed then passed through a colloid mill gap setting 30. When homogeneous and lump free the color paste was mixed into the gelatin mass at 600 to 800 rpm for 20 minutes.
5. The colored gelatin mass was stored (if necessary) at approximately 60° C. prior to encapsulation.

### Encapsulation

An example of a method of encapsulating the capsule fills is using the following steps:
1. The gelatin ribbon lubricant reservoir was filled with medium chain triglycerides in sufficient quantity for the encapsulation run.
2. The external capsule shell lubricant (e.g. 8% Phosal 53 in Captex 355) was sprayed onto the capsule chute surfaces and polishing cloths, until adequately moistened.
3. The coloured gelatin mass and the milled suspension were brought together using encapsulation rotary die roll.
4. The capsules were dried.
5. The dried capsules were inspected and sorted to reject any oddly shaped formed capsules.
6. The capsules were polished.
7. The capsules were then packed.

The methods above produce yellow, oval, soft gelatin capsules, containing a yellow to orange, opaque, viscous suspension.

A fill formulation using 27.4% w/w viscosity modifier (combination of hydrogenated vegetable oil, type I and type II and yellow wax) was tried, but gave a very thick viscous fill mixture following milling, which was not suitable for encapsulation in a shell.

### Example 3—Stability Studies

Stability studies were preformed to confirm the stability of the fill formulation, shell and/or capsule. The stability of the 12 mg and 5 mg capsule Examples were tested. The results are shown in Tables 5 and 6.

A description of the appearance of the capsules was recorded at each measure. The appearance remained the same at all data points for both the 5 mg and 12 mg capsules, i.e. yellow, oval, soft gelatin capsule containing a yellow to orange, opaque, viscous suspension.

The results in Tables 5 and 6 show both the 5 mg and 12 mg capsules have good stability.

The percentage of the active tretinoin is consistent over the time of the study. This is despite the small particle size.

The levels of the isomer isotretinoin are relatively stable. At all times during the study, isotretinoin is less than 5% w/w (and/or less than 0.2% w/w) of the active, showing there is not significant isomerization.

The particle size is also consistent over time for both capsules. A concern when dealing with low particle sizes is clumping. This can lead to increasing particle size, which can affect the pharmacokinetics. However, this was not evident in the stability studies.

The particle distribution D[v,0.5] remains within the range about 5 to 8 μm for either capsule. The particle distribution D[v,0.5] remains about 5 μm for the 5 mg capsule. The particle distribution D[v,0.5] remains about 6 to 8 μm for the 12 mg capsule.

The particle size distribution D[v,0.1] remains greater than about 1 μm (about 2 μm) for either capsule.

The particle size distribution D[v,0.9] remains not more than 20 μm or about 11 to 18 μm for either capsule. The particle distribution D[v,0.9] remains about 11 to 13 μm for the 5 mg capsule. The particle distribution D[v,0.9] remains about 15 to 18 μm for the 12 mg capsule.

TABLE 5
Stability results 5 mg capsules
Tretinoin 5 mg Capsules
Packaging: BOT 75 ml HOPE WHT, CAP 38 mm PP WHT CRC FOIL WAD;
Storage Conditions: 30° C./75% RH

| | | Months | | | | | | | |
|---|---|---|---|---|---|---|---|---|---|
| Test | Specifications | 0 | | 3 | | 6 | | 9 | |
| Tretinoin UHPLC | 90.0 to 110.0% | 100.2, 97.6 | | 94.8, 94.1 | | 96.3, 96.0 | | 97.2, 97.8 | |
| (% of labelled | | Average = 98.9 | | Average = 94.5 | | Average = 96.2 | | Average = 97.5 | |
| content) | | | | | | | | | |
| Dissolution UV | Not less than 75% (Q) | 97 | | 95 | | 95 | | 95 | |
| (Mean %) | of the labelled amount | | | | | | | | |
| | is dissolved in 45 | | | | | | | | |
| | minutes | | | | | | | | |
| Related Substances UHPLC | | Prep 1 | Prep 2 | Prep 1 | Prep 2 | Prep 1 | Prep 2 | Prep 1 | Prep 2 |
| Isotretinoin | Not more than 5.0% of | 0.07 | 0.07 | 0.02 | 0.03 | 0.04 | 0.04 | 0.11 | 0.09 |
| (% w/w) | the tretinoin | | | | | | | | |
| Total | Not more than 1.5% of | 0.08 | 0.08 | 0.08 | 0.08 | 0.08 | 0.08 | 0.08 | 0.08 |
| Unspecified | the tretinoin | | | | | | | | |
| Impurities | | | | | | | | | |
| (% w/w) | | | | | | | | | |

Particle size

| | | | | |
|---|---|---|---|---|
| D[v, 0.1] (μm) | 2 | 2 | 2 | 2 |
| D[v, 0.5] (μm) | 5 | 5 | 5 | 5 |
| D[v, 0.9] (μm) | 12 | 12 | 11 | 13 |

TABLE 6
Stability results 12 mg capsules
Tretinoin 12 mg Capsules
Packaging: 100 ml HOPE white plastic bottles, induction sealed with child resistant caps (38 mm);
Storage Conditions: 25° C./60% RH

| | | Months | | | | | | | |
|---|---|---|---|---|---|---|---|---|---|
| Test | Specifications | 0 | 3 | 6 | 9 | 12 | 18 | 24 | 36 |
| Tretinoin | 90.0 to 110.0% | 101.3, 99.7 | 101.0, 101.0 | 100.2, 101.0 | 100.2, 100.5 | 101.1, 102.4 | 100.4, 100.1 | 99.5, 101.5 | 101.6, 101.8 |
| UHPLC | | Ave = 100.5 | Ave = 101.0 | Ave = 100.6 | Ave = 100.4 | Ave = 101.8 | Ave = 100.3 | Ave = 100.5 | Ave = 101.7 |
| (% of labelled | | | | | | | | | |
| content) | | | | | | | | | |
| Dissolution UV | Not less than | 99 | 95 | 98 | 100 | 101 | 99 | 99 | 100 |
| (Mean %) | 75% (Q) of the | | | | | | | | |
| | labelled amount is | | | | | | | | |
| | dissolved in 45 | | | | | | | | |
| | minutes | | | | | | | | |

Related Substances UHPLC

| | | | | | | | | | |
|---|---|---|---|---|---|---|---|---|---|
| Isotretinoin (% | Not more than | 0.05 | 0.05 | 0.05 | 0.03 | 0.07 | 0.06 | 0.02 | 0.14 |
| w/w) | 5.0% of the | | | | | | | | |
| | tretinoin | | | | | | | | |
| Total | Not more than | 0.11 | 0.12 | 0.16 | 0.10 | 0.19 | 0.13 | 0.09 | 0.21 |
| Unspecified | 1.5% of the | | | | | | | | |
| Impurities (% | tretinoin | | | | | | | | |
| w/w) | | | | | | | | | |

Particle size - Laser Diffraction

| | | | | | | | | |
|---|---|---|---|---|---|---|---|---|
| D[v, 0.1] (μm) | 2 | 2 | 2 | 2 | 2 | 2 | 2 | 2 |
| D[v, 0.5] (μm) | 6 | 7 | 7 | 7 | 7 | 7 | 8 | 7 |
| D[v, 0.9] (μm) | 15 | 16 | 16 | 16 | 16 | 16 | 17 | 18 |

### Example 4—Dissolution Testing

The dissolution profile of the of the Test 12 mg Tretinoin capsule (Formulation Example 3, 12 mg capsules) was measured under the conditions shown in Table 7.

TABLE 7
Dissolution testing conditions

| | |
|---|---|
| Dissolution Medium: | 0.05M phosphate buffer pH 7.8 with 0.5% LDAO |
| Sample Volume | 20 mL |
| Medium Quantity: | 900 mL per vessel |
| Apparatus: | USP Apparatus II (Paddles) |
| Temperature | 37.0 ± 0.5° C. |
| Speed: | 100 rpm ± 4 rpm |
| Sinker | Spiral capsule sinker, 316 SS, 0.84 inches |
| | (L) × 0.385 inches (W) capacity, 5 coils, |
| | Part# CAPWHT -4S (or equivalent) |

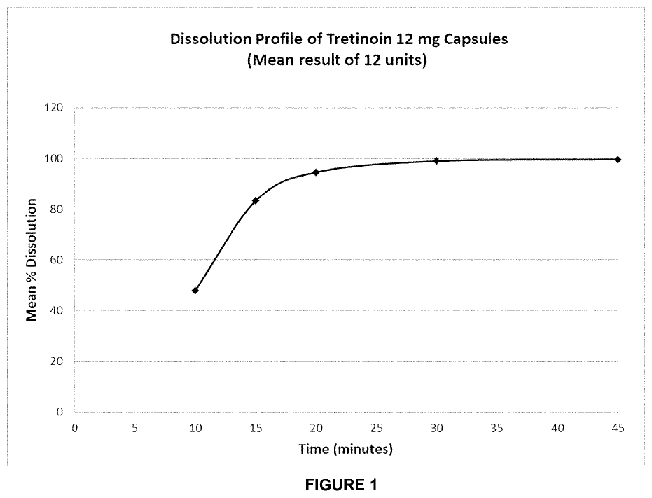

12 samples (capsules) of the Test 12 mg Tretinoin capsules were measured separately and the mean dissolution profile calculated. The results are shown in Table 8. The mean % dissolution for the Test 12 mg Tretinoin capsules is shown in FIG. 1.

TABLE 8
Dissolution profiles for Test 12 mg Tretinoin capsules

| Time | Mean % dissolution | Std Dev | % CV |
|---|---|---|---|
| 10 | 48 | 30.5 | 63.7 |
| 15 | 83 | 22.9 | 27.5 |
| 20 | 95 | 10.9 | 11.5 |
| 30 | 99 | 1.4 | 1.5 |
| 45 | 100 | 2.2 | 2.2 |

### Example 5—Fed Bioavailability Study

A study was completed in New Zealand to compare the bioavailability of tretinoin for 1×12 mg Tretinoin capsule (Formulation Example 3), relative to that of a Reference formulation, (2×10 mg Tretinoin capsules, Glenmark Pharmaceuticals, USA), in healthy, adult, male subjects under fed conditions in a single-centre, single-dose, two-treatment, two-period, two-sequence, two-way crossover, randomized study.

Each subject was randomly assigned to one of the treatment sequences shown in Table 9. There was a washout period of at least 2 weeks between treatments. Eighteen subjects provided at least one post-dosing blood sample. The 18 male subjects had a mean (range) age of 24 (19-46) years, a mean (range) weight of 78.4 (67.7-102.6) kg, a mean (range) height of 1.82 (1.70-1.93) m and a mean (range) BMI of 23.6 (19.6-29.9) kg/m². There were 16 (88.89%) subjects who were White, 1 (5.56%) subject who was Other Pacific Islander and 1 (5.56%) subject who was White/Other Pacific Islander. Fifteen subjects completed the study in accordance with the Protocol.

TABLE 9
Treatment sequences

| | Period 1 | Period 2 |
|---|---|---|
| Sequence 1 | Test (1 × 12 mg capsule | Reference (2 × 10 mg |
| | tretinoin - Formulation | capsules, commercially |
| | Example 3) | available, Glenmark) |
| Sequence 2 | Reference (2 × 10 mg | Test (1 × 12 mg capsule |
| | capsules, commercially | tretinoin - Formulation |
| | available, Glenmark) | Example 3) |

Subjects attended the Clinical Site the evening prior to dosing and fasted overnight for at least 10 hours. Subjects were randomly assigned to one of the two study treatments.

The treatments were administered after pre-dose clinical assessments were complete and baseline blood samples (−1, −0.5 and 0 hours) were collected. The treatments were administered orally with 240 ml of ambient-temperature water. The capsules were swallowed whole.

Subjects consumed a standardized high fat meal within 30-minutes prior to dosing (approx. 863 calories, with approx. 30.5 g protein (approx. 122 calories, approx. 14.1% of total calories), approx. 63.2 g carbohydrate (approx. 253 calories, approx. 29% of total calories), approx. 53.3 g fat (approx. 480 calories, approx. 56% of total calories)). The standardized high-fat meal composition is shown in Table 10. Note that all values are approximate.

TABLE 10
Standardized high-fat meal composition

| | | 2 × | | | | 4 oz | | |
|---|---|---|---|---|---|---|---|---|
| | | eggs | 2 × | 2 × | | hash | 200 ml | |
| | | fried in | strips | slices | 20 g | brown | whole | |
| | | butter | bacon | toast | butter | potatoes | milk | Total |
| Calories | Approx. | 180 | 84 | 128 | 104 | 218 | 149 | 863 |
| | Total | | | | | | | |
| Protein | Grams | 11.6 | 6.1 | 4.0 | 0.1 | 0.7 | 8.0 | 30.5 |
| | Calories | 46.4 | 24.2 | 15.8 | 0.5 | 2.8 | 31.9 | 121.6 |
| | Rel. cal. % | 25.8 | 28.9 | 12.4 | 0.5 | 1.3 | 21.4 | 14.1 |
| Carbo- | Grams | 1.2 | 0.2 | 23.9 | 0.0 | 26.7 | 11.2 | 63.2 |
| hydrates | Calories | 4.8 | 1.0 | 95.8 | 0.0 | 106.8 | 44.7 | 253.1 |
| | Rel. cal. % | 2.7 | 1.1 | 74.8 | 0.0 | 49.0 | 30.0 | 29.3 |
| Fat | Grams | 14.1 | 6.4 | 1.8 | 11.5 | 11.5 | 8.0 | 53.3 |
| | Calories | 126.0 | 58.0 | 16.0 | 103.7 | 104.0 | 72.0 | 479.7 |
| | Rel. cal. % | 70.0 | 69.0 | 12.5 | 99.7 | 47.7 | 48.3 | 55.6 |

Subjects remained at the Clinical Site for 12 hours after dosing for the collection of blood samples (8 ml) through venous catheters at: 0.25, 0.5, 0.75, 1, 1.25, 1.5, 1.75, 2, 2.5, 3, 3.5, 4, 4.5, 5, 5.5, 6, 6.5, 7, 7.5, 8, 9, 10, 11 and 12 hours. Subjects were required to fast for at least 4 hours after the drug administration. Subjects were released from the Clinical Site following collection of the 12-hour sample.

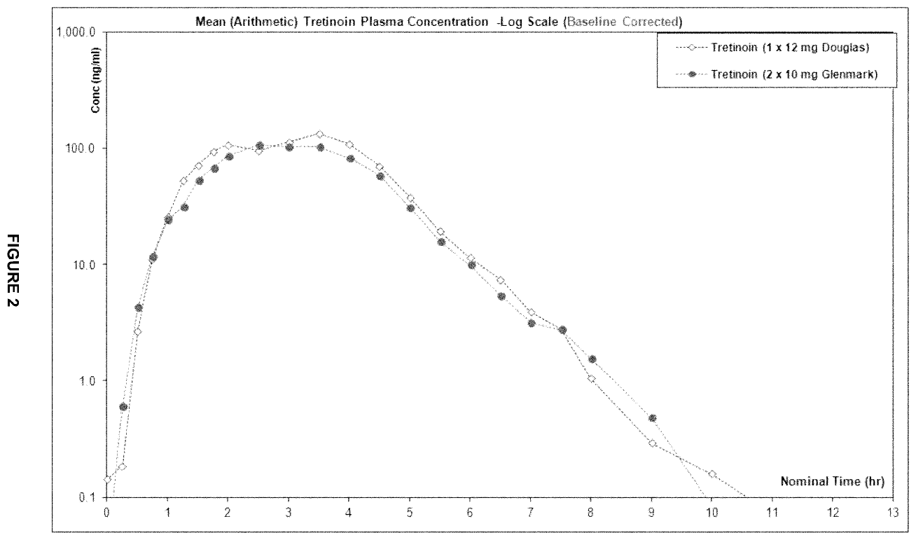

The mean (baseline corrected) tretinoin plasma concentration after single dose of 12 mg (Example 3, 12 mg capsule) tretinoin capsule under fed condition vs a 20 mg dose using the commercially available reference tretinoin soft gel capsule (2×10 mg capsule) in shown in FIG. 2. Roughly they are comparable, but the dose of new formulation is only about half of marketed tretinoin (12 mg vs 20 mg).

The pharmacokinetic parameters for baseline corrected tretinoin (geometric data) are shown in Table 11 and the comparison of the bioavailability in Table 12.

TABLE 11
Pharmacokinetic Parameters for Baseline Corrected
Tretinoin (Geometric data, baseline corrected)

| | | | |
|---|---|---|---|
| AUC_{0-∞} | AUC₀₋ₜ | Cₘₐₓ | Tₘₐₓ |
| (ng · hr/ml) | (ng · hr/ml) | (ng/ml) | (hr) |

Test (1 × 12 mg capsule tretinoin - Formulation Example 3)

| | | | | |
|---|---|---|---|---|
| Geometric | 405.39 | 404.72 | 231.08 | 2.65 |
| Mean | | | | |
| S.D ^{a} | 68.09 | 68.54 | 47.13 | 1.11 |
| Range | 291.80-561.80 | 288.90-561.76 | 159.51-320.90 | 1.00-4.50 |

Reference (2 × 10 mg capsules, commercially available)

| | | | | |
|---|---|---|---|---|
| Geometric | 330.13 | 329.08 | 177.47 | 2.63 |
| Mean | | | | |
| S.D ^{a} | 94.88 | 95.31 | 61.21 | 0.83 |
| Range | 218.42-670.38 | 217.70-670.11 | 103.21-369.75 | 1.50-4.52 |
| P-Values | 0.0166 | 0.0163 | 0.0018 | — |
| Sequence | 0.0323 | 0.0313 | 0.0622 | — |
| Period | 0.5232 | 0.5181 | 0.8513 | — |

^{a} Jackknife Standard Deviation

TABLE 12
Bioavailability analysis of Test (12 mg Tretinoin
capsules) versus Reference (2 × 10 mg Tretinoin
capsules) for baseline corrected tretinoin.

| | Least squares geometric | 90% confidence | Intra |
|---|---|---|---|
| | mean ratio % | interval | subject CV % |
| AUC_{0-∞} | 122.80 | (107.48, 139.40) | 20.31 |
| AUC₀₋ₜ | 122.98 | (107.60, 139.66) | 20.37 |
| Cₘₐₓ | 130.21 | (115.56, 146.48) | 18.49 |

The parameters AUC_{0-∞}, AUC₀₋ₜ and Cₘₐₓ for baseline corrected tretinoin were significantly different between the Test (A) 12 mg Tretinoin capsules, compared with the Reference (B) 2×10 mg Tretinoin capsules.

The 90% confidence intervals for the difference between the Test and the Reference formulation least-squares means for the parameters AUC_{0-∞}, AUC₀₋ₜ and Cₘₐₓ for baseline corrected tretinoin, using log-transformed data were not within 80.00%—125.00%, the accepted limit for demonstrating bioequivalence.

Relative bioavailability of tretinoin was determined by comparing the ratio of the dose adjusted AUC_{0-∞} of Tretinoin 1×12 mg capsule (Test) to that of Tretinoin 2×10 mg capsules (Reference). That is, relative bioavailability is

$${F}_{rel} = \frac{{[ A U {C}_{0 - ∞} ]}_{12 mg}}{{[ A U {C}_{0 - ∞} ]}_{20 mg}} ⨯ \frac{20}{12} ⨯ 100 \%$$

The relative bioavailability was 198.81%.

This study showed the Test capsule, was more bioavailable than the Reference capsule.

### Example 6—Fasting Bioavailability Study

A study was completed in New Zealand to compare the bioavailability of tretinoin for 1×12 mg Tretinoin capsule (Formulation Example 3, 12 mg capsules), relative to that of a Reference formulation, (1×10 mg Tretinoin capsule, Glenmark Pharmaceuticals, USA), in healthy, adult, male subjects under fasting conditions in a single-centre, single-dose, two-treatment, two-period, two-sequence, two-way crossover, randomized study.

Each subject was randomly assigned to one of the treatment sequences shown in Table 13. There was a washout period of at least 2 weeks between treatments. Seventeen 17 subjects received at least 1 dose of study medication and are included in the study population. The 17 male subjects that participated in the study had a mean (range) age of 25 (19-45) years, a mean (range) weight of 76.2 (53.9-90.3) kg, a mean (range) height of 1.80 (1.68-1.90) m and a mean (range) BMI of 23.6 (18.1-28.1) kg/m². There were 15 (88.24%) subjects who were White, 1 (5.88%) subject who was Other Pacific Islander and 1 (5.88%) subject who was White/Other Pacific Islander/Asian. Fifteen subjects completed the study in accordance with the Protocol.

TABLE 13
Treatment sequences

| | Period 1 | Period 2 |
|---|---|---|
| Sequence 1 | Test (1 × 12 mg capsule | Refernce (1 × 10 mg |
| | tretinoin - Formulation | capsule, commercially |
| | Example 3) | available, Glenmark) |
| Sequence 2 | Reference (1 × 10 mg | Test (1 × 12 mg capsule |
| | capsule, commercially | tretinoin - Formulation |
| | available, Glenmark) | Example 3) |

Subjects attended the Clinical Site the evening prior to dosing and fasted overnight for at least 10 hours. Subjects were randomly assigned to one of the two study treatments. The treatments were administered after pre-dose clinical assessments were complete and baseline blood samples (−1, −0.5 and 0 hours) were collected. The treatments were administered orally with 240 ml of ambient-temperature water. The capsules were swallowed whole. Subjects remained at the Clinical Site for 12 hours after dosing for the collection of blood samples (8 ml) through venous catheters at: 0.25, 0.5, 0.75, 1, 1.25, 1.5, 1.75, 2, 2.5, 3, 3.5, 4, 4.5, 5, 5.5, 6, 6.5, 7, 7.5, 8, 9, 10, 11 and 12 hours.

During each study period, water was provided ad libitum until 1.0 hour pre-dose and from 1.0 hour post-dose. The only fluid intake allowed during the intervening two-hour time span was 240 ml of ambient temperature water at dosing. Subjects were required to fast for at least 4 hours after the drug administration. Each subject was provided with a standard lunch and dinner at approximately 12.00 μm and 5.00 μm on the study days. Subjects consumed only the standard meals provided at the Clinical Site and no additional food was allowed on the study days whilst subjects were at the Clinical Site.

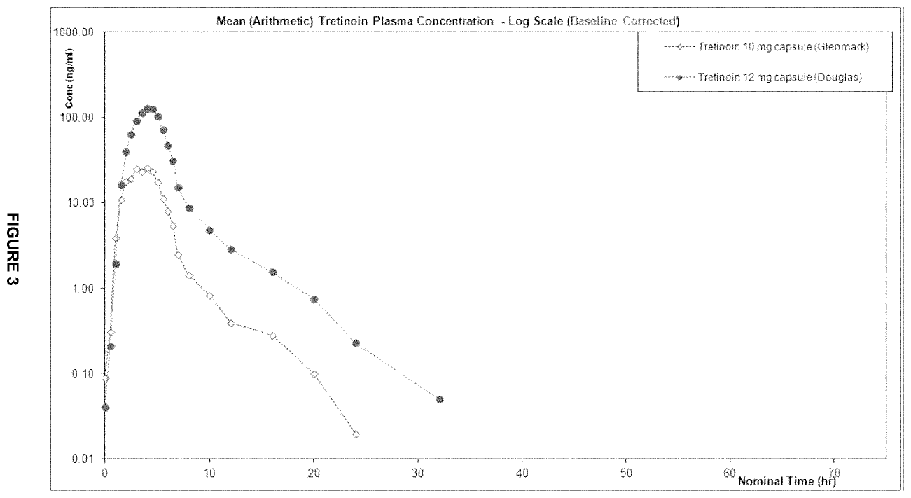

The mean (baseline corrected) tretinoin plasma concentration-time data is shown graphically as plasma profiles in FIG. 3.

The pharmacokinetic parameters for baseline corrected tretinoin (geometric data) are shown in Table 14 and the comparison of the bioavailability in Table 15.

TABLE 14
Pharmacokinetic Parameters for Baseline Corrected
Tretinoin (Geometric data, baseline corrected)

| | | | |
|---|---|---|---|
| AUC_{0-∞} | AUC₀₋ₜ | Cₘₐₓ | Tₘₐₓ |
| (ng · hr/ml) | (ng · hr/ml) | (ng/ml) | (hr) |

Test (1 × 12 mg capsule tretinoin - Test, Formulation Example 3)

| | | | | |
|---|---|---|---|---|
| Geometric | 323.04 | 322.80 | 144.28 | 2.29 |
| Mean | | | | |
| S.D ^{a} | 102.18 | 102.16 | 58.11 | 0.52 |
| Range | 166.10-506.68 | 165.77-506.42 | 62.85-243.27 | 1.75-3.50 |

Reference (1 × 10 mg capsules, Reference)

| | | | | |
|---|---|---|---|---|
| Geometric | 68.49 | 68.22 | 32.30 | 1.74 |
| Mean | | | | |
| S.D ^{a} | 20.46 | 20.43 | 9.20 | 0.50 |
| Range | 46.76-103.04 | 46.67-103.00 | 19.99-52.11 | 1.00-3.02 |
| P-Values | 0.0001 | 0.0001 | 0.0001 | — |
| Sequence | 0.4963 | 0.5024 | 0.3224 | — |
| Period | 0.8288 | 0.8401 | 0.9356 | — |

^{a} Jackknife Standard Deviation

TABLE 15
Bioavailability analysis of Test (12 mg Tretinoin
capsules) versus Reference (1 × 10 mg Tretinoin
capsules) for baseline corrected tretinoin.

| | Least squares geometric | 90% confidence | Intra |
|---|---|---|---|
| | mean ratio % | interval | subject CV % |
| AUC_{0-∞} | 471.64 | (409.36, 544.69) | 22.36 |
| AUC₀₋ₜ | 473.15 | (410.52, 546.54) | 22.40 |
| Cₘₐₓ | 446.63 | (368.77, 541.58) | 30.38 |

The parameters AUC_{0-∞}, AUC₀₋ₜ and Cₘₐₓ for baseline corrected tretinoin were significantly different between the Test, compared with the Reference capsules.

The 90% confidence intervals for the difference between the Test and the Reference capsules least-squares means for the parameters AUC_{0-∞}, AUC₀₋ₜ and Cₘₐₓ for baseline corrected tretinoin, using log-transformed data were not within 80.00%—125.00%, the accepted limit for demonstrating bioequivalence. The 90% confidence intervals for the parameters AUC_{0-∞}, AUC₀₋ₜ and Cₘₐₓ for baseline corrected tretinoin being (409.36,544.69), (410.52,546.54) and (368.77,541.58), respectively.

The relative bioavailability was 393.84%.

Based on these results it can be concluded that the Test capsule is more bioavailable than the Reference capsule.

The blood concentration of tretinoin of the Test capsule is much higher than that of the Reference capsule, and on a similar scale to that for the fed conditions. This suggests the food effect is much less for the Test capsule compared with the Reference capsule.

Comparing Results from Fed and Fasting Bioavailability Studies

Table 16 shows the Cₘₐₓ and AUC_{0-∞} (shown in brackets) relative to the values for the 10 mg Reference product Fed as a percentage (%).

In the Fed trial 20 mg of Reference product was used, so the mean Cₘₐₓ and AUC_{0-∞} measured in the trial were halved to give the “10 mg Reference product Fed”.

The food effect of the formulations as disclosed herein is about 125% (AUC_{0-∞}) and 160% (Cₘₐₓ), compared to about 243% (AUC_{0-∞}) and 274% (Cₘₐₓ) for the Reference product.

TABLE 16
Comparison of Cₘₐₓ and AUC_{0-∞} for 10 mg Reference,
12 mg Test and 5 mg Calculated, fed and fasted.

| | | | | | 5 mg example | |
|---|---|---|---|---|---|---|
| | 10 mg | | 12 mg example | | formulation | |
| | Reference | | formulation | | (calculated) | |
| | Cₘₐₓ | | Cₘₐₓ | | Cₘₐₓ | |
| | (AUC_{0-∞}) | | (AUC_{0-∞}) | | (AUC_{0-∞}) | |
| | relative to 10 mg | | relative to 10 mg | | relative to 10 mg | |
| | Reference product | | Reference product | | Reference product | |
| | Fed % | | Fed % | | Fed % | |
| Fasted | 36 | (41) | 163 | (196) | 68 | (82) |
| Fed | 100 | (100) | 260 | (246) | 109 | (102) |
| Fed/Fasted % | 274 | (243) | 160 | (125) | 160 | (125) |
| Fed/Fasted | 2.74 | (2.43) | 1.60 | (1.25) | 1.60 | (1.25) |

ratio

Table 17 summaries the pharmacokinetics (PK) values (ranges and means) for example formulations according to this disclosure. The values for 5 mg are calculated based on the measured values for the 12 mg example.

TABLE 17
Summaries the PK values
Geometric Data, Baseline Corrected Tretinoin

| | |
|---|---|
| 12 mg | 5 mg (calculated) |

AUC_{0-∞} (ng · hr/ml)

| | | |
|---|---|---|
| Fed | 405.4 | 168.9 |
| range | 291.8-561.8 | 121.6-234.1 |
| Fasted | 323.04 | 134.6 |
| range | 166.1-506.7 | 69.2-211.1 |

Cₘₐₓ (ng/ml)

| | | |
|---|---|---|
| Fed | 231.08 | 96.3 |
| range | 159.5-320.9 | 66.5-133.7 |
| Fasted | 144.28 | 60.1 |
| range | 62.9-243.3 | 26.2-101.4 |

### Example 7—Further Dissolution Testing

The dissolution profile of the of the Test 12 mg Tretinoin capsule (Formulation Example 3, 12 mg capsules), 2×5 mg tretinoin capsules (Formulation Example 1, two 5 mg capsules) and commercially available Glenmark Pharmaceuticals tretinoin 10 mg capsules were measured under alternative dissolution conditions shown in Table 18.

TABLE 18
Dissolution testing conditions

| | |
|---|---|
| Dissolution Medium: | Phosphate buffer containing 0.5% N,N- |
| | dimethyldodecane base amine N-oxide |
| | (LDAO CAS No: 1643-20-5), pH 7.8 |
| Sample Volume | 20 mL |
| Medium Quantity: | 900 mL per vessel |
| Apparatus: | Baskets |
| Temperature | 37.0 ± 0.5° C. |
| Speed: | 100 rpm ± 4 rpm |
| Wavelength measured at | 343 nm |

The 5 mg capsules (Formulation Example 1) were stored for 18 months in 30° C./75% RH and for additional 10 months at controlled room temperature conditions (for example 20-25° C.—see U.S. Pharmacopeia controlled room temperature conditions) prior to testing.

The 12 mg capsules (Formulation Example 3.12 mg capsules) were stored for 36 months in 25° C./60% RH and for additional 16 months at controlled room temperature conditions prior to testing.

The Glenmark Pharmaceuticals tretinoin 10 mg capsules were stored for 1 month at controlled room temperature conditions prior to testing.

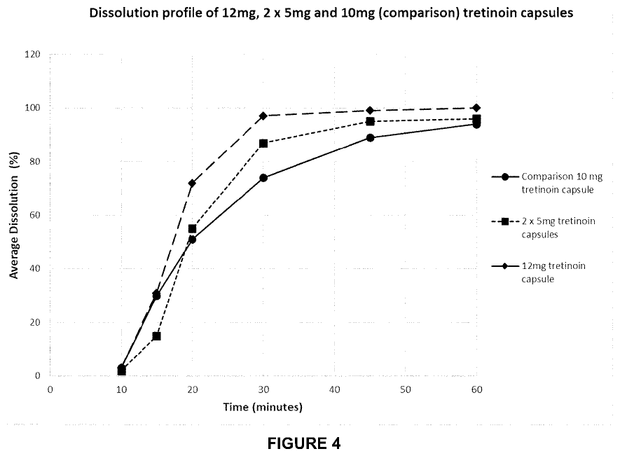

12 samples of each of the test Products were measured separately and the mean dissolution profile calculated. The results are shown in Table 19 and FIG. 4.

TABLE 19
Dissolution results

| | Average % Dissolution | | | | | |
|---|---|---|---|---|---|---|
| | Time (minutes) | | | | | |
| Test product | 10 | 15 | 20 | 30 | 45 | 60 |
| 2 × 5 mg tretinoin capsules | 2 | 15 | 55 | 87 | 95 | 96 |
| (i.e. 10 mg in total), | | | | | | |
| Formulation Example 1 | | | | | | |
| (n = 12) | | | | | | |
| 12 mg tretinoin capsules, | 3 | 31 | 72 | 97 | 99 | 100 |
| Formulation Example 3 | | | | | | |
| (n = 12) | | | | | | |
| Comparative - 10 mg | 3 | 30 | 51 | 74 | 89 | 94 |
| tretinoin capsules -Glenmark | | | | | | |
| Pharmaceuticals | | | | | | |
| (n = 12) | | | | | | |

The dissolution rate of the 2×5 mg capsules and the 12 mg capsules was similar between 15 and 30 minutes (e.g. the steepness of the line), whereas the comparative Example (Glenmark 10 mg capsules) has a slower dissolution between 15 and 30 minutes.

The initial rate of dissolution (i.e the time it takes to start dissolving) may be primarily due to the composition of the capsule shell. Once the shell has released the fill, the rate of dissolution is likely primarily due to the capsule fill formulation.

The start of release and speed of release of the 12 mg tretinoin capsules are different to Example 4 as the dissolution was measured under different conditions. The baskets test apparatus is considered less aggressive than the paddles.

While the invention has been described in detail and with reference to specific examples thereof, it will be apparent to one skilled in the art that various changes and modifications can be made therein without departing from the spirit and scope thereof.

### Numbered Clauses:

1. A pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the active agent has a particle size distribution of D[v,0.5] between about 1 and 10 μm.

2. A pharmaceutical composition in capsule form comprising a capsule shell filled with a capsule fill, the capsule fill comprising:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the active agent has a particle size distribution of D[v,0.5] between about 1 and 10 μm.

3. A pharmaceutical capsule fill composition for a pharmaceutical capsule, the capsule fill comprising:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the active agent has a particle size distribution of D[v,0.5] between about 1 and 10 μm.

4. A pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the capsule is a 12 mg active agent capsule or one or more capsules providing a dose equivalent to a 12 mg active agent capsule and when the capsule is administered to a patient in a fasted state the capsule provides at least one pharmacokinetic parameter selected from the group consisting of:
a mean plasma tretinoin Cₘₐₓ baseline corrected of between about 62 and 243 ng/mL; and
a mean plasma tretinoin AUC_{0-∞} baseline corrected of between about 165 and 507 ng·h/mL.

5. A pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant;
wherein the capsule provides release of at least 80% of the active in vitro at about pH 7.5 to about pH 8 (preferably pH 7.8) within about 30 minutes, preferably at least 85%, preferably at least 90%, preferably at least 95%.

6. A pharmaceutical composition in capsule form wherein the capsule comprises capsule fill, wherein the capsule fill comprises:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant.

7. A unit dosage form for oral administration, the unit dosage form comprising:
a capsule shell,
a capsule fill,
wherein the capsule fill comprises;
   a therapeutically effective amount of an active agent selected from the group consisting of tretinoin, pharmaceutically acceptable salts thereof, and combinations thereof,
   at least one carrier;
   optionally at least one viscosity modifier;
   optionally at least one chelating agent;
   optionally at least one antioxidant;
wherein the active agent has a particle size distribution of D[v,0.5] between about 1 and 10 μm.

8. A pharmaceutical composition comprising:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent;
optionally at least one antioxidant,
wherein the pharmaceutical composition is formulated for oral administration.

9. A method of making a capsule fill for a pharmaceutical composition in capsule form, the method comprising the steps:
adding an active agent selected from the group consisting of tretinoin, pharmaceutically acceptable salts thereof, and combinations thereof to at least one carrier,
wherein the active agent has a particle size distribution of D[v,0.5] between about 1 and 10 μm.

10. A method of making a pharmaceutical composition in capsule form, the method comprising the step of:
encapsulating the capsule fill, as described in any one of clauses 1 to 7, in a capsule shell.

11. The pharmaceutical composition or unit dosage form or method of any one of clauses 1 to 10, wherein the active agent has a particle size distribution of D[v,0.5] between about 1 and 10 μm, or between about 2 and 10 μm, or between about 3 and 10 μm, or between about 3 and 9 μm, or between about 3 and 8 μm, or between about 4 and 8 μm, or between about 5 and 8 μm, or between about 6 and 8 μm, or about 5 μm; or the active agent has a particle size distribution of D[v,0.5] selected from the group consisting of about 1 μm, about 2 μm, about 3 μm, about 4 μm, about 5 μm, about 6 μm, about 7 μm, about 8 μm, about 9 μm, about 10 μm; or
the active agent has a particle size distribution of D[v,0.5] selected from the group consisting of about 5 μm, about 6 μm, about 7 μm, about 8 μm.

12. The pharmaceutical composition or unit dosage form or method of any one of clauses 1 to 11 wherein the active agent has a particle size distribution of D[v,0.1] of not less than about 0.1 μm, or not less than about 0.2 μm, or not less than about 0.3 μm, or not less than about 0.4 μm, or not less than about 0.5 μm, or not less than about 0.6 μm, or not less than about 0.7 μm, or not less than about 0.8 μm, or not less than about 0.9 μm, or not less than about 1 μm, or not less than about 1.1 μm, or not less than about 1.2 μm, or not less than about 1.3 μm, or not less than about 1.4 μm, or not less than about 1.5 μm, or not less than about 1.6 μm, or not less than about 1.7 μm, or not less than about 1.8 μm, or not less than about 1.9 μm, or not less than about 2 μm; or
active agent has a particle size distribution of D[v,0.1] between about 0.1 and 3 μm, or between about 0.2 and 3 μm, or between about 0.3 and 3 μm, or between about 0.4 and 3 μm, or between about 0.5 and 3 μm, or between about 0.6 and 3 μm, or between about 0.7 and 3 μm, or between about 0.8 and 3 μm, or between about 0.9 and 3 μm, or between about 1 and 3 μm; or
the active agent has a particle size distribution of D[v,0.1] between about 0.1 and 2.9 μm, or between about 0.2 and 2.9 μm, or between about 0.3 and 2.9 μm, or between about 0.4 and 2.9 μm, or between about 0.5 and 2.9 μm, or between about 0.6 and 2.9 μm, or between about 0.7 and 2.9 μm, or between about 0.8 and 2.9 μm, or between about 0.9 and 2.9 μm, or between about 1 and 2.9 μm.

13. The pharmaceutical composition or unit dosage form or method of any one of clauses 1 to 12 wherein the active agent has a particle size distribution of D[v,0.9] of not more than about 35 μm, or not more than about 30 μm, or not more than about 29 μm, or not more than about 28 μm, or not more than about 27 μm, or not more than about 26 μm, or not more than about 25 μm, or not more than about 24 μm, or not more than about 23 μm, not more than about 22 μm, or not more than about 21 μm, not more than about 20 μm, or not more than about 19 μm, or not more than about 18 μm; or
the active agent has a particle size distribution of D[v,0.9] between about 8 and 35 μm, or between about 9 and 35 μm, or between about 10 and 35 μm, or between about 10 and 30 μm, or between about 10 and 29 μm, or between about 10 and 28 μm, or between about 10 and 27 μm, or between about 10 and 26 μm, or between about 10 and 25 μm, or between about 10 and 24 μm, or between about 10 and 23 μm, or between about 10 and 22 μm, or between about 10 and 21 μm, or between about 10 and 20 μm, or between about 10 and 19 μm, or between about 11 and 19 μm, or between about 11 and 18 μm, or between about 15 and 18 μm, or between about 11 and 13 μm; or
the active agent has a particle size distribution of D[v,0.9] selected from the group consisting of about 11 μm, about 12 μm, about 13 μm, about 14 μm, about 15 μm, about 16 μm, about 17 μm, about 18 μm.

14. The pharmaceutical composition or unit dosage form or method of any one of clauses 1 to 13 wherein the active agent has a particle size distribution defined by:
D[v,0.1] not less than about 0.5 μm, D[v,0.5] between about 1 and 10 μm, and D[v,0.9] not more than 35 μm; or
D[v,0.1] not less than about 0.5 μm, D[v,0.5] between about 1 and 10 μm, and D[v,0.9] not more than 30 μm; or
D[v,0.1] not less than about 0.5 μm, D[v,0.5] between about 1 and 10 μm, and D[v,0.9] not more than 25 μm; or
D[v,0.1] not less than about 0.5 μm, D[v,0.5] between about 1 and 10 μm, and D[v,0.9] not more than 20 μm; or
D[v,0.1] not less than about 0.5 μm, D[v,0.5] between about 3 and 10 μm, and D[v,0.9] not more than 35 μm; or
D[v,0.1] between about 0.5 and 3 μm, D[v,0.5] between about 3 and 10 μm, and D[v,0.9] between about 10 and 30 μm; or
D[v,0.1] between about 0.5 and 3 μm, D[v,0.5] between about 3 and 10 μm, and D[v,0.9] between about 10 and 20 μm; or
D[v,0.1] between about 0.5 and 3 μm, D[v,0.5] between about 3 and 10 μm, and D[v,0.9] between about 10 and 20 μm; or
D[v,0.1] between about 1 and 3 μm, D[v,0.5] between about 3 and 10 μm, and D[v,0.9] between about 10 and 20 μm.

15. The pharmaceutical composition or unit dosage form or method of any one of clauses 1 to 14 wherein the active agent is tretinoin.

16. The pharmaceutical composition or unit dosage form or method of any one of clauses 1 to 15 wherein the active agent is present in the capsule or unit dosage form in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w, of the capsule or unit dosage form; and/or
the active agent is present in the capsule fill in a range of about 1 to 55% w/w of the capsule fill, or about 2 to 50% w/w of the capsule fill, or about 2 to 40% w/w of the capsule fill, or about 2 to 30% w/w of the capsule fill, or about 2 to 20% w/w of the capsule fill, or about 2 to 15% w/w of the capsule fill, or about 5 to 15% w/w of the capsule fill, or about 5 to 10% w/w of the capsule fill; and/or
the amount of active agent in the capsule or unit dosage form is about 2 to 60 mg, or about 2 to 50 mg, or about 2 to 40 mg, or about 2 to 30 m, or about 2 to 25 mg; or
the amount of active agent in the capsule or unit dosage form is selected from the group consisting of about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 36 mg, about 40 mg, about 45 mg, about 48 mg, about 50 mg and about 60 mg; or
the amount of active agent in the capsule is about 5 mg; or
the amount of active agent in the capsule is about 10 mg; or
the amount of active agent in the capsule is about 12 mg; or
the amount of active agent in the capsule is about 15 mg; or
the amount of active agent in the capsule is about 20 mg.

17. The pharmaceutical composition or unit dosage form or method of any one of clauses 1 to 16 wherein the active agent is substantially suspended in the carrier.

18. The pharmaceutical composition or unit dosage form or method of any one of clauses 1 to 17 wherein the carrier is non-aqueous.

19. The pharmaceutical composition or unit dosage form or method of any one of clauses 1 to 18 wherein the carrier is a liquid.

20. The pharmaceutical composition or unit dosage form or method of any one of clauses 1 to 19 wherein the carrier comprises an oil.

21. The pharmaceutical composition or unit dosage form or method of any one of clauses 1 to 20 wherein the carrier is selected from the group consisting of a mineral oil, a vegetable oil, a fatty acid, a fatty acid ester, a glyceride, a fatty alcohol, a hydrogenated oil, a phospholipid, and combinations thereof; and/or
the carrier comprises a vegetable oil; and/or
the carrier comprises soybean oil; and/or
the carrier is present in the capsule or unit dosage form in a range of about 15 to 72% w/w, or about 18 to 72% w/w, or about 18 to 68% w/w, or about 20 to 68% w/w, or about 20 to 64% w/w, or about 21 to 64% w/w, or about 21 to 60% w/w, of the capsule or unit dosage form; and/or
the carrier is present in the capsule fill in a range of about 50 to 90% w/w of the capsule fill, or about 60 to 90% w/w of the capsule, or about 60 to 85% w/w of the capsule fill, or about 65 to 85% w/w of the capsule fill, or about 65 to 80% w/w of the capsule fill, or about 70 to 80% w/w of the capsule fill, or about 70 to 75% w/w of the capsule fill.

22. The pharmaceutical composition or unit dosage form or method of any one of clauses 1 to 21 wherein the viscosity modifier is selected from the group consisting of hydrogenated vegetable oil, wax, glycerol monostearate, glyceryl behenate, magnesium aluminum silicate, propylene glycol alginate, fatty alcohol and combinations thereof; and/or
the viscosity modifier has a melting point of greater than or equal to 20° C.; and/or
the viscosity modifier has a melting point between about 20 and 85° C.; and/or
the viscosity modifier is a combination of two or more viscosity modifiers that all have a melting point between about 20 and 85° C.; and/or
the viscosity modifier comprises a hydrogenated vegetable oil; and/or
the viscosity modifier comprises hydrogenated vegetable oil Type I and hydrogenated vegetable oil Type II; and/or
the viscosity modifier comprises a wax, for example, the wax is a natural wax, petroleum wax and/or synthetic wax, for example the wax is selected from yellow wax, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, or mixtures thereof; and/or
the viscosity modifier comprises a hydrogenated vegetable oil and a wax.
the viscosity modifier comprises hydrogenated vegetable oil Type I and hydrogenated vegetable oil Type II and a wax; and/or
the viscosity modifier is present in the capsule or unit dose form in a range of about 0.6 to 24% w/w, or about 0.6 to 20% w/w, or about 1.5 to 20% w/w, or about 3 to 20% w/w, or about 5 to 20% w/w, or about 5 to 16% w/w, of the capsule or unit dosage form; and/or
the viscosity modifier is present in the capsule fill in a range of about 2 to 30% w/w, or about 2 to 25% w/w, or about 5 to 25% w/w, or about 10 to 25% w/w, or about 15 to 25% w/w, or about 15 to 20% w/w.

23. The pharmaceutical composition or unit dosage form or method of any one of clauses 1 to 22 wherein the chelating agent is selected from the group consisting of ethylenediamine tetraacetic acid (EDTA), disodium edetate, malic acid, citric acid or a pharmaceutically acceptable salt or hydrate thereof, and combinations thereof; and/or
the chelating agent comprises disodium edetate; and/or
the chelating agent is present in the capsule or unit dosage form in a range of about 0.003 to 8% w/w, or about 0.003 to 4% w/w, or about 0.003 to 2.4% w/w, or about 0.003 to 1.6% w/w, or about 0.003 to 0.8% w/w, or about 0.01 to 0.8% w/w, or about 0.03 to 0.8% w/w, or about 0.03 to 0.6% w/w, of the capsule or unit dosage form; and/or
the chelating agent is present in the capsule fill in a range of about 0.01 to 10% w/w, or about 0.01 to 5% w/w, or about 0.01 to 3% w/w, or about 0.01 to 2% w/w, or about 0.01 to 1% w/w, or about 0.05 to 1% w/w, or about 0.1 to 1% w/w, or about 0.1 to 0.8% w/w, of the capsule fill.

24. The pharmaceutical composition or unit dosage form or method of any one of clauses 1 to 23 wherein the antioxidant is selected from the group consisting of butylated hydroxyanisole (BHA), butylated hydroxytoluene (BHT), propyl gallate, tocopherols (e.g., α-tocopherol (vitamin E)), propionic acid, sodium nitrate, sodium nitrite, citric acid (for example citric acid monohydrate) and combinations thereof; and/or
the antioxidant comprises butylated hydroxyanisole (BHA); and/or
the antioxidant comprises vitamin E; and/or
the antioxidant comprises butylated hydroxyanisole (BHA) and vitamin E; and/or
the antioxidant is present in the capsule or unit dosage form in a range of about 0.0003 to 8%, or about 0.003 to 4% w/w of the capsule fill, or about 0.003 to 2.5% w/w, of the capsule or unit dosage form; and/or
the antioxidant is present in the capsule fill in a range of about 0.001 to 10%, or about 0.01 to 5% w/w of the capsule fill, or about 0.01 to 3% w/w, of the capsule fill.

25. The pharmaceutical composition or unit dosage form or method of any one of clauses 1 to 24 wherein the active agent is present in the capsule or unit dosage form in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form; the at least one carrier is present in a range of about 15 to 72% w/w of the capsule or unit dosage form; the at least one viscosity modifier is present in a range of about 0.6 to 20% w/w of the capsule or unit dosage form; or
the active agent is present in the capsule or unit dosage form in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form; the at least one carrier is present in a range of about 15 to 72% w/w of the capsule or unit dosage form; the at least one viscosity modifier is present in a range of about 0.6 to 20% w/w of the capsule or unit dosage form; the at least one antioxidant is present in a range of about 0.003 to 8% w/w of the capsule or unit dosage form; or
the active agent is present in the capsule or unit dosage form in a range of about 0.3 to 44% w/w, or about 0.6 to 40% w/w, or about 0.6 to 32% w/w, or about 0.6 to 24% w/w, or about 0.6 to 16% w/w, or about 0.6 to 12% w/w, or about 1.5 to 12% w/w, or about 1.5 to 8% w/w of the capsule or unit dosage form; the at least one carrier is present in a range of about 15 to 72% w/w of the capsule or unit dosage form; the at least one viscosity modifier is present in a range of about 0.6 to 20% w/w of the capsule or unit dosage form; the at least one chelating agent is present in a range of about 0.003 to 8% w/w of the capsule or unit dosage form; the at least one antioxidant is present in a range of about 0.003 to 8% w/w of the capsule or unit dosage form; or
the active agent is present in the capsule or unit dosage form in a range of about 0.6 to 24% w/w of the capsule or unit dosage form; the at least one carrier is present in a range of about 15 to 72% w/w of the capsule or unit dosage form; the at least one viscosity modifier is present in a range of about 0.6 to 20% w/w of the capsule or unit dosage form; the at least one chelating agent is present in a range of about 0.003 to 8% w/w of the capsule or unit dosage form; the at least one antioxidant is present in a range of about 0.003 to 8% w/w of the capsule or unit dosage form; or
the active agent is present in the capsule fill in a range of about 1 to 55% w/w, or about 2 to 50% w/w, or about 2 to 40% w/w, or about 2 to 30% w/w, or about 2 to 20% w/w, or about 5 to 20% w/w of the capsule fill; the at least one carrier is present in a range of about 50 to 90% w/w of the capsule fill; the at least one viscosity modifier is present in a range of about 2 to 25% w/w of the capsule fill; or
the active agent is present in the capsule fill in a range of about 1 to 55% w/w, or about 2 to 50% w/w, or about 2 to 40% w/w, or about 2 to 30% w/w, or about 2 to 20% w/w, or about 5 to 20% w/w of the capsule fill; the at least one carrier is present in a range of about 50 to 90% w/w of the capsule fill; the at least one viscosity modifier is present in a range of about 2 to 25% w/w of the capsule fill; the at least one antioxidant is present in a range of about 0.01 to 10% w/w of the capsule fill; or
the active agent is present in the capsule fill in a range of about 1 to 55% w/w, or about 2 to 50% w/w, or about 2 to 40% w/w, or about 2 to 30% w/w, or about 2 to 20% w/w, or about 5 to 20% w/w of the capsule fill; the at least one carrier is present in a range of about 50 to 90% w/w of the capsule fill; the at least one viscosity modifier is present in a range of about 2 to 25% w/w of the capsule fill; the at least one chelating agent is present in a range of about 0.01 to 10% w/w of the capsule fill; the at least one antioxidant is present in a range of about 0.01 to 10% w/w of the capsule fill; or
the active agent is present in the capsule fill in a range of about 2 to 30% w/w of the capsule fill; the at least one carrier is present in a range of about 50 to 90% w/w of the capsule fill; the at least one viscosity modifier is present in a range of about 2 to 25% w/w of the capsule fill; the at least one chelating agent is present in a range of about 0.01 to 10% w/w of the capsule fill; the at least one antioxidant is present in a range of about 0.01 to 10% w/w of the capsule fill.

26. The pharmaceutical composition or unit dosage form or method of any one of clauses 1 to 25 wherein the capsule and/or capsule fill contains less than about 5% w/w isotretinoin, or less than about 2% w/w isotretinoin, or less than about 1% w/w isotretinoin, or less than about 0.5% w/w isotretinoin, or less than about 0.2% w/w isotretinoin based on the weight of the active agent; and/or
the capsule and/or capsule fill contains less than about 5% w/w isotretinoin based on the weight of the active agent after being stored at about 25 to 30° C. in an opaque container for about 3 months, or about 6 months, or about 9 months, or about 12 months, or about 18 months, or about 24 months, or about 36 months; and/or
the capsule and/or capsule fill contains less than about 2% w/w isotretinoin based on the weight of the active agent after being stored at about 25 to 30° C. in an opaque container for about 3 months, or about 6 months, or about 9 months, or about 12 months, or about 18 months, or about 24 months, or about 36 months; and/or
the capsule and/or capsule fill contains less than about 1% w/w isotretinoin based on the weight of the active agent after being stored at about 25 to 30° C. in an opaque container for about 3 months, or about 6 months, or about 9 months, or about 12 months, or about 18 months, or about 24 months, or about 36 months; and/or
the capsule and/or capsule fill contains less than about 0.5% w/w isotretinoin based on the weight of the active agent after being stored at about 25 to 30° C. in an opaque container for about 3 months, or about 6 months, or about 9 months, or about 12 months, or about 18 months, or about 24 months, or about 36 months; and/or
the capsule and/or capsule fill contains less than about 0.2% w/w isotretinoin based on the weight of the active agent after being stored at about 25 to 30° C. in an opaque container for about 3 months, or about 6 months, or about 9 months, or about 12 months, or about 18 months, or about 24 months, or about 36 months.

27. The pharmaceutical composition or unit dosage form or method of any one of clauses 1 to 26 wherein the capsule is a soft gelatin capsule; and/or
the capsule shell comprises gelatin, for example the gelatin is present in the capsule in a range of about 20 to 80% w/w of the capsule shell; and/or
the capsule shell comprises a plasticizer, for example the plasticizer is a polyalcohol, for example the polyalcohol is selected from the group glycerol, propylene glycol, polyethylene glycol, sorbitol, sorbitan, mannitol and combinations thereof, for example the plasticizer is selected from the group glycerol, non-crystallizing sorbitol solution and combinations thereof; and/or
the plasticizer is present in the capsule shell in a range of about 1% to 50% w/w, or about 1 to 40% w/w, or about 1 to 30% w/w or about 1 to 25% w/w of the capsule shell.

28. The pharmaceutical composition or unit dosage form or method of any one of clauses 1 to 27 wherein the capsule shell is about 20 to 70% w/w of the capsule.

29. The pharmaceutical composition or unit dosage form or method of any one of clauses 1 to 28 wherein the capsule and/or unit dosage form is a 12 mg active agent capsule or one or more capsules providing a dose equivalent to a 12 mg active agent capsule and when the capsule is administered to a patient in a fed or fasted state, the capsule provides a mean Tₘₐₓ of the active agent selected from the group consisting of at least about 0.5 hours, at least about 1 hour, at least about 1.5 hours, at least about 2 hours, at least about 2.5 hours, between about 0.5 to 6 hours, between about 1 to 5 hours, between about 1 to 3 hours, between about 2 to 3 hours.

30. The pharmaceutical composition or unit dosage form or method of any one of clauses 1 to 29 wherein the capsule and/or unit dosage form is a 12 mg active agent capsule or one or more capsules providing a dose equivalent to a 12 mg active agent capsule and when the capsule is administered to a patient in a fed state, the capsule provides:
a mean plasma tretinoin Cₘₐₓ of at least about 150 ng/mL, or at least about 160 ng/mL, or at least about 180 ng/mL, or at least about 185 ng/mL, or at least about 190 ng/mL, or at least about 200 ng/mL, or at least about 210 ng/mL, or at least about 215 ng/mL, or at least about 220 ng/mL and/or
a mean plasma tretinoin Cₘₐₓ between about 160 and 350 ng/mL, or about 180 and 320 ng/mL, or about 180 and 300 ng/mL, or about 180 and 290 ng/mL, or about 185 and 289 ng/mL, or about 190 and 290 ng/mL, or about 190 and 280 ng/mL, or about 190 and 270 ng/mL, or about 200 and 270 ng/mL, or about 200 and 260 ng/mL, or about 200 and 250 ng/mL, or about 200 and 240 ng/mL, or about 210 and 240 ng/mL, or about 220 and 240 ng/mL.

31. The pharmaceutical composition or unit dosage form or method of any one of clauses 1 to 30 wherein the capsule and/or unit dosage form is a 12 mg active agent capsule or one or more capsules providing a dose equivalent to a 12 mg active agent capsule and when the capsule is administered to a patient in a fed state, the capsule provides:
a mean plasma tretinoin AUC_{0-∞} baseline corrected of at least about 300 ng·h/mL, or at least about 320 ng·h/mL, or at least about 330 ng·h/mL, or at least about 340 ng·h/mL, or at least about 350 ng·h/mL, or at least about 360 ng·h/mL, or at least about 370 ng·h/mL, or at least about 380 ng·h/mL, or at least about 390 ng·h/mL, or at least about 400 ng·h/mL; and/or
a mean plasma tretinoin AUC_{0-∞} baseline corrected of between about 290 and 562 ng·h/mL, or between about 300 and 550 ng·h/mL, or between about 310 and 530 ng·h/mL, or between about 320 and 520 ng·h/mL, or between about 320 and 510 ng·h/mL, or between about 324 and 506 ng·h/mL.

32. The pharmaceutical composition or unit dosage form or method of any one of clauses 1 to 31 wherein the capsule and/or unit dosage form is a 12 mg active agent capsule or one or more capsules providing a dose equivalent to a 12 mg active agent capsule and when the capsule is administered to a patient in a fed state the capsule provides at least one pharmacokinetic parameter selected from the group consisting of:
a mean plasma tretinoin Cₘₐₓ baseline corrected of between about 158 and 321 ng/mL; and
a mean plasma tretinoin AUC_{0-∞} baseline corrected of between about 290 and 562 ng·h/mL.

33. The pharmaceutical composition or unit dosage form or method of any one of clauses 1 to 32 wherein the capsule and/or unit dosage form is a 12 mg active agent capsule or one or more capsules providing a dose equivalent to a 12 mg active agent capsule and when the capsule is administered to a patient in a fed state the capsule provides at least one pharmacokinetic parameter selected from the group consisting of:
a mean plasma tretinoin Cₘₐₓ baseline corrected of between about 180 and 300 ng/mL; and
a mean plasma tretinoin AUC_{0-∞} baseline corrected of between about 320 and 520 ng·h/mL.

34. The pharmaceutical composition or unit dosage form or method of any one of clauses 1 to 33 wherein the capsule and/or unit dosage form is a 12 mg active agent capsule or one or more capsules providing a dose equivalent to a 12 mg active agent capsule and when the capsule is administered to a patient in a fed state the capsule provides at least one pharmacokinetic parameter selected from the group consisting of:
a mean plasma tretinoin Cₘₐₓ baseline corrected of between about 184 and 289 ng/mL; and
a mean plasma tretinoin AUC_{0-∞} baseline corrected of between about 324 and 506 ng·h/mL.

35. The pharmaceutical composition or unit dosage form or method of any one of clauses 1 to 34 wherein the capsule and/or unit dosage form is a 12 mg active agent capsule, or one or more capsules providing a dose equivalent to a 12 mg active agent capsule, and when the capsule is administered to a patient in a fasted state the capsule provides:
a mean plasma tretinoin Cₘₐₓ of at least about 40 ng/mL, or at least about 50 ng/mL, or at least about 60 ng/mL, or at least about 70 ng/mL, or at least about 80 ng/mL, or at least about 90 ng/mL, or at least about 100 ng/mL, or at least about 110 ng/mL, or at least about 120 ng/mL, or at least about 130 ng/mL, or at least about 135 ng/mL, or at least about 140 ng/mL; and/or
a mean plasma tretinoin Cₘₐₓ between about 60 and 250 ng/mL, or between about 62 and 244 ng/mL, or between about 70 and 240 ng/mL, or between about 80 and 230 ng/mL, or between about 90 and 220 ng/mL, or between about 100 and 210 ng/mL, or between about 100 and 200 ng/mL, or between about 110 and 190 ng/mL, or between about 115 and 180 ng/mL, or between about 120 and 170 ng/mL, or between about 130 and 160 ng/mL, or between about 140 and 150 ng/mL.

36. The pharmaceutical composition or unit dosage form or method of any one of clauses 1 to 35 wherein the capsule and/or unit dosage form is a 12 mg active agent capsule, or one or more capsules providing a dose equivalent to a 12 mg active agent capsule, and when the capsule is administered to a patient in a fasted state the capsule provides:
a mean plasma tretinoin AUC_{0-∞} baseline corrected of at least about 100 ng·h/mL, or at least about 120 ng·h/mL, or at least about 140 ng·h/mL, or at least about 160 ng·h/mL, or at least about 180 ng·h/mL, or at least about 200 ng·h/mL, or at least about 220 ng·h/mL, or at least about 240 ng·h/mL, or at least about 258 ng·h/mL, or at least about 260 ng·h/mL, or at least about 280 ng·h/mL, or at least about 300 ng·h/mL, or at least about 310 ng·h/mL, or at least about 315 ng·h/mL, or at least about 320 ng·h/mL; and/or
a mean plasma tretinoin AUC_{0-∞} baseline corrected of between about 100 and 500 ng·h/mL, or between about 150 and 450 ng·h/mL, or between about 200 and 420 ng·h/mL, or between about 240 and 420 ng·h/mL, or between about 258 and 404 ng·h/mL, or between about 260 and 390 ng·h/mL, or between about 280 and 360 ng·h/mL.

37. The pharmaceutical composition or unit dosage form or method of any one of clauses 1 to 36 wherein the capsule and/or unit dosage form is a 12 mg active agent capsule or one or more capsules providing a dose equivalent to a 12 mg active agent capsule and when the capsule is administered to a patient in a fasted state the capsule provides at least one pharmacokinetic parameter selected from the group consisting of:
a mean plasma tretinoin Cₘₐₓ baseline corrected of between about 62 and 243 ng/mL; and
a mean plasma tretinoin AUC_{0-∞} baseline corrected of between about 165 and 507 ng·h/mL.

38. The pharmaceutical composition or unit dosage form or method of any one of clauses 1 to 37 wherein the capsule and/or unit dosage form is a 12 mg active agent capsule or one or more capsules providing a dose equivalent to a 12 mg active agent capsule and when the capsule is administered to a patient in a fasted state the capsule provides at least one pharmacokinetic parameter selected from the group consisting of:
a mean plasma tretinoin Cₘₐₓ baseline corrected of between about 100 and 200 ng/mL; and
a mean plasma tretinoin AUC_{0-∞} baseline corrected of between about 240 and 420 ng·h/mL.

39. The pharmaceutical composition or unit dosage form or method of any one of clauses 1 to 38 wherein the capsule and/or unit dosage form is a 12 mg active agent capsule or one or more capsules providing a dose equivalent to a 12 mg active agent capsule and when the capsule is administered to a patient in a fasted state the capsule provides at least one pharmacokinetic parameter selected from the group consisting of:
a mean plasma tretinoin Cₘₐₓ baseline corrected of between about 115 and 180 ng/mL; and
a mean plasma tretinoin AUC_{0-∞} baseline corrected of between about 258 and 404 ng·h/mL.

40. The pharmaceutical composition or unit dosage form or method of any one of clauses 1 to 28 wherein the capsule and/or unit dosage form is a 5 mg active agent capsule or one or more capsules providing a dose equivalent to a 5 mg active agent capsule and when the capsule is administered to a patient in a fed state the capsule provides:
a mean plasma tretinoin Cₘₐₓ of at least about 50 ng/mL, or at least about 60 ng/mL, or at least about 70 ng/mL, or at least about 77 ng/mL, or at least about 80 ng/mL, or at least about 85 ng/mL, or at least about 90 ng/mL; and/or
a mean plasma tretinoin Cₘₐₓ between about 60 and 150 ng/mL, or between about 70 and 140 ng/mL, or between about 75 and 130 ng/mL, or between about 77 and 120 ng/mL, or between about 80 and 110 ng/mL, or between about 90 and 100 ng/mL.

41. The pharmaceutical composition or unit dosage form or method of any one of clauses 1 to 28 or 40 wherein the capsule and/or unit dosage form is a 5 mg active agent capsule or one or more capsules providing a dose equivalent to a 5 mg active agent capsule and when the capsule is administered to a patient in a fed state the capsule provides:
a mean plasma tretinoin AUC_{0-∞} baseline corrected of at least about 100 ng·h/mL, or at least about 110 ng·h/mL, at least about 115 ng·h/mL, at least about 120 ng·h/mL, at least about 125 ng·h/mL, at least about 130 ng·h/mL, at least about 135 ng·h/mL, at least about 140 ng·h/mL, at least about 145 ng·h/mL, at least about 150 ng·h/mL, at least about 165 ng·h/mL; and/or
a mean plasma tretinoin AUC_{0-∞} baseline corrected of between about 100 and 250 ng·h/mL, or between about 110 and 245 ng·h/mL, or between about 120 and 240 ng·h/mL, or between about 130 and 220 ng·h/mL, or between about 135 and 211 ng·h/mL, or between about 130 and 210 ng·h/mL, or between about 150 and 200 ng·h/mL.

42. The pharmaceutical composition or unit dosage form or method of any one of clauses 1 to 28 or 40 to 41 wherein the capsule and/or unit dosage form is a 5 mg active agent capsule or is one or more capsules providing a dose equivalent to a 5 mg active agent capsule and when the capsule is administered to a patient in a fed state the capsule provides at least one pharmacokinetic parameter selected from the group consisting of:
a mean plasma tretinoin Cₘₐₓ baseline corrected of between about 65 and 135 ng/mL; and
a mean plasma tretinoin AUC_{0-∞} baseline corrected of between about 120 and 235 ng·h/mL.

43. The pharmaceutical composition or unit dosage form or method of any one of clauses 1 to 28 or 40 to 42 wherein the capsule and/or unit dosage form is a 5 mg active agent capsule or is one or more capsules providing a dose equivalent to a 5 mg active agent capsule and when the capsule is administered to a patient in a fed state the capsule provides at least one pharmacokinetic parameter selected from the group consisting of:
a mean plasma tretinoin Cₘₐₓ baseline corrected of between about 70 and 150 ng/mL; and
a mean plasma tretinoin AUC_{0-∞} baseline corrected of between about 130 and 220 ng·h/mL.

44. The pharmaceutical composition or unit dosage form or method of any one of clauses 1 to 28 or 40 to 43 wherein the capsule and/or unit dosage form is a 5 mg active agent capsule or is one or more capsules providing a dose equivalent to a 5 mg active agent capsule and when the capsule is administered to a patient in a fed state the capsule provides at least one pharmacokinetic parameter selected from the group consisting of:
a mean plasma tretinoin Cₘₐₓ baseline corrected of between about 76 and 120 ng/mL; and
a mean plasma tretinoin AUC_{0-∞} baseline corrected of between about 135 and 211 ng·h/mL.

45. The pharmaceutical composition or unit dosage form or method of any one of clauses 1 to 28 or 40 to 44 wherein the capsule and/or unit dosage form is a 5 mg active agent capsule or one or more capsules providing a dose equivalent to a 5 mg active agent capsule and when the capsule is administered to a patient in a fasted state the capsule provides:
a mean plasma tretinoin Cₘₐₓ of at least about 20 ng/mL, or at least about 30 ng/mL, or at least about 35 ng/mL, or at least about 40 ng/mL, or at least about 45 ng/mL, or at least about 50 ng/mL, or at least about 55 ng/mL; and/or
a mean plasma tretinoin Cₘₐₓ between about 20 and 110 ng/mL, or between about 25 and 102 ng/mL, or between about 26 and 101 ng/mL, or between about 40 and 90 ng/mL, or between about 48 and 75 ng/mL.

46. The pharmaceutical composition or unit dosage form or method of any one of clauses 1 to 28 or 40 to 45 wherein the capsule and/or unit dosage form is a 5 mg active agent capsule or one or more capsules providing a dose equivalent to a 5 mg active agent capsule and when the capsule is administered to a patient in a fasted state the capsule provides:
a mean plasma tretinoin AUC_{0-∞} baseline corrected of at least about 40 ng·h/mL, at least about 50 ng·h/mL, at least about 60 ng·h/mL, at least about 70 ng·h/mL, at least about 80 ng·h/mL, at least about 90 ng·h/mL, at least about 100 ng·h/mL, at least about 110 ng·h/mL, at least about 120 ng·h/mL, at least about 130 ng·h/mL and/or
a mean plasma tretinoin AUC_{0-∞} baseline corrected of between about 50 and 250 ng·h/mL, or between about 60 and 220 ng·h/mL, or between about 65 and 215 ng·h/mL, or between about 68 and 212 ng·h/mL, or between about 70 and 200 ng·h/mL, or between about 80 and 180 ng·h/mL, or between about 90 and 180 ng·h/mL, or between about 100 and 170 ng·h/mL, between about 108 and 169 ng·h/mL, or between about 110 and 150 ng·h/mL.

47. The pharmaceutical composition or unit dosage form or method of any one of clauses 1 to 28 or 40 to 46 wherein the capsule and/or unit dosage form is a 5 mg active agent capsule or is one or more capsules providing a dose equivalent to a 5 mg active agent capsule and when the capsule is administered to a patient in a fasted state the capsule provides at least one pharmacokinetic parameter selected from the group consisting of
a mean plasma tretinoin Cₘₐₓ baseline corrected of between about 25 and 102 ng/mL; and
a mean plasma tretinoin AUC_{0-∞} baseline corrected of between about 68 and 212 ng·h/mL.

48. The pharmaceutical composition or unit dosage form or method of any one of clauses 1 to 28 or 40 to 47 wherein said capsule and/or unit dosage form is a 5 mg active agent capsule or is one or more capsules providing a dose equivalent to a 5 mg active agent capsule and when said capsule is administered to a patient in a fasted state said capsule provides at least one pharmacokinetic parameter selected from the group consisting of
a mean plasma tretinoin Cₘₐₓ baseline corrected of between about 40 and 90 ng/mL; and
a mean plasma tretinoin AUC_{0-∞} baseline corrected of between about 90 and 180 ng·h/mL.

49. The pharmaceutical composition or unit dosage form or method of any one of clauses 1 to 28 or 40 to 48 wherein said capsule and/or unit dosage form is a 5 mg active agent capsule or is one or more capsules providing a dose equivalent to a 5 mg active agent capsule and when said capsule is administered to a patient in a fasted state said capsule provides at least one pharmacokinetic parameter selected from the group consisting of
a mean plasma tretinoin Cₘₐₓ baseline corrected of between about 48 and 75 ng/mL; and
a mean plasma tretinoin AUC_{0-∞} baseline corrected of between about 108 and 169 ng·h/mL.

50. The pharmaceutical composition or unit dosage form or method of any one of clauses 1 to 49 wherein the capsule and/or unit dosage form provides release of at least about 25% of the active in vitro within about 10 minutes, or at least about 30%, or at least about 35%, or at least about 40%; and/or
the capsule and/or unit dosage form provides release of at least about 65% of the active in vitro within about 15 minutes, or at least about 70%, or at least about 75%, or at least about 80%; and/or
the capsule and/or unit dosage form provides release of at least about 75% of the active in vitro within about 20 minutes, or at least about 80%, or at least about 85%, or at least about 90%; and/or
the capsule and/or unit dosage form provides release of at least about 80% of the active in vitro within about 30 minutes, or at least about 85%, or at least about 90%, or at least about 95%; and/or
the capsule and/or unit dosage form provides release of at least about 98% of the active in vitro within about 45 minutes, or at least about 99%.

51. The pharmaceutical composition or unit dosage form or method of any one of clauses 1 to 50 wherein the capsule and/or unit dosage form provides release of about 30 to 80%, or about 35 to 60%, or about 38 to 60% of the active in vitro at about 10 minutes; and/or
the capsule and/or unit dosage form provides release of about 60 to 100%, or about 65 to 100%, or about 66 to 100%, of the active in vitro at about 15 minutes; and/or
the capsule and/or unit dosage form provides release of about 70 to 100%, or about 75 to 100%, or about 76 to 100%, of the active in vitro at about 20 minutes; and/or
the capsule and/or unit dosage form provides release of about 70 to 100%, or about 75 to 100%, or about 79 to 100%, of the active in vitro at about 30 minutes; and/or
the capsule and/or unit dosage form provides release of about 75 to 100%, or about 80 to 100%, of the active in vitro at about 45 minutes.

52. The pharmaceutical composition or unit dosage form or method of any one of clauses 1 to 51 wherein the capsule and/or unit dosage form provides release of the active agent when measured in vitro that substantially corresponds to the following dissolution pattern:
(a) between about 30% and 80% of the total active agent is released after about 10 minutes of measurement in the apparatus;
(b) not less than about 60% of the total active agent is released after 20 minutes of measurement in the apparatus; and
(c) not less than 80% of the total active agent is released after about 30 minutes of measurement in the apparatus.

53. The pharmaceutical composition or unit dosage form or method of any one of clauses 1 to 52 wherein the capsule and/or unit dosage form provides release of the active agent when measured in vitro that substantially corresponds to the following dissolution pattern:
(a) between about 38 and 60% of the total active agent is released after about 10 minutes of measurement in the apparatus;
(b) between about 75 and 100% of the total active agent is released after 20 minutes of measurement in the apparatus; and
(c) not less than 79% of the total active agent is released after about 30 minutes of measurement in the apparatus.

54. The pharmaceutical composition or unit dosage form or method of any one of clauses 1 to 53 wherein the capsule and/or unit dosage form provides release of the active agent when measured in vitro that substantially corresponds to the following dissolution pattern:
(a) between about 38 and 60% of the total active agent is released after about 10 minutes of measurement in the apparatus;
(b) between about 65 and 100% of the total active agent is released after 15 minutes of measurement in the apparatus;
(c) not less than 75% of the total active agent is released after about 20 minutes of measurement in the apparatus.

55. The pharmaceutical composition or unit dosage form or method of any one of clauses 1 to 39 or 50 to 54 wherein the capsule and/or unit dosage form is a 12 mg active agent capsule,
the capsule provides release of at least about 25% of the active in vitro within about 10 minutes, or at least about 30%, or at least about 35%, or at least about 40%; and/or
the capsule provides release of at least about 65% of the active in vitro within about 15 minutes, or at least about 70%, or at least about 75%, or at least about 80%; and/or
the capsule provides release of at least about 75% of the active in vitro within about 20 minutes, or at least about 80%, or at least about 85%, or at least about 90%; and/or
the capsule provides release of at least about 80% of the active in vitro within about 30 minutes, or at least about 85%, or at least about 90%, or at least about 95%; and/or
the capsule provides release of at least about 98% of the active in vitro within about 45 minutes, or at least 99%; and/or
the capsule provides release of about 30 to 80%, or about 35 to 60%, or about 38 to 60% of the active in vitro at about 10 minutes; and/or
the capsule provides release of about 60 to 100%, or about 65 to 100%, or about 66 to 100%, of the active in vitro at about 15 minutes; and/or
the capsule provides release of about 70 to 100%, or about 75 to 100%, or about 76 to 100%, of the active in vitro at about 20 minutes; and/or
the capsule provides release of about 70 to 100%, or about 75 to 100%, or about 79 to 100%, of the active in vitro at about 30 minutes; and/or
the capsule provides release of about 75 to 100%, or about 80 to 100%, of the active in vitro at about 45 minutes.

56. The pharmaceutical composition or unit dosage form or method of any one of clauses 1 to 39 or 50 to 55 wherein the capsule and/or unit dosage form is a 12 mg active agent capsule, the capsule provides release of the active agent when measured in vitro that substantially corresponds to the following dissolution pattern:
(a) between about 30% and 80% of the total active agent is released after about 10 minutes of measurement in the apparatus;
(b) not less than about 60% of the total active agent is released after 20 minutes of measurement in the apparatus; and
(c) not less than 80% of the total active agent is released after about 30 minutes of measurement in the apparatus.

57. The pharmaceutical composition or unit dosage form or method of any one of clauses 1 to 39 or 50 to 56 wherein the capsule and/or unit dosage form is a 12 mg active agent capsule, the capsule provides release of the active agent when measured in vitro that substantially corresponds to the following dissolution pattern:
(a) between about 38 and 60% of the total active agent is released after about 10 minutes of measurement in the apparatus;
(b) between about 75 and 100% of the total active agent is released after 20 minutes of measurement in the apparatus; and
(c) not less than 79% of the total active agent is released after about 30 minutes of measurement in the apparatus.

58. The pharmaceutical composition or unit dosage form or method of any one of clauses 1 to 39 or 50 to 57 wherein the capsule and/or unit dosage form is a 12 mg active agent capsule, the capsule provides release of the active agent when measured in vitro that substantially corresponds to the following dissolution pattern:
(a) between about 38 and 60% of the total active agent is released after about 10 minutes of measurement in the apparatus;
(b) between about 65 and 100% of the total active agent is released after 15 minutes of measurement in the
(c) not less than 75% of the total active agent is released after about 20 minutes of measurement in the apparatus.

59. The pharmaceutical composition or unit dosage form or method of any one of clauses 1 to 58 wherein the capsule and/or unit dosage form provides release of the active agent when measured using USP (US Pharmacopoeia) 43-NF38 apparatus II (paddles) according to chapter <711>, at speed 100 rpm±4 rpm, in 900 mL per vessel of dissolution medium 0.05 M phosphate buffer pH 7.8 with 0.5% lauryldimethylamine oxide (LDAO), at 37.0° C.±0.5° C., with spiral capsule sinker,
that substantially corresponds to about 30 to 80%, or about 35 to 60%, or about 38 to 60% of the active in vitro at about 10 minutes from start of measurement in the apparatus; and/or
that substantially corresponds to about 60 to 100%, or about 65 to 100%, or about 66 to 100%, of the active at about 15 minutes from start of measurement in the apparatus; and/or
that substantially corresponds to about 70 to 100%, or about 75 to 100%, or about 76 to 100%, of the total active at about 20 minutes of from start of measurement in the apparatus; and/or
that substantially corresponds to about 70 to 100%, or about 75 to 100%, or about 79 to 100%, of the total active at about 30 minutes of from start of measurement in the apparatus; and/or
that substantially corresponds to about 75 to 100%, or about 80 to 100%, of the total active at about 45 minutes of from start of measurement in the apparatus.

60. The pharmaceutical composition or unit dosage form or method of any one of clauses 1 to 59 wherein the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer pH 7.8 with 0.5% lauryldimethylamine oxide (LDAO), at 37.0° C. 0.5° C., with spiral capsule sinker, that substantially corresponds to the following dissolution pattern:
(a) between about 30% and 80% of the total active agent is released after about 10 minutes of measurement in the apparatus;
(b) not less than about 60% of the total active agent is released after 20 minutes of measurement in the apparatus; and
(c) not less than 80% of the total active agent is released after about 30 minutes of measurement in the apparatus.

61. The pharmaceutical composition or unit dosage form or method of any one of clauses 1 to 60 wherein the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer pH 7.8 with 0.5% lauryldimethylamine oxide (LDAO), at 37.0° C. 0.5° C., with spiral capsule sinker, that substantially corresponds to the following dissolution pattern:
(a) between about 38 and 60% of the total active agent is released after about 10 minutes of measurement in the apparatus;
(b) between about 75 and 100% of the total active agent is released after 20 minutes of measurement in the apparatus; and
(c) not less than 79% of the total active agent is released after about 30 minutes of measurement in the apparatus.

62. The pharmaceutical composition or unit dosage form or method of any one of clauses 1 to 61 wherein the capsule and/or unit dosage form provides release of the active agent when measured in vitro using USP (US Pharmacopoeia) 43-NF38 apparatus II (paddles) according to chapter <711>, at speed 100 rpm±4 rpm, in 900 mL per vessel of dissolution medium 0.05 M phosphate buffer pH 7.8 with 0.5% lauryldimethylamine oxide (LDAO), at 37.0° C. 0.5° C., with spiral capsule sinker, that substantially corresponds to the following dissolution pattern:
(a) between about 38 and 60% of the total active agent is released after about 10 minutes of measurement in the apparatus;
(b) between about 65 and 100% of the total active agent is released after 15 minutes of measurement in the
(c) not less than 75% of the total active agent is released after about 20 minutes of measurement in the apparatus.

63. The pharmaceutical composition or unit dosage form or method of any one of clauses 1 to 62 wherein the pharmaceutical composition is formulated for once daily administration or twice-daily administration to a patient.

64. The pharmaceutical composition or unit dosage form or method of any one of clauses 1 to 62 wherein the pharmaceutical composition is formulated for oral administration.

65. The method of any one of clauses 9 to 64 wherein the active agent is reduced in particle size to the particle size distribution after being added to the at least one carrier; and/or the active agent is milled to the particle size distribution after being added to the at least one carrier; and/or
the active agent is bead milled to the particle size distribution after being added to the at least one carrier.

66. The method of any one of clauses 9 to 64 wherein the active agent is reduced in particle size to the particle size distribution prior to being added to the at least one carrier; and/or
the active agent is milled to the particle size distribution prior to being added to the at least one carrier; and/or
the active agent is dry milled to the particle size distribution prior to being added to the at least one carrier; and/or
the active agent is jet milled to the particle size distribution prior to being added to the at least one carrier.

67. The method of any one of clauses 9 to 66 wherein the active agent is added to at least one carrier and at least one viscosity modifier; and/or
the at least one carrier and the at least one viscosity modifier are combined prior to the addition of the active agent; and/or
the at least one carrier and the at least one viscosity modifier are combined and heated to about 50 to 80° C., or about 60 to 80° C., or about 65 to 75° C.; and/or
the antioxidant is added to the carrier at substantially the same time or prior to the active agent; and/or
the combined carrier and viscosity modifier are cooled to about room temperature prior to addition of the tretinoin.

1. A pharmaceutical composition in a capsule form comprising a capsule fill, wherein the capsule fill comprises:
a therapeutically effective amount of an active agent selected from the group consisting of tretinoin, pharmaceutically acceptable salts thereof, and combinations thereof;
at least one carrier;
at least one viscosity modifier;
optionally at least one chelating agent; and
optionally at least one antioxidant;
wherein the active agent has a particle size distribution of D[v,0.5] between about 1 and 10 μm, and
wherein the at least one viscosity modifier is present in the capsule fill in a range of about 10 to 25% w/w.

2. (canceled)

3. The pharmaceutical composition of claim 1 wherein the active agent has a particle size distribution of D[v,0.1] of not less than about 0.1 m.

4. The pharmaceutical composition of claim 1 wherein the active agent has a particle size distribution of D[v,0.9] of not more than about 25 m.

5. The pharmaceutical composition of claim 1 wherein the active agent is present in the capsule fill in a range of about 5 to 15% w/w of the capsule fill.

6. (canceled)

7. The pharmaceutical composition of claim 1 wherein the carrier is non-aqueous.

8. The pharmaceutical composition of claim 1 wherein the carrier comprises an oil.

9. The pharmaceutical composition of claim 1 wherein the carrier is selected from the group consisting of a mineral oil, a vegetable oil, a fatty acid, a fatty acid ester, a glyceride, a fatty alcohol, a hydrogenated oil, a phospholipid, and combinations thereof; and/or
the carrier comprises a vegetable oil; and/or
the carrier comprises soybean oil.

10. The pharmaceutical composition of claim 1 wherein the carrier is present in the capsule fill in a range of about 50 to 90% w/w of the capsule fill.

11. The pharmaceutical composition of claim 1 wherein the viscosity modifier is selected from the group consisting of hydrogenated vegetable oil, wax, glycerol monostearate, glyceryl behenate, magnesium aluminum silicate, propylene glycol alginate, fatty alcohol and combinations thereof; and/or
the viscosity modifier has a melting point of greater than or equal to 20° C.; and/or
the viscosity modifier comprises a hydrogenated vegetable oil; and/or
the viscosity modifier comprises hydrogenated vegetable oil Type I and hydrogenated vegetable oil Type II; and/or
the viscosity modifier comprises a wax, for example, the wax is a natural wax, petroleum wax and/or synthetic wax, for example the wax is selected from yellow wax, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, or mixtures thereof; and/or
the viscosity modifier comprises a hydrogenated vegetable oil and a wax; and/or
the viscosity modifier comprises hydrogenated vegetable oil Type I and hydrogenated vegetable oil Type II and a wax.

12. The pharmaceutical composition of claim 1 wherein the chelating agent is selected from the group consisting of ethylenediamine tetraacetic acid (EDTA), disodium edetate, malic acid, citric acid or a pharmaceutically acceptable salt or hydrate thereof, and combinations thereof; and/or
the chelating agent is present in the capsule fill in a range of about 0.01 to 10% w/w of the capsule fill.

13. The pharmaceutical composition of claim 1 wherein the antioxidant is selected from the group consisting of butylated hydroxyanisole (BHA), butylated hydroxytoluene (BHT), propyl gallate, tocopherols (e.g., α-tocopherol (vitamin E)), propionic acid, sodium nitrate, sodium nitrite, citric acid (for example citric acid monohydrate) and combinations thereof; and/or
the antioxidant is present in the capsule fill in a range of about 0.001 to 10% w/w of the capsule fill.

14. The pharmaceutical composition of claim 1 wherein the capsule and/or capsule fill contains less than about 0.2% w/w isotretinoin based on the weight of the active agent after being stored at about 25 to 30° C. in an opaque container for about 3 months, or about 6 months, or about 9 months, or about 12 months, or about 18 months, or about 24 months, or about 36 months.

15. The pharmaceutical composition of claim 1 wherein the capsule is a soft gelatin capsule.

16. (canceled)

17. The pharmaceutical composition of claim 1 wherein the capsule is a 12 mg active agent capsule or one or more capsules providing a dose equivalent to a 12 mg active agent capsule and when the capsule is administered to a patient in a fed state, the capsule provides:
a mean plasma tretinoin Cₘₐₓ of at least about 150 ng/mL, or at least about 160 ng/mL, or at least about 180 ng/mL, or at least about 185 ng/mL, or at least about 190 ng/mL, or at least about 200 ng/mL, or at least about 210 ng/mL, or at least about 215 ng/mL, or at least about 220 ng/mL; and/or
a mean plasma tretinoin Cₘₐₓ between about 160 and 350 ng/mL, or about 180 and 320 ng/mL, or about 180 and 300 ng/mL, or about 180 and 290 ng/mL, or about 185 and 289 ng/mL, or about 190 and 290 ng/mL, or about 190 and 280 ng/mL, or about 190 and 270 ng/mL, or about 200 and 270 ng/mL, or about 200 and 260 ng/mL, or about 200 and 250 ng/mL, or about 200 and 240 ng/mL, or about 210 and 240 ng/mL, or about 220 and 240 ng/mL.

18. The pharmaceutical composition of claim 1 wherein the capsule is a 12 mg active agent capsule or one or more capsules providing a dose equivalent to a 12 mg active agent capsule and when the capsule is administered to a patient in a fed state, the capsule provides:
a mean plasma tretinoin AUC_{0-∞} baseline corrected of at least about 300 ng·h/mL, or at least about 320 ng·h/mL, or at least about 330 ng·h/mL, or at least about 340 ng·h/mL, or at least about 350 ng·h/mL, or at least about 360 ng·h/mL, or at least about 370 ng·h/mL, or at least about 380 ng·h/mL, or at least about 390 ng·h/mL, or at least about 400 ng·h/mL; and/or
a mean plasma tretinoin AUC_{0-∞} baseline corrected of between about 290 and 562 ng·h/mL, or between about 300 and 550 ng·h/mL, or between about 310 and 530 ng·h/mL, or between about 320 and 520 ng·h/mL, or between about 320 and 510 ng·h/mL, or between about 324 and 506 ng·h/mL.

19. The pharmaceutical composition of claim 1 wherein the capsule is a 12 mg active agent capsule, or one or more capsules providing a dose equivalent to a 12 mg active agent capsule, and when the capsule is administered to a patient in a fasted state the capsule provides:
a mean plasma tretinoin Cₘₐₓ of at least about 40 ng/mL, or at least about 50 ng/mL, or at least about 60 ng/mL, or at least about 70 ng/mL, or at least about 80 ng/mL, or at least about 90 ng/mL, or at least about 100 ng/mL, or at least about 110 ng/mL, or at least about 120 ng/mL, or at least about 130 ng/mL, or at least about 135 ng/mL, or at least about 140 ng/mL; and/or
a mean plasma tretinoin Cₘₐₓ between about 60 and 250 ng/mL, or between about 62 and 244 ng/mL, or between about 70 and 240 ng/mL, or between about 80 and 230 ng/mL, or between about 90 and 220 ng/mL, or between about 100 and 210 ng/mL, or between about 100 and 200 ng/mL, or between about 110 and 190 ng/mL, or between about 115 and 180 ng/mL, or between about 120 and 170 ng/mL, or between about 130 and 160 ng/mL, or between about 140 and 150 ng/mL.

20. The pharmaceutical composition of claim 1 wherein the capsule is a 12 mg active agent capsule, or one or more capsules providing a dose equivalent to a 12 mg active agent capsule, and when the capsule is administered to a patient in a fasted state the capsule provides:
a mean plasma tretinoin AUC_{0-∞} baseline corrected of at least about 100 ng·h/mL, or at least about 120 ng·h/mL, or at least about 140 ng·h/mL, or at least about 160 ng·h/mL, or at least about 180 ng·h/mL, or at least about 200 ng·h/mL, or at least about 220 ng·h/mL, or at least about 240 ng·h/mL, or at least about 258 ng·h/mL, or at least about 260 ng·h/mL, or at least about 280 ng·h/mL, or at least about 300 ng·h/mL, or at least about 310 ng·h/mL, or at least about 315 ng·h/mL, or at least about 320 ng·h/mL; and/or
a mean plasma tretinoin AUC_{0-∞} baseline corrected of between about 100 and 500 ng·h/mL, or between about 150 and 450 ng·h/mL, or between about 200 and 420 ng·h/mL, or between about 240 and 420 ng·h/mL, or between about 258 and 404 ng·h/mL, or between about 260 and 390 ng·h/mL, or between about 280 and 360 ng·h/mL.

21. (canceled)

22. The pharmaceutical composition of claim 1 wherein the capsule provides release of about 30 to 80%, or about 35 to 60%, or about 38 to 60% of the active in vitro at about 10 minutes; and/or
the capsule provides release of about 60 to 100%, or about 65 to 100%, or about 66 to 100%, of the active in vitro at about 15 minutes; and/or
the capsule provides release of about 70 to 100%, or about 75 to 100%, or about 76 to 100%, of the active in vitro at about 20 minutes; and/or
the capsule provides release of about 70 to 100%, or about 75 to 100%, or about 79 to 100%, of the active in vitro at about 30 minutes; and/or
the capsule provides release of about 75 to 100%, or about 80 to 100%, of the active in vitro at about 45 minutes.

23. (canceled)

24. (canceled)

25. A method of making a capsule fill for a pharmaceutical composition in capsule form, the method comprising the steps:
adding an active agent selected from the group consisting of tretinoin, pharmaceutically acceptable salts thereof, and combinations thereof to at least one carrier and at least one viscosity modifier,
wherein the active agent has a particle size distribution of D[v,0.5] between about 1 and 10 μm, and
wherein the at least one viscosity modifier is present in the capsule fill in a range of about 10 to 25% w/w.

26. (canceled)

27. A pharmaceutical composition in a capsule form comprising a capsule fill, wherein the capsule fill comprises:
a therapeutically effective amount of an active agent selected from the group consisting of tretinoin, pharmaceutically acceptable salts thereof, and combinations thereof;
at least one carrier;
optionally at least one viscosity modifier;
optionally at least one chelating agent; and
optionally at least one antioxidant;
wherein the capsule comprises 12 mg of tretinoin and when the capsule is administered to a patient in a fasted state the capsule provides at least one pharmacokinetic parameter selected from the group consisting of:
a mean plasma tretinoin Cₘₐₓ baseline corrected of between about 62 and 243 ng/mL; and
a mean plasma tretinoin AUC_{0-∞} baseline corrected of between about 165 and 507 ng·h/mL.

28. (canceled)
